(19) United States
(12) Patent Application Publication
(10) Pub. No.: US 2026/0232713 A1
(43) Pub. Date: Aug. 13, 2026

(54) PLECOMACROLIDE DERIVATIVES FOR MHC-I NEF DOWNMODULATION

(71) Applicant: David H. Sherman, Ann Arbor, MI (US)

(72) Inventor: David H. Sherman, Ann Arbor, MI (US)

(21) Appl. No.: 19/094,350

(22) PCT Filed: Oct. 2, 2023
(86) PCT No.: PCT/US2023/034275

§ 371 Date: Mar. 28, 2025

Related U.S. Application Data
(60) Provisional application No. 63/412,205, filed on Sep. 30, 2022.

(51) Int. Cl. A61K 31/7048; A61K 31/365; A61P 31/18; C07D 407/06; C07D 407/14; C07H 17/04

(52) U.S. Cl. CPC A61K 31/7048; A61K 31/365; A61P 31/18; C07D 407/06; C07D 407/14; C07H 17/04

(57) ABSTRACT

This disclosure relates generally to inhibitors of MHC-I downmodulation, and methods of treating or preventing an HIV infection by administering the inhibitors to a patient in need of treatment thereof.

### BACKGROUND

### Field of the Disclosure

This disclosure relates generally to inhibitors of MHC-I downmodulation, and methods of treating or preventing an HIV infection by administering the inhibitors to a patient in need of treatment thereof.

### BRIEF DESCRIPTION OF RELATED TECHNOLOGY

The development of combination antiretroviral therapy (cART) has improved the outcome for individuals with human immunodeficiency virus (HIV). However, HIV is able to persist even during cART by establishing a latent reservoir in a subset of infected cells (Siliciano et al., 2003). To fully eliminate HIV infected cells and cure the infection, a two-pronged approach is necessary to first reactivate this viral reservoir and then stimulate CTL-mediated clearance of virally infected cells. One challenge to this approach is that HIV infected cells evade clearance by anti-HIV cytotoxic T-lymphocytes (CTLs) due in part to the activity of the HIV accessory protein Nef (Collins et al., 1998). CTL recognition and killing of infected cells depends on expression of host MHC-I proteins on the cell surface. Following HIV infection, the HIV-Nef protein downmodulates MHC-I HLA-A and —B, allowing the virus to evade recognition (Collins et al., 1998). To downmodulate MHC-1, Nef binds to the cytoplasmic tail of MHC-1 and redirects it to the lysosome for degradation thereby blocking the transport of antigens to the cell membrane (Robert-Guroff et al., 1990; Roeth et al., 2004). Concanamycin A (CMA, 1) reverses this activity of Nef, normalizing MHC-I expression and allowing anti-HIV CTLs to effectively eliminate HIV infected cells in vitro (Painter et al., 2020). Thus far, it has been found that CMA acts by reducing the interaction between Nef, MHC-1, and clathrin adaptor protein 1 (AP-1) that is necessary to alter MHC-I trafficking in HIV infected cells (Painter et al., 2020). However, the precise molecular mechanism by which CMA reduces Nef-MHC-I-AP-1 complex formation and whether V-ATPase plays a role in this bioactivity remains unknown.

While CMA inhibits Nef activity at sub-nanomolar (nM) concentrations, its utility as a potential anti-HIV therapeutic requires identifying a derivative with lower toxicity. Because CMA has a much lower IC50 for Nef inhibition than lysosomal neutralization, we hypothesized the existence of separable CMA inhibitory targets. If this hypothesis is true, it should be possible to reduce toxicity by generating derivatives with maximal anti-Nef potency and minimal lysosomal neutralization. Therefore, a need remains for chemically related molecules with Nef inhibitory activity, such as derivatives of CMA, having lower toxicity.

### SUMMARY

The present disclosure generally relates to methods of treating HIV, to methods of inhibiting the replication of HIV viruses, to methods of reducing the amount of HIV viruses in a patient, and to compounds and compositions that can be employed for such methods.

In one aspect, the disclosure provides compounds of Formula (I) and pharmaceutically acceptable salts thereof:

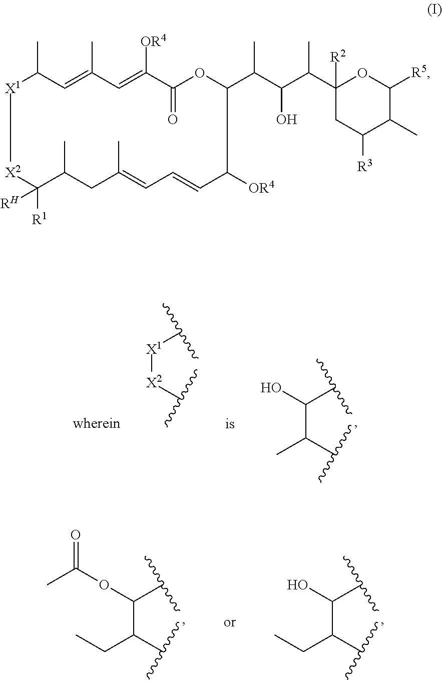

or represents a direct bond; R^{H} is H; R¹ is OH, C(O)C₁₋₃ alkyl, or OC(O)C₁₋₃ alkyl, or R^{H} and R¹ together with the carbon to which they are attached form an oxo (═O) group; R² is H, OH, or OCH₃; R³ is CHO, C(O)C₁₋₃ alkyl, OC(O)R⁶ or a modified sugar; each R⁴ is independently H or C₁₋₆alkyl; R⁵ is C₁₋₆alkyl or C₂₋₆alkenyl; and R⁶ is H, C₁₋₆haloalkyl, or C₂₋₆ alkynyl. In some instances,

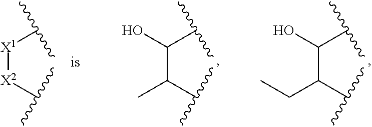

or represents a direct bond; R^{H} is H; R¹ is OH or OC(O)C₁₋₃ alkyl, or R^{H} and R¹ together with the carbon to which they are attached form an oxo (═O) group; R² is H, OH, or OCH₃; R³ is OC(O)R⁶ or a modified sugar; each R⁴ is independently H or C₁₋₆alkyl; R⁵ is C₁₋₆alkyl or C₂₋₆alkenyl; and R⁶ is H, C₁₋₆haloalkyl, or C₂₋₆ alkynyl.

In some cases, the compounds are compounds of Formula (Ia), (Ib), or (Ic):

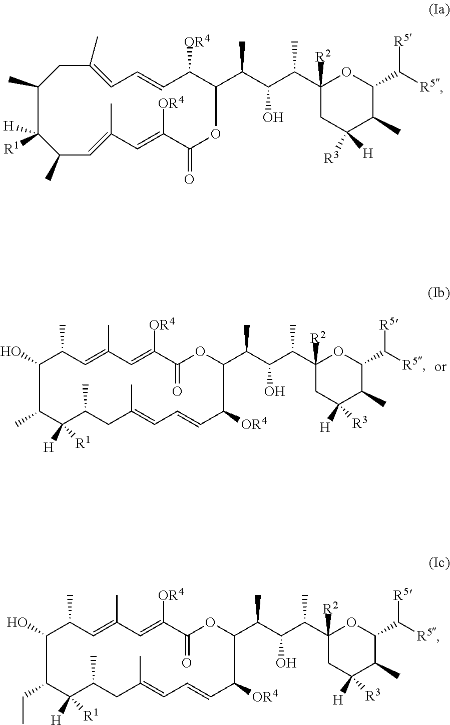

wherein (i) R^{5′} is C₂₋₅alkenyl and R^{5″} is H, or (ii) both R^{5′} and R^{5″} are C₁₋₂alkyl.

Further provided are methods of administering to a patient a safe and effective amount of a compound disclosed herein, e.g., as represented by Formulas (I), (Ia), (Ib), (Ib), or a compound of Table A, and pharmaceutically acceptable salts thereof.

Also provided are methods of modulating HIV Nef in a subject in need thereof by contacting said HIV Nef with a safe and effective amount of a compound as disclosed herein, e.g., as represented by Formulas (I), (Ia), (Ib), (Ib), or a compound of Table A, and pharmaceutically acceptable salts thereof. In some cases, modulating HIV Nef includes administering to a patient a safe and effective amount of a compound as disclosed herein e.g., as represented by Formulas (I), (Ia), (Ib), (Ib), or a compound of Table A, and pharmaceutically acceptable salts thereof.

Further provided are methods of treating an HIV Nef-associated disorder in a host by administering a safe and effective amount of a compound as disclosed herein, e.g., as represented by Formulas (I), (Ia), (Ib), (Ib), or a compound of Table A, and pharmaceutically acceptable salts thereof.

Further provided are methods of treating HIV infection in a patient, comprising administering to said patient a safe and effective amount of a compound as disclosed herein, e.g., as represented by Formulas (I), (Ia), (Ib), (Ib), or a compound of Table A, and pharmaceutically acceptable salts thereof.

Further provided are methods of reducing an HIV reservoir in a patient, comprising administering to said patient a safe and effective amount of a compound as disclosed herein, e.g., as represented by Formulas (I), (Ia), (Ib), (Ib), or a compound of Table A, and pharmaceutically acceptable salts thereof. Also provided are methods of eliminating an HIV reservoir in a patient, comprising administering to said patient a safe and effective amount of a compound as disclosed herein, e.g., as represented by Formulas (I), (Ia), (Ib), (Ib), or a compound of Table A, and pharmaceutically acceptable salts thereof.

Also provided are pharmaceutical compositions comprising a compound as disclosed herein, e.g., as represented by any of Formulas (I), (Ia), (Ib), (Ib), or a compound of Table A, and pharmaceutically acceptable salts thereof, or a pharmaceutically acceptable salt thereof, and a pharmaceutically acceptable excipient, carrier, adjuvant or vehicle.

Further provided herein are uses of a compound described herein for the manufacture of a medicament for treating HIV infection in a patient, for reducing an HIV reservoir in a patient, or for eliminating an HIV reservoir in a patient.

### DETAILED DESCRIPTION

Provided herein are analogs of plecomacrolides, such as analogs of Bafilomycins (Bafs) and Concanamycins. For the current structure-activity relationship analysis, a panel of macrolactones, macrolides, and plecomacrolides was screened against Nef expressing primary T cells to perform an in-depth analysis of functional group patterns needed for separating Nef and V-ATPase inhibitory activities (expanding neutralization window). In embodiments, the analogs of plecomacrolides disclosed herein comprise modified 18-membered ring plecomacrolides that retain single-digit nM potency and provide substantive reduction in V-ATPase inhibition, pointing the way toward final lead optimization.

Certain compounds are referred to herein by numbers, which are explained below.

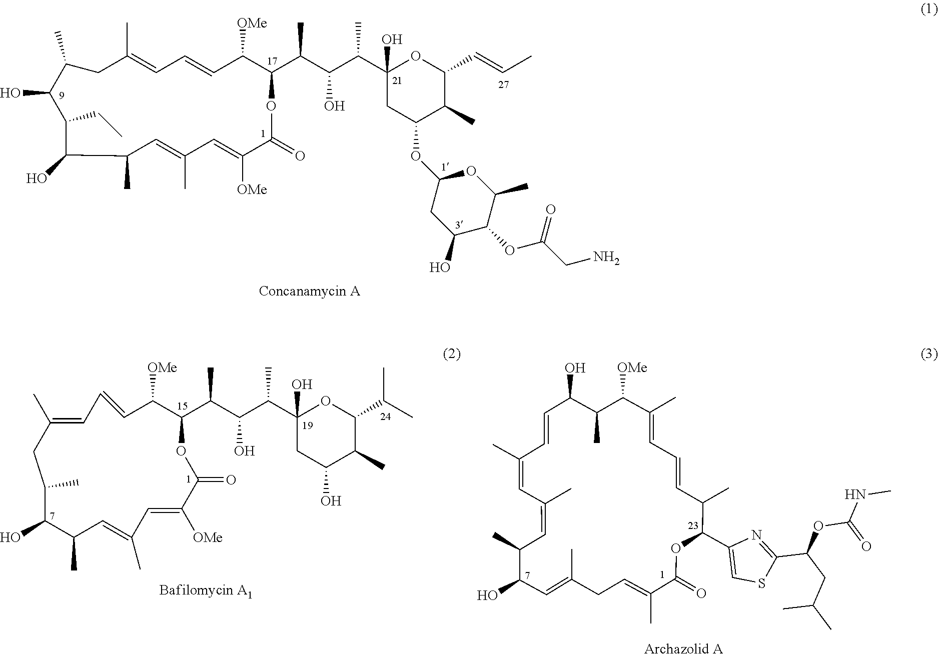

TABLE 2

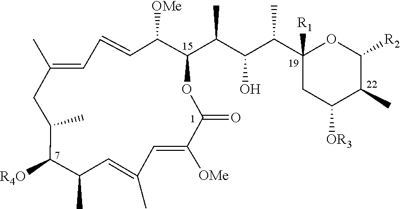

(2, S4-6, S9-15, S17-19, S27-29, S59-61)
(S8)

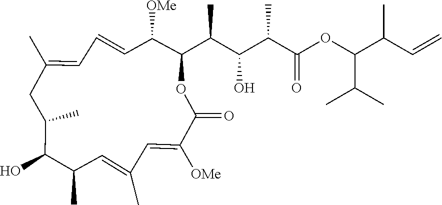 [SMILES: C=CC(C)C(OC(=O)[C@@H](C)[C@H](O)[C@H](C)[C@H]1OC(=O)/C(OC)=C/C(C)=C/[C@@H](C)[C@@H](O)[C@@H](C)C/C(C)=C/C=C/[C@@H]1OC)C(C)C]

| Name | | | | |
|---|---|---|---|---|
| Bafilomycin A₁ | 2 | 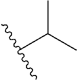 [SMILES: *C(C)C] | OH | H |
| Ring Open Bafilomycin A₁ | S8 | — | — | — |
| 21-acetyl Bafilomycin A₁ | S9 | 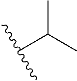 [SMILES: *C(C)C] | 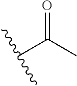 [SMILES: *C(C)=O] | H |
| 21-pentanoate Bafilomycin A₁ | S10 | 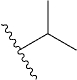 [SMILES: *C(C)C] | 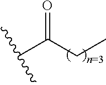 | H |
| 21-nonanoate Bafilomycin A1 | S11 | 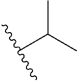 [SMILES: *C(C)C] | 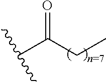 | H |
| 21-penta-2,4-dienoate Bafilomycin A₁ | S12 | 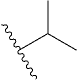 [SMILES: *C(C)C] | 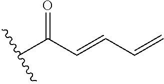 [SMILES: *C(=O)/C=C/C=C] | H |
| 21-benzoate Bafilomycin A₁ | S13 | 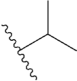 [SMILES: *C(C)C] | 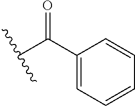 [SMILES: *C(=O)c1ccccc1] | H |
| Leucanicidin | S14 | 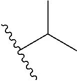 [SMILES: *C(C)C] | 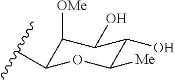 [SMILES: *C1OC(C)C(O)C(O)C1OC] | H |
| PC-766B | S15 | 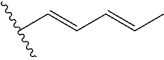 [SMILES: */C=C/C=C/C] | 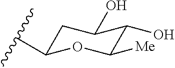 [SMILES: *C1CC(O)C(O)C(C)O1] | H |
| Elaiophylin | S16 | — | — | — |
| 7-one Bafilomycin A₁ | S17 | 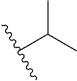 [SMILES: *C(C)C] | OH | O |
| 7,21-one Bafilomycin A₁ | S18 | 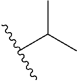 [SMILES: *C(C)C] | O | OT |

TABLE 3

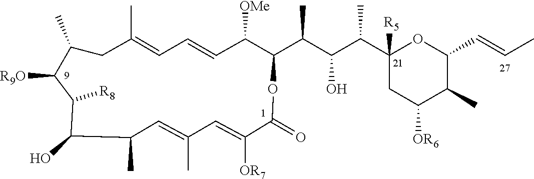

(1, 15-25)

| Name | # | R₅ | R₆ | R₇ | R₈ | R₉ |
|---|---|---|---|---|---|---|
| CMA | 1 | OH | 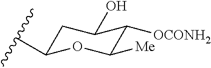 | Me | Et | H |
| CMB | 4 | OH | 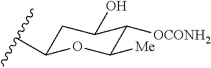 | Me | Me | H |
| CMC | 5 | OH | 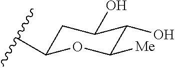 [SMILES: *C1CC(O)C(O)C(C)O1] | Me | Et | H |
| CMF | 6 | OH | H | Me | Et | H |
| CMX | 7 | OH | H | Me | Me | H |
| 21-Deoxy-16- | S25 | H | H | H | Et | H |
| Hydroxy CMF | | | | | | |
| 3′-Pentanoate CMA | 8 | OH | 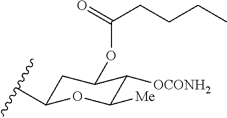 | Me | Et | H |
| 3′-Pentanoate CMB | 9 | OH | 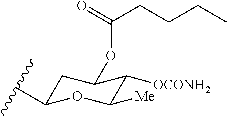 | Me | Me | H |
| 3′,9- dipentanoate CMB | 10 | OH | 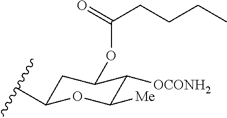 | Me | Me | 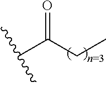 |
| 3′-nonanoate CMB | 11 | OH | 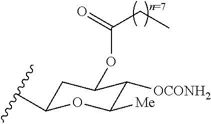 | Me | Me | H |
| 3′-Acetyl CMB | 12 | OH | 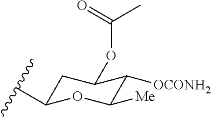 | Me | Me | H |
| 3′,9-diacetyl CMB | 13 | OH | 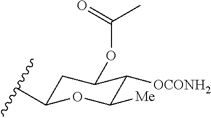 | Me | Me | 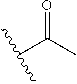 [SMILES: *C(C)=O] |
| 3′-Acetyl CMA | 14 | OH | 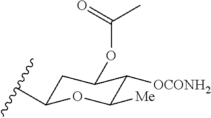 | Me | Et | H |
| 3′,9-diacetyl CMA | 15 | OH | 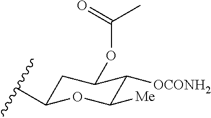 | Me | Et | 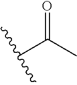 [SMILES: *C(C)=O] |

TABLE S1
(S29-30)

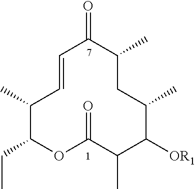

(S3-4)

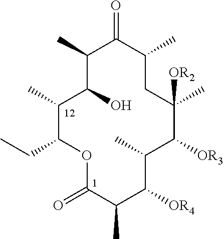

(S5)

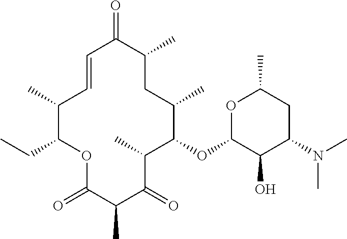 [SMILES: CC[C@H]1OC(=O)[C@H](C)C(=O)[C@H](C)[C@@H](O[C@@H]2O[C@H](C)C[C@H](N(C)C)[C@H]2O)[C@@H](C)C[C@@H](C)C(=O)/C=C/[C@H]1C]

(S6)

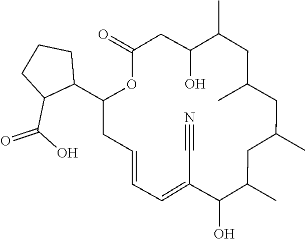 [SMILES: CC1CC(C)CC(C)C(O)/C(C#N)=C/C=C/CC(C2CCCC2C(=O)O)OC(=O)CC(O)C(C)C1]

(S7)

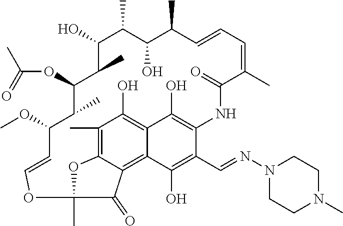

(S8)

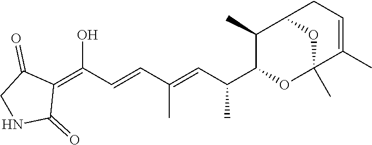 [SMILES: CC1=CC[C@H]2O[C@]1(C)O[C@H]([C@H](C)/C=C(C)/C=C/C(O)=C1/C(=O)CNC1=O)[C@H]2C]

(S9)

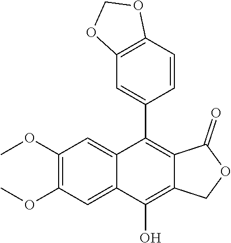 [SMILES: COc1cc2c(O)c3c(c(-c4ccc5c(c4)OCO5)c2cc1OC)C(=O)OC3]

| Compound | # | R₁ | R₂ | R₃ | R₄ |
|---|---|---|---|---|---|
| 10-deoxymethynolide | S29 | H | — | — | — |
| YC-17 | S30 | 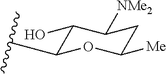 [SMILES: *C1OC(C)CC(N(C)C)C1O] | — | — | — |
| 6-deoxyerythronolide B | S31 | — | OH | H | H |
| Erythromycin B | S32 | — | OH | 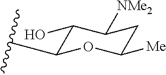 [SMILES: *C1OC(C)CC(N(C)C)C1O] | 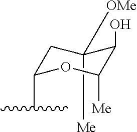 [SMILES: *C1CC(C)(OC)C(O)C(C)O1] |
| Narbomycin | S33 | — | — | — | — |
| Borrelidin | S2 | — | — | — | — |
| Rifampicin | S26 | — | — | — | — |
| Tirandamycin C | S8 | — | — | — | — |
| Diphyllin | S1 | — | — | — | — |

TABLE S4

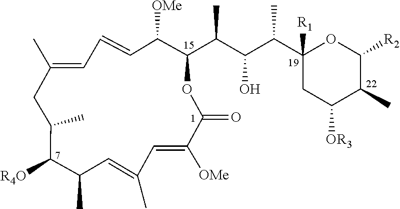

(2, S4-6, S9-15, S17-19, S27-29, S59-61)
(S8)

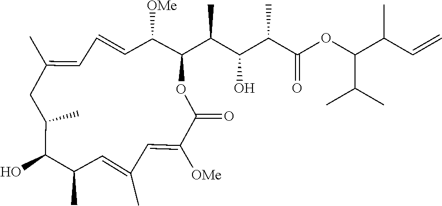 [SMILES: C=CC(C)C(OC(=O)[C@@H](C)[C@H](O)[C@H](C)[C@H]1OC(=O)/C(OC)=C/C(C)=C/[C@@H](C)[C@@H](O)[C@@H](C)C/C(C)=C/C=C/[C@@H]1OC)C(C)C]

(12)

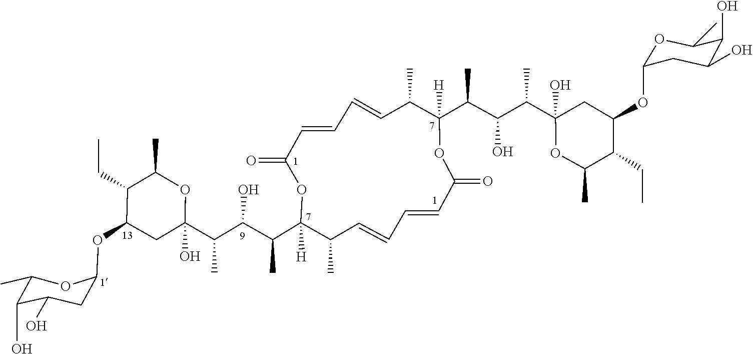 [SMILES: CC[C@@H]1[C@@H](C)O[C@@](O)([C@@H](C)[C@H](O)[C@H](C)[C@H]2OC(=O)/C=C/C=C/[C@H](C)[C@@H]([C@@H](C)[C@@H](O)[C@H](C)[C@@]3(O)C[C@@H](OC4CC(O)C(O)C(C)O4)[C@H](CC)[C@@H](C)O3)OC(=O)/C=C/C=C/[C@@H]2C)C[C@H]1OC1CC(O)C(O)C(C)O1]

| Name | # | R₁ | R₂ | R₃ | R₄ |
|---|---|---|---|---|---|
| Bafilomycin A₁ | 2 | OH | 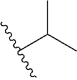 [SMILES: *C(C)C] | OH | H |
| Bafilomycin C | S59 | OH | 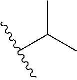 [SMILES: *C(C)C] | 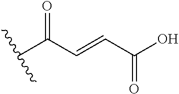 [SMILES: *C(=O)/C=C/C(=O)O] | H |
| Bafilomycin B | S60 | OH | 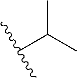 [SMILES: *C(C)C] | 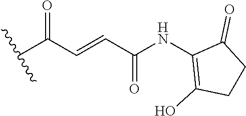 [SMILES: *C(=O)/C=C/C(=O)NC1=C(O)CCC1=O] | H |
| Ring Open | S8 | — | — | — | — |
| Bafilomycin A1 | | | | | |
| 21-Formyl Baf | A1 S61 | OH | 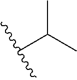 [SMILES: *C(C)C] | 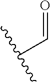 [SMILES: *C=O] | H |
| 21-acetyl Bafilomycin A₁ | S9 | OH | 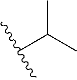 [SMILES: *C(C)C] | 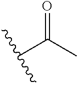 [SMILES: *C(C)=O] | H |
| 21-pentanoate Bafilomycin A₁ | S10 | OH | 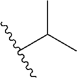 [SMILES: *C(C)C] | 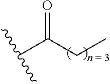 | H |
| 21-nonanoate Bafilomycin A₁ | S11 | OH | 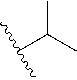 [SMILES: *C(C)C] | 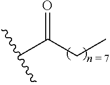 | H |
| 21-penta-2,4- dienoate Bafilomycin A₁ | S14 | OH | 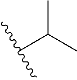 [SMILES: *C(C)C] | 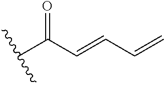 [SMILES: *C(=O)/C=C/C=C] | H |
| 21-Penta-3- enoate Baf A₁ | S29 | OH | 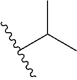 [SMILES: *C(C)C] | 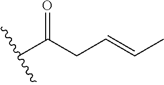 [SMILES: *C(=O)C/C=C/C] | H |
| 21-benzoate Bafilomycin A₁ | S13 | OH | 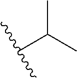 [SMILES: *C(C)C] | 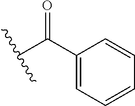 [SMILES: *C(=O)c1ccccc1] | H |
| Leucanicidin | S14 | OH | 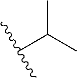 [SMILES: *C(C)C] | 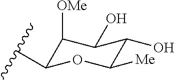 [SMILES: *C1OC(C)C(O)C(O)C1OC] | H |
| PC-766B | S15 | OH | 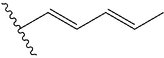 [SMILES: */C=C/C=C/C] | 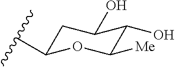 [SMILES: *C1CC(O)C(O)C(C)O1] | H |
| Elaiophylin | S16 | — | — | — | — |
| 7-one Bafilomycin A₁ | S17 | OH | 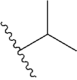 [SMILES: *C(C)C] | OH | O |
| 7,21-one Bafilomycin A₁ | S18 | OH | 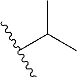 [SMILES: *C(C)C] | O | O |
| Bafilomycin A₂ | S4 | OMe | 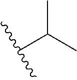 [SMILES: *C(C)C] | OH | H |
| 19-Deoxy Bafilomycin A₁ | S5 | H | 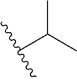 [SMILES: *C(C)C] | OH | H |
| 19-Deoxy-20-ene | S7 | H | — | — | — |
| Bafilomycin A₁ | | | | | |
| 19-Deoxy-21- Methoxy B Bafilomycin A₁ | S6 | H | 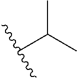 [SMILES: *C(C)C] | Me | H |
| 19-Deoxy-21- allylester Bafilomycin A₁ | S27 | H | 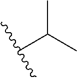 [SMILES: *C(C)C] | 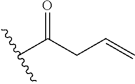 [SMILES: *C(=O)CC=C] | H |
| 19-Deoxy-21(4- Pentynoate) Bafilomycin A₁ | S65 | H | 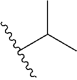 [SMILES: *C(C)C] | 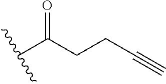 [SMILES: *C(=O)CCC#C] | H |
| 7,21-Diacetyl Bafilomycin A1 | S19 | H | 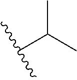 [SMILES: *C(C)C] | 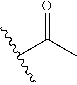 [SMILES: *C(C)=O] | 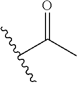 [SMILES: *C(C)=O] |
| 3′,4-diacetyl CMC | S48 | OH | 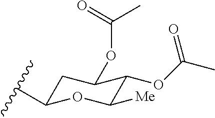 [SMILES: *C1CC(OC(C)=O)C(OC(C)=O)C(C)O1] | Me | Et |
| 3′,4′,9-triaceyl CMC | S49 | OH | 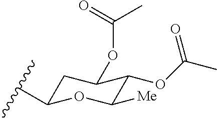 [SMILES: *C1CC(OC(C)=O)C(OC(C)=O)C(C)O1] | Me | Et |
| 23-Acetyl CMF | S50 | OH | 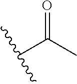 [SMILES: *C(C)=O] | Me | Et |
| 9,23-diacetyl CMF | S51 | OH | 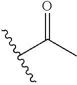 [SMILES: *C(C)=O] | Me | Et |
| 21-Formyl Baf A₁ | S61 | OH | 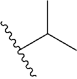 [SMILES: *C(C)C] | 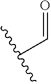 [SMILES: *C=O] | H |
| 3′-one CMA | S63 | OH | 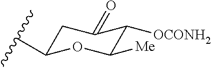 | Me | Et |
| Virustomycin | S64 | OH | 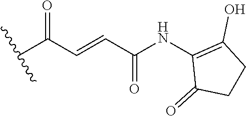 [SMILES: *C(=O)/C=C/C(=O)NC1=C(O)CCC1=O] | Me | Et |

### Screening for Nef and V-ATPase Inhibition Reveals the Importance of Macrolactone Ring Size

To investigate structure activity relationships (SAR) for Nef inhibitory activity across a variety of related macrocyclic compounds, a panel of 74 molecules with varying ring sizes and additional select structural modifications was tested, along with two known V-ATPase inhibitors, diphyllin and archazolid A, that are structurally unrelated to plecomacrolides but share this “off target” (V-ATPase) inhibitory activity (Sørensen et al., 2007). Inhibition of HIV-Nef was measured by assessing its capacity to downmodulate MHC-I HLA-A2 on the surface of primary CD4+ T cells 24-hours after treatment with each compound. V-ATPase inhibition was assessed at the same time point using lysosomal acidification as an indicator of V-ATPase activity. This was measured using a flow cytometric assay that quantified Lysotracker mean fluorescence intensity as previously described (Painter et al., 2020). Cell viability was measured at 24-hour and 72-hour time points using a previously described flow cytometric assay (Painter et al., 2020).

All compounds tested with relatively small macrocyclic rings (12- and 14-membered macrolactones) failed to show potent Nef or V-ATPase inhibitory activity at concentrations below 1000 nM, which was the upper limit of consideration in this SAR study. In addition, none of the non-macrolide compounds tested, including the V-ATPase inhibitor diphyllin, had a Nef IC₅₀ below 1000 nM. A small number of compounds, such as borrelidin, were toxic at 24-hours making any determination of Nef inhibitory activity unreliable. Of the two 24-membered macrolactones that were tested, only archazolid A (3), an inhibitor that binds to the same subunit of V-ATPase as the plecomacrolides, had a Nef IC₅₀ less than 1000 nM (Table B) (Keon et al., 2022). Indeed, 3 was more potent than 2, achieving the sub nM potency observed with 1 (Table B). In comparison to 1, compound 3 had a smaller therapeutic window separating Nef IC₅₀ and TC₅₀ (6.6 & 1.4-fold, respectively) as well as a smaller neutralization window between Nef and V-ATPase IC₅₀s (9.4 & 2.9-fold, respectively; Table B). Nevertheless, 3 is notable in that this 24-membered macrocycle was the only structurally distinct compound from the plecomacrolides tested that displayed sub-nM potency (Nef IC₅₀=0.47 nM).

A detailed comparison between 1 and 2 showed that 1 displays over 60-fold more potent Nef inhibitory activity relative to 2 and has a larger therapeutic window (Table B) (Painter et al., 2020). A comparative assessment of lysosomal neutralization was conducted between these two molecules, and it was found that 1 had a greater neutralization window than 2 (9.4 & 2.9-fold respectively; Table B).

### Semi-Synthetic Modification of 16-Membered Plecomacrolides Identifies Critical Moieties Required for Nef Inhibition

### The Role of the Pyran Ring at C-19 in Bafilomycin A1

To better understand which structural variations within the plecomacrolide family members determine toxicity, Nef and V-ATPase inhibition, synthetic modifications were tested first with 2, a more readily accessible plecomacrolide, leading to a total of 15 semi-synthetic derivatives (Li et al., 2017). It has previously been reported that bafilomycin D (lacking a pyran) displayed very poor potency as a Nef inhibitor, which similarly shows low level V-ATPase inhibition (Dröse et al., 2001; Osteresch et al., 2012; Painter et al., 2020; Wang et al., 2021). An investigation of the impact of pyran ring modifications showed that C-19 methylation, deoxygenation, and/or C-21 methylation all decreased potency (increased Nef IC₅₀) with dehydration of C-20 (S40) having the greatest effect. To directly confirm the requirement of the hemiketal functionality in a semi-synthetic derivative, Mitsunobu conditions yielded “ring-opened” bafilomycin A₁ (S8; Table B) (Bowman et al., 1988). This alteration led to a 26-fold decrease in potency compared to 2 (Nef IC₅₀=310 nM; p≤0.001; Table B).

### Enhancement of Nef and V-ATPase Inhibition Through Modifications at C-21 in 16-Membered Ring Plecomacrolides

Based on observations that the pyran ring was more amenable to modifications, development efforts focused on derivatizing the C-21-OH to assess if chain extensions at this position (increasing the overall size of 2), would mimic the larger concanamycin analogs and improve potency. Interestingly, modifying C-21-OH with an acetyl group (S9) led to a 2.5-fold increase in potency compared to 2 (Nef IC₅₀=4.5 nM vs 12 nM, p≤0.05; Table B) (Bender et al., 2007). This improvement was not accompanied by a significant change in V-ATPase inhibition as the neutralization window for S9 was almost 2-fold larger than 2 (p≥0.05; Table B). Without wishing to be bound by any particular theory, introduction of esters with longer acyl chains (S10 & S11, or an unsaturated chain at C-21-OH) resulted in decreased potency (Nef IC₅₀=37 nM and 130 nM, respectively) and worsening of the neutralization window, however, incorporation of an unsaturated chain (S12) restored potency to parent compound levels (Nef IC₅₀=17 nM vs 12 nM; Table B). Again without wishing to be bound by any particular theory, incorporation of an aromatic substituent (S13) led to a decrease in potency (Nef IC₅₀=92 nM) highlighting that steric hindrance plays an important role at the C-21-OH position and compromises the biological activity of bafilomycin analogs.

In addition to the larger macrocyclic ring, 1 contains a 4β-D-rhamnose at C-23, instead of a hydroxyl at the analogous C-21 position in 2. To determine the extent to which this sugar moiety is required for Nef and V-ATPase inhibition, the activity of three 16-membered ring glycosylated plecomacrolides was examined: leucanicidin (S14), PC-766B (S15) and elaiophylin (S16) (Table B). Interestingly, S14 and S15 were found to have single digit nM potency (Nef IC₅₀=1.2 nM and 1.2 nM, respectively), however, the increase in potency was paired with an increase in toxicity (TC₅₀=1.4 nM and 1.3 nM, respectively; Table B). Despite the presence of a (3-D-rhamnose at C-21, S16 displayed a 20-fold decrease in potency compared to 2 (Nef IC₅₀=250.0 nM vs 12 nM; p≤0.001; Table B).

### The Bafilomycin A1 C-7 Hydroxyl Group is Essential for Both Nef and V-ATPase Inhibition

A recently reported cryo-EM structure of bafilomycin A₁ (2) bound to the V₀ domain of V-ATPase highlights the importance of the hydrogen bond interaction between the C-7-OH on the macrocyclic ring and the corresponding tyrosine residue at position 144 of the c subunit (Wang et al., 2021). To investigate whether this interaction also plays a role in Nef inhibition and to confirm its requirement for V-ATPase inhibition, a Corey-Suggs oxidation was used to generate a new bafilomycin A₁ derivative bearing a keto group at C-7 as well as the corresponding C-7 and C-21 diketo derivative (S17 & S18, respectively; Table B) (Gatti et al., 1996). Without wishing to be bound by any particular theory, oxidation at C-7 was sufficient to entirely abolish both Nef and V-ATPase inhibition with IC₅₀s above the activity threshold (Table B). We also found there was a notable decrease in activities for 7,21-diacetyl bafilomycin (S19), supporting the possibility that there are steric limitations at this position.

### Moieties in 18-Membered Ring Plecomacrolides Required for Nef Inhibition

### The Role of the Concanamycin Macrolactone C-8 Position

Along with 1, there are two additional naturally occurring concanamycin analogs that are obtained concurrently by fermentation, including concanamycin B (4) that differs from 1 by a single carbon at the C-8 position and concanamycin C (5), which lacks the 4′-carbamoyl group on the sugar (Table 3; (Kinashi et al., 1984)). Without wishing to be bound by any particular theory, the ethyl vs methyl difference between 1 and 4 resulted in a 10-fold decrease in potency, however, 4 maintained sub-nM potency (Nef IC₅₀=1.2 nM vs. 0.18 nM; p≤xxx; Table B). Although both compounds had similar therapeutic windows, 4 exhibited an improved neutralization window compared to 1 (24-fold vs. 9.4-fold, respectively; Table B). By contrast, 5, which lacks the 4′-carbamoyl on the β-D-rhamnose, displayed decreased potency compared to 1 (Nef IC₅₀=0.71 nM vs. 0.18 nM; p≤0.01) and a concomitant decrease in the therapeutic and neutralization windows.

Role of the 4′-carbamoyl-β-D-rhamnose at C-23 of CMA

To further explore the role of the 4′-carbamoyl-β-D-rhamnose in 1, an aglycone derivative was synthesized and tested, concanamycin F (6) that was accessible through acid-catalyzed hydrolysis of the sugar moiety (Table B) (Bindseil and Zeeck, 1993). Without wishing to be bound by any particular theory, lack of the 4′-carbamoyl-β-D-rhamnose led to a decrease in potency compared to 1 (Nef IC₅₀=1.6 nM vs 0.18 nM; p≤0.001). This also led to 6 having a smaller neutralization window compared to 1 (4-fold vs 9.4-fold; Table B). Further highlighting the importance that the 4′-carbamoyl-β-D-rhamnose plays in Nef inhibition, the aglycone of 4, CMX (7), also displayed a reduction in potency (Nef IC₅₀=9.1 nM vs 1.2 nM; p≤0.0001; Table B). Though 6 showed a decrease in V-ATPase inhibition compared to 1 (V-ATPase IC₅₀=5.8 nM vs 1.7 nM; p≤0.001), the neutralization window for both aglycones was smaller compared to their parent compounds, since the absence of the 4′-carbamoyl-β-D-rhamnose had a greater impact on decreasing potency.

### Enhancement of Nef and V-ATPase Inhibition by Modifications at C-3′ in 18-Membered Ring Plecomacrolides

A series of acyl chains were selected to modify 1 and 4 to assess changes in inhibitory activities. Addition of longer, flexible chains to C-3′, such as a pentanoate group to each parent (8 and 9), resulted in a significant increase in the Nef IC₅₀ (0.18 to 0.75 nM and 1.2 to 3.4 nM, respectively). A similar increase in the neutralization window of 8 was also observed (9.4-fold to 15-fold).. Addition of a second pentanoate group to the C-9-OH (10) did not significantly alter potency and caused no change in the neutralization window compared to 9 (V-ATPase IC₅₀=110 nM & 130 nM respectively; Table B). However, when compared to the parent compound 4, the second addition at C-9-OH decreased V-ATPase inhibition by 5-fold (V-ATPase IC₅₀=130 nM vs 26 nM; p≤0.001; Table B). Extension of the chain in the form of a 3-nonanoate CMB derivative (23) resulted in small but statistically significant increases in Nef IC₅₀ (6.3 and 9.1 nM respectively). In contrast, a single C-3′-OH acetylation (12) improved the Nef IC₅₀ of 4 (0.66 nM vs 1.2 nM) and slightly increased the therapeutic window (8-fold vs 5-fold). Importantly, double acetylation at C-3′-OH and C-9-OH (13) maintained Nef potency (Nef IC₅₀=0.99 nM) while widening the therapeutic window (8.8-fold vs 5.5-fold), without significantly changing the neutralization window. This compound had the largest neutralization and therapeutic windows, motivating our synthesis of corresponding single and multiple acetylations of 1, 6, and 5 (yielding 14/15, S20/S21, and S22/S23, respectively). Semi-synthetic derivative 3′,9-diacetyl CMA (15) positively impacted its parent compound (1) the most, displaying a greater than two-fold improvement in the neutralization window (21-fold vs 9.4-fold, p<0.001) while maintaining a sub-single digit nanomolar Nef IC₅₀ (0.39 nM).

The 16-methoxy in 18 Membered Ring Plecomacrolides

Finally, after noting that relatively small structural variations in the macrolactone core can alter potency, analogs that had a modification on the right side of the macrocycle were tested. Without wishing to be bound by any particular theory, it has been suggested that the concanamycin C-16-OMe position is crucial for maintaining the active macrolactone conformation and retaining potent V-ATPase inhibitory properties (Dröse et al., 2001). This hypothesis was examined by converting the C-16-OMe to the C-16-OH derivative from the previously synthesized 21-deoxy concanamycin F (S24), as deoxygenation at the C-21 showed no change in potency, to form 21-deoxy-16-hydroxy concanamycin F (S25; Table B). The change at C-2 led to a complete loss of activity (over 200-fold reduction) compared to 6 (p≤0.001; Table B).

### Compounds

The present disclosure provides compounds of Formula I, and pharmaceutically acceptable salts thereof:

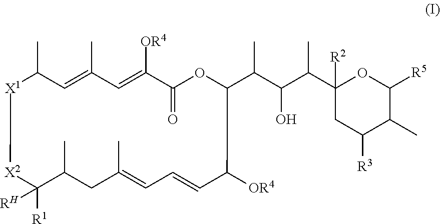

wherein

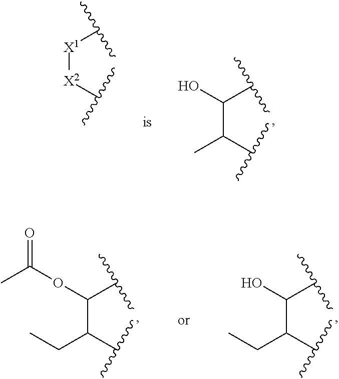

or represents a direct bond;
R^{H} is H;
R¹ is OH, C(O)C₁₋₃ alkyl, or OC(O)C₁₋₃ alkyl,
   or R^{H} and R¹ together with the carbon to which they are attached form an oxo (═O) group;
R² is H, OH, or OCH₃;
R³ is CHO, C(O)C₁₋₃ alkyl, OC(O)R⁶ or a modified sugar;
each R⁴ is independently H or C₁₋₆alkyl;
R¹ is C₁₋₆alkyl or C₂₋₆alkenyl; and
R⁶ is H, C₁₋₆haloalkyl, or C₂₋₆ alkynyl. In some instances,

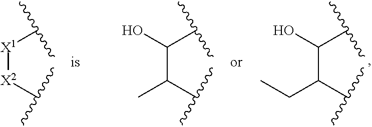

or represents a direct bond;
R^{H} is H;
R¹ is OH or OC(O)C₁₋₃ alkyl,
   or R^{H} and R¹ together with the carbon to which they are attached form an oxo (═O) group;
R² is H, OH, or OCH₃;
R³ is OC(O)R⁶ or a modified sugar;
each R⁴ is independently H or C₁₋₆alkyl;
R¹ is C₁₋₆alkyl or C₂₋₆alkenyl; and
R⁶ is H, C₁₋₆haloalkyl, or C₂₋₆ alkynyl.

In some cases,

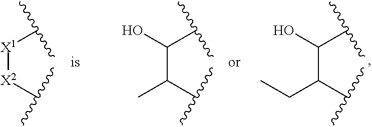

or represents a direct bond. In some cases,

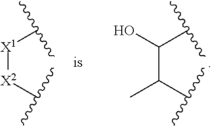

In some cases,

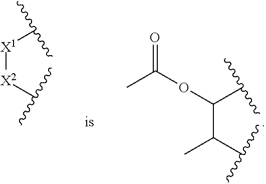

In some cases,

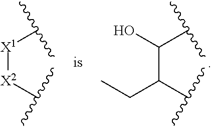

In some cases,

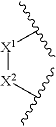

represents a direct bond. In some cases, the compound has a structure of Formula (Ia), (Ib), or (Ic):

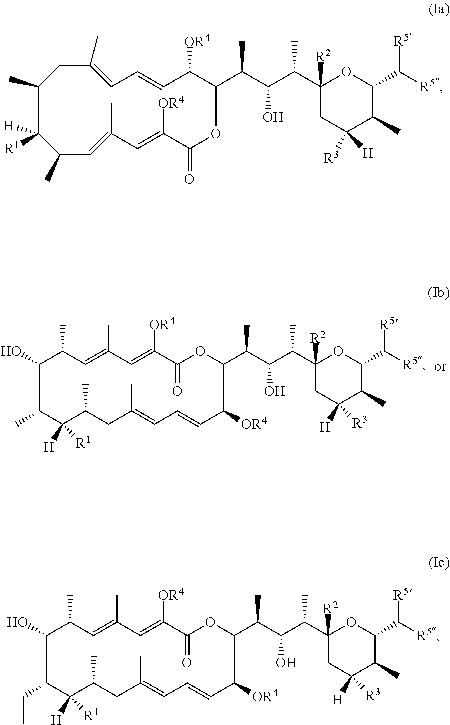

wherein (i) R^{5′} is C₂-alkenyl and R^{5″} is H, or (ii) both R^{5′} and R^{5″} are C₁₋₂alkyl. In some cases, the compound has a structure of Formula Ia

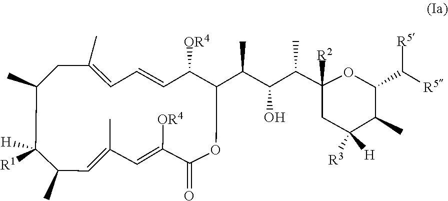

In some cases, the compound has a structure of Formula Ib:

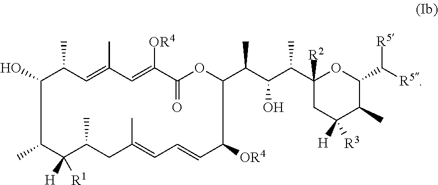

In some cases, the compound has a structure of Formula Ic:

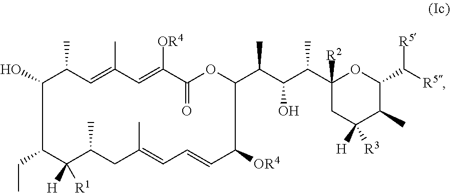

In some cases, R^{H} is H. In some cases, R¹ is C(O)C₁₋₃ alkyl. In some cases, R¹ is OH or OC(O)C₁₋₈ alkyl. In some cases, R¹ is OH. In some cases, R¹ is OC(O)C₁₋₃ alkyl. In some cases, R^{H} and R¹ together with the carbon to which they are attached form an oxo (═O) group. In some cases, R¹ is

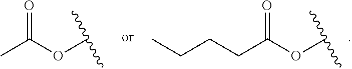

In some cases, R¹ is

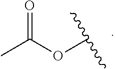 [SMILES: *OC(C)=O]

In some cases, R¹ is

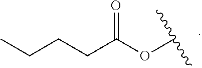 [SMILES: *OC(=O)CCCC]

In some cases, R² is H or OH. In some cases, R² is OH or OCH₃. In some cases, R² is H or OCH₃. In some cases, R² is H. In some cases, R² is OH. In some cases, R² is OCH₃.

In some cases, R³ is CHO. In some cases, R³ is C(O)C₁₋₃ alkyl. In some cases, R³ is OC(O)R⁶ or a modified sugar. In some cases, R³ is OC(O)R⁶. In some cases, R⁶ is H. In some cases, R⁶ is C₁₋₆haloalkyl. In some cases, R³ is 2-fluoroacetyl, 2-chloroacetyl, 2-bromoacetyl, or 2-bromopropanoate. In some cases, R³ is 2-fluoroacetyl. In some cases, R³ is 2-chloroacetyl. In some cases, R³ is 2-bromoacetyl. In some cases, R³ is 2-bromopropanoate. In some cases, R⁶ is C₂₋₆ alkynyl. In some cases, R³ is

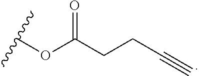 [SMILES: *OC(=O)CCC#C]

In some cases, R³ is a modified sugar. In some cases, R³ is a modified rhamnose. In some cases, R³ is a modified deoxyrhamnose. In some cases, R³ is a modified p-D-deoxyrhamnose. In some cases, R³ is

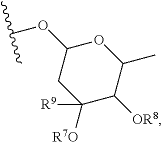

wherein R⁷ is H, C(O)C₁₋₈alkyl, C(O)C₂₋₈alkenyl, or C(O)C₂₋₈alkynyl, or OR⁷ and R⁹ together with the carbon to which they are attached form an oxo (═O) group; R³ is H, C(O)C₁₋₈alkyl, C(O)C₂₋₈alkenyl, C(O)C₂₋₈alkynyl, or C(O)NH₂; and R⁹ is H; wherein C₂₋₈alkynyl is optionally substituted with a fused 3-membered heterocycloalkyl ring comprising two nitrogen atoms. In some cases, R³ is

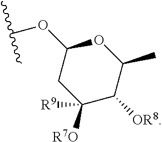

In some cases, R³ is

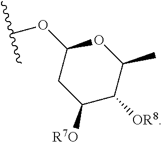

In some cases, R⁷ is H. In some cases, R⁷ is C(O)C₁₋₈alkyl, C(O)C₂₋₈alkenyl, or C(O)C₂₋₈alkynyl. In some cases, R⁷ is C(O)C₁₋₈alkyl. In some cases, R⁷ is C(O)C₂₋₈alkenyl. In some cases, R⁷ is C(O)C₂₋₈alkynyl. In some cases, R⁷ is

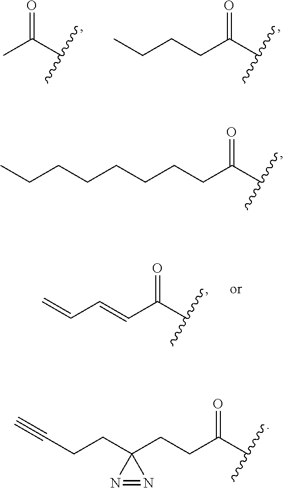

In some cases, R⁷ is

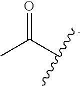 [SMILES: *C(C)=O]

In some cases, R⁷ is

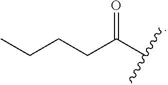 [SMILES: *C(=O)CCCC]

In some cases, R⁷ is

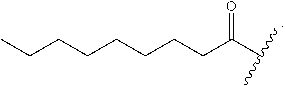 [SMILES: *C(=O)CCCCCCCC ; "."]

In some cases, R⁷ is

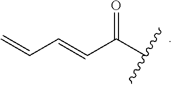 [SMILES: *C(=O)/C=C/C=C]

In some cases, R⁷ is

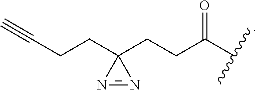 [SMILES: *C(=O)CCC1(CCC#C)N=N1]

In some cases, R⁹ is H. In some cases, OR⁷ and R⁹ together with the carbon to which they are attached form an oxo (═O) group. In some cases, R⁸ is H. In some cases, R⁸ is C(O)C₁₋₈alkyl. In some cases, R⁸ is

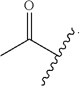 [SMILES: *C(C)=O]

In some cases, R⁸ is C(O)NH₂.

In some cases, at least one R⁴ is H. In some cases, each R⁴ is H. In some cases, at least one R⁴ is C₁₋₆alkyl. In some cases, each R⁴ is C₁₋₆alkyl. In some cases, at least one R⁴ is methyl. In some cases, each R⁴ is methyl.

In some cases, R⁵ is C₁₋₆alkyl. In some cases, R⁵ is C₃alkyl. In some cases, R⁵ is isopropyl. In some cases, R⁵ is C₂₋₆alkenyl. In some cases, R⁵ is

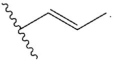 [SMILES: */C=C/C]

In some cases, R⁵ is

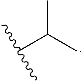 [SMILES: *C(C)C]

In some cases, R^{5′} is C₁₋₅alkyl. In some cases, In some cases, R^{5′} is methyl. In some cases, R^{5′} is C₂₋₅alkenyl. In some cases, R^{5′} is C₃alkenyl. In some cases, R^{5′} is allyl. In some cases, R^{5′} is H. In some cases, R^{5′} is C₁₋₅alkyl. In some cases, R^{5′} is methyl. In some case, R^{5′} is C₂₋₅alkenyl and R^{5′} is H. In some cases, In some cases, R^{5′} is C₂alkenyl and R^{5′} is H. In some cases, both R^{5′} and R^{5′} are C₁₋₂alkyl. In some cases, R^{5′} and R^{5′} are methyl.

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this disclosure, unless only one of the isomers is drawn specifically.

Therefore, single stereochemical isomers as well as enantiomeric, diastereomeric, cis/trans, conformational, and rotational mixtures of the present compounds are within the scope of the disclosure.

Unless otherwise indicated, all tautomeric forms of the compounds described herein are within the scope of the disclosure.

Additionally, unless otherwise indicated, structures depicted herein are also meant to include compounds that differ only in the presence of one or more isotopically enriched atoms. Discussion of an element is intended to include all isotopes of that element. For example, a substituent shown as a hydrogen includes where that hydrogen is in the deuterium or tritium isotope form, and a carbon atom can be present as a ¹³C- or ¹⁴C carbon isotope.

It is understood that selections of values of each variable are those that result in the formation of stable or chemically feasible compounds.

Also provided are compounds listed in Table A, and pharmaceutically acceptable salts thereof:

TABLE A

| Compound | |
|---|---|
| No. | Structure |
| A1 (also called S61) | 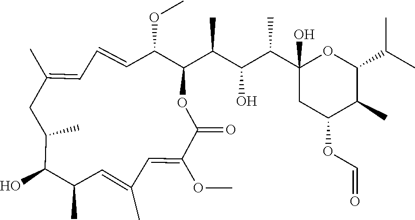 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(O)C[C@@H](OC=O)[C@H](C)[C@@H](C(C)C)O2)OC1=O] |
| A2 | 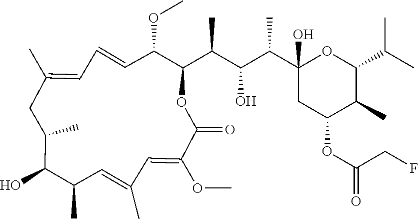 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(O)C[C@@H](OC(=O)CF)[C@H](C)[C@@H](C(C)C)O2)OC1=O] |
| A3 | 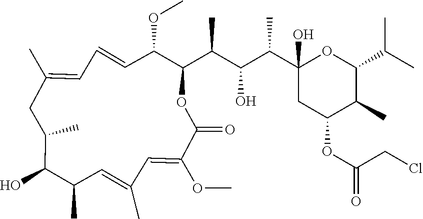 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(O)C[C@@H](OC(=O)CCl)[C@H](C)[C@@H](C(C)C)O2)OC1=O] |
| A4 | 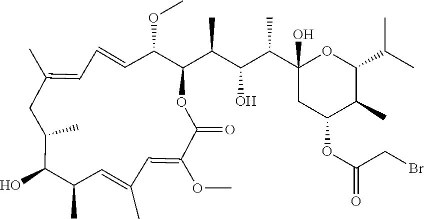 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(O)C[C@@H](OC(=O)CBr)[C@H](C)[C@@H](C(C)C)O2)OC1=O] |
| A5 | 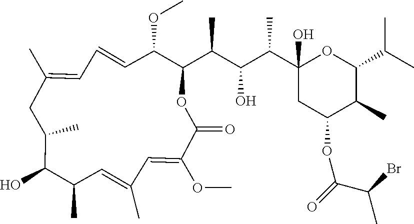 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(O)C[C@@H](OC(=O)[C@H](C)Br)[C@H](C)[C@@H](C(C)C)O2)OC1=O] |
| A6 (also called S63) | 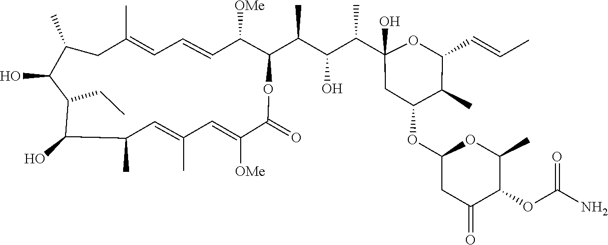 [SMILES: C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](CC)[C@@H](O)[C@H](C)C/C(C)=C/C=C/[C@@H]2OC)C[C@@H](O[C@@H]2CC(=O)[C@@H](OC(N)=O)[C@H](C)O2)[C@@H]1C] |
| A7 (also called S66) | 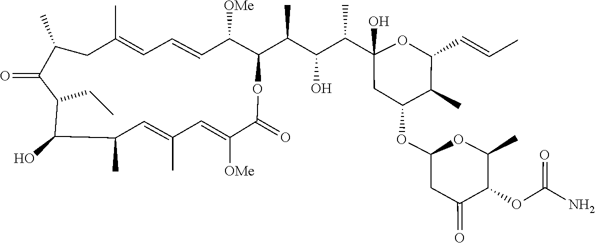 [SMILES: C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](CC)C(=O)[C@H](C)C/C(C)=C/C=C/[C@@H]2OC)C[C@@H](O[C@@H]2CC(=O)[C@@H](OC(N)=O)[C@H](C)O2)[C@@H]1C] |
| A8 (also called 12) | 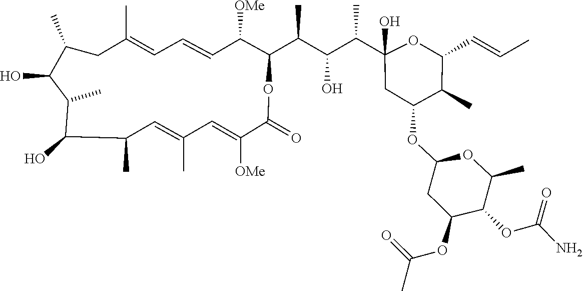 [SMILES: C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](C)[C@@H](O)[C@H](C)C/C(C)=C/C=C/[C@@H]2OC)C[C@@H](O[C@@H]2C[C@H](OC(C)=O)[C@@H](OC(N)=O)[C@H](C)O2)[C@@H]1C] |
| A9 (also called 13) | 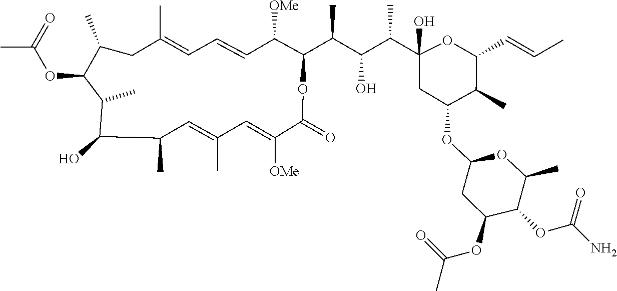 [SMILES: C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](C)[C@@H](OC(C)=O)[C@H](C)C/C(C)=C/C=C/[C@@H]2OC)C[C@@H](O[C@@H]2C[C@H](OC(C)=O)[C@@H](OC(N)=O)[C@H](C)O2)[C@@H]1C] |
| A10 (also called 9) | 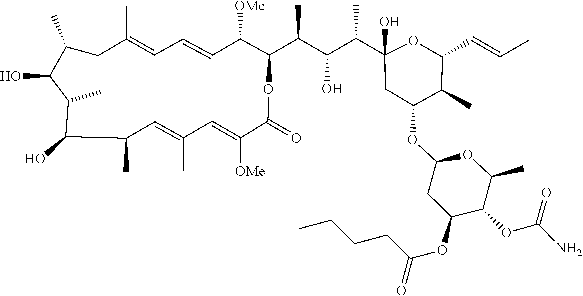 [SMILES: C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](C)[C@@H](O)[C@H](C)C/C(C)=C/C=C/[C@@H]2OC)C[C@@H](O[C@@H]2C[C@H](OC(=O)CCCC)[C@@H](OC(N)=O)[C@H](C)O2)[C@@H]1C] |
| A11 (also called 10) | 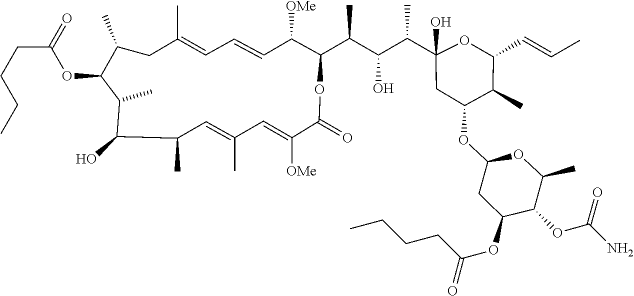 [SMILES: C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](C)[C@@H](OC(=O)CCCC)[C@H](C)C/C(C)=C/C=C/[C@@H]2OC)C[C@@H](O[C@@H]2C[C@H](OC(=O)CCCC)[C@@H](OC(N)=O)[C@H](C)O2)[C@@H]1C] |
| A12 (also called 8) | 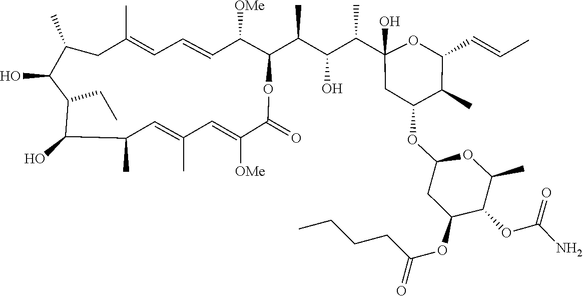 [SMILES: C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](CC)[C@@H](O)[C@H](C)C/C(C)=C/C=C/[C@@H]2OC)C[C@@H](O[C@@H]2C[C@H](OC(=O)CCCC)[C@@H](OC(N)=O)[C@H](C)O2)[C@@H]1C] |
| A13 (also called 11) | 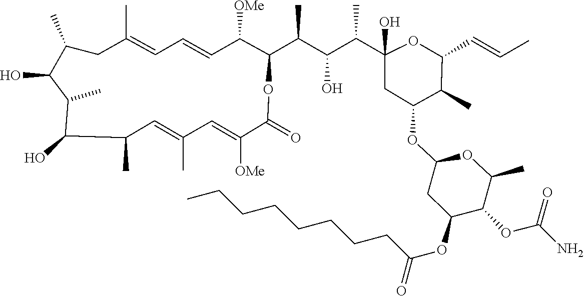 [SMILES: C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](C)[C@@H](O)[C@H](C)C/C(C)=C/C=C/[C@@H]2OC)C[C@@H](O[C@@H]2C[C@H](OC(=O)CCCCCCCC)[C@@H](OC(N)=O)[C@H](C)O2)[C@@H]1C] |
| A14 (also called 14) | 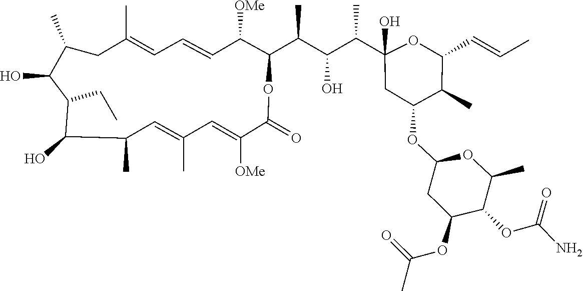 [SMILES: C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](CC)[C@@H](O)[C@H](C)C/C(C)=C/C=C/[C@@H]2OC)C[C@@H](O[C@@H]2C[C@H](OC(C)=O)[C@@H](OC(N)=O)[C@H](C)O2)[C@@H]1C] |
| A15 (also called 15) | 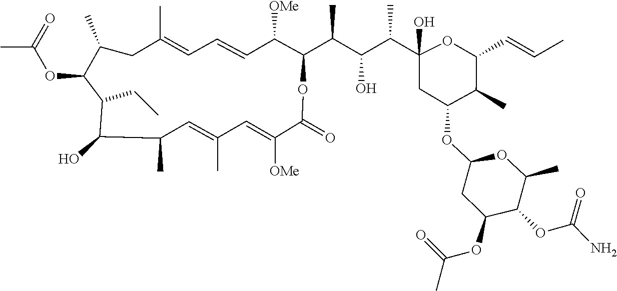 [SMILES: C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](CC)[C@@H](OC(C)=O)[C@H](C)C/C(C)=C/C=C/[C@@H]2OC)C[C@@H](O[C@@H]2C[C@H](OC(C)=O)[C@@H](OC(N)=O)[C@H](C)O2)[C@@H]1C] |
| A16 | 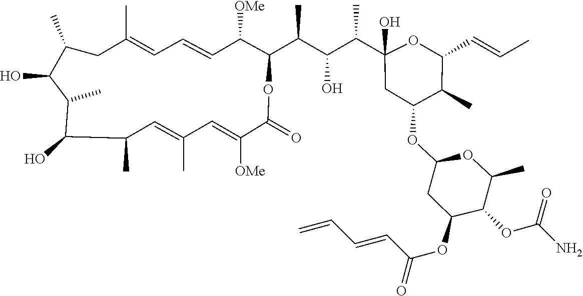 [SMILES: C=C/C=C/C(=O)O[C@H]1C[C@@H](O[C@@H]2C[C@](O)([C@@H](C)[C@H](O)[C@H](C)[C@H]3OC(=O)/C(OC)=C/C(C)=C/[C@@H](C)[C@@H](O)[C@@H](C)[C@@H](O)[C@H](C)C/C(C)=C/C=C/[C@@H]3OC)O[C@H](/C=C/C)[C@H]2C)O[C@@H](C)[C@@H]1OC(N)=O] |
| A17 (also called S48) | 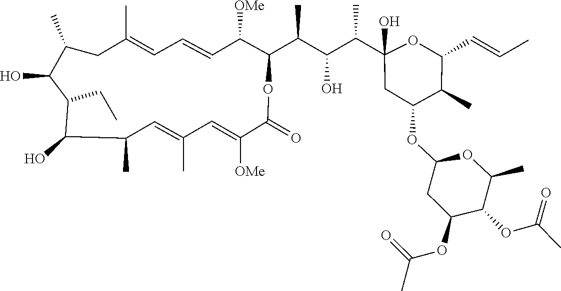 [SMILES: C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](CC)[C@@H](O)[C@H](C)C/C(C)=C/C=C/[C@@H]2OC)C[C@@H](O[C@@H]2C[C@H](OC(C)=O)[C@@H](OC(C)=O)[C@H](C)O2)[C@@H]1C] |
| A18 (also called S49) | 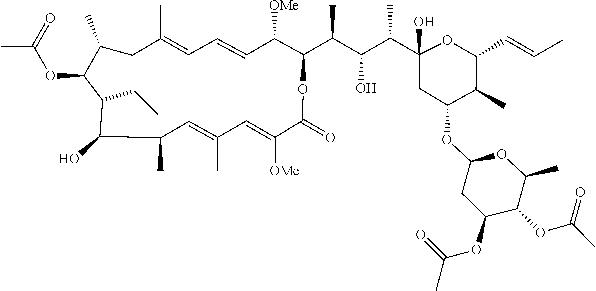 [SMILES: C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](CC)[C@@H](OC(C)=O)[C@H](C)C/C(C)=C/C=C/[C@@H]2OC)C[C@@H](O[C@@H]2C[C@H](OC(C)=O)[C@@H](OC(C)=O)[C@H](C)O2)[C@@H]1C] |
| A19 (also called S9) | 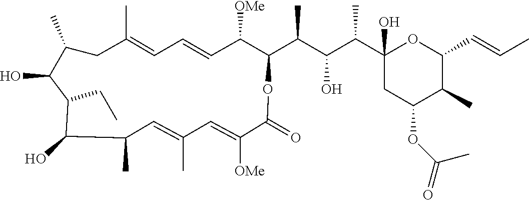 [SMILES: C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](CC)[C@@H](O)[C@H](C)C/C(C)=C/C=C/[C@@H]2OC)C[C@@H](OC(C)=O)[C@@H]1C] |
| A20 | 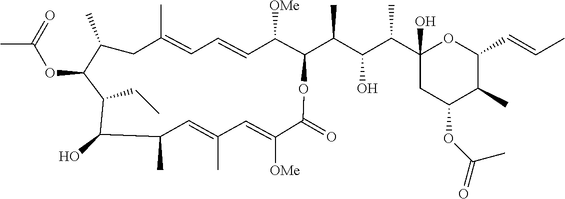 [SMILES: C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](CC)[C@@H](OC(C)=O)[C@H](C)C/C(C)=C/C=C/[C@@H]2OC)C[C@@H](OC(C)=O)[C@@H]1C] |
| A21 | 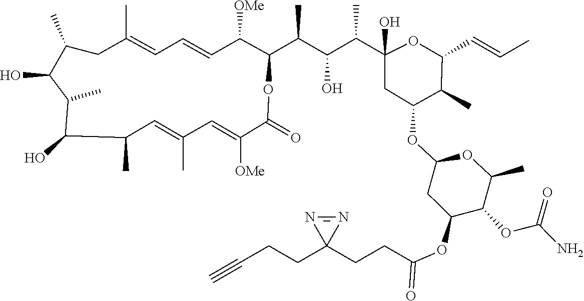 [SMILES: C#CCCC1(CCC(=O)O[C@H]2C[C@@H](O[C@@H]3C[C@](O)([C@@H](C)[C@H](O)[C@H](C)[C@H]4OC(=O)/C(OC)=C/C(C)=C/[C@@H](C)[C@@H](O)[C@@H](C)[C@@H](O)[C@H](C)C/C(C)=C/C=C/[C@@H]4OC)O[C@H](/C=C/C)[C@H]3C)O[C@@H](C)[C@@H]2OC(N)=O)N=N1] |
| A22 | 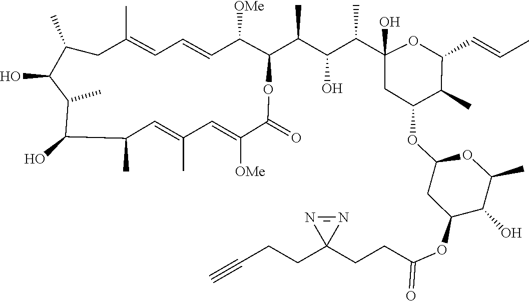 [SMILES: C#CCCC1(CCC(=O)O[C@H]2C[C@@H](O[C@@H]3C[C@](O)([C@@H](C)[C@H](O)[C@H](C)[C@H]4OC(=O)/C(OC)=C/C(C)=C/[C@@H](C)[C@@H](O)[C@@H](C)[C@@H](O)[C@H](C)C/C(C)=C/C=C/[C@@H]4OC)O[C@H](/C=C/C)[C@H]3C)O[C@@H](C)[C@@H]2O)N=N1] |

As used herein, the term “alkyl” refers to straight chained and branched saturated hydrocarbon groups containing one to eight carbon atoms, for example, one to eight carbon atoms, or one to six carbon atoms, or one to four carbon atoms. The term Cₙ means the alkyl group has “n” carbon atoms. For example, C₄ alkyl refers to an alkyl group that has 4 carbon atoms. C₁₋₈ alkyl and C₁-C₈ alkyl refer to an alkyl group having a number of carbon atoms encompassing the entire range (i.e., 1 to 8 carbon atoms), as well as all subgroups (e. g., 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, 1-2, 1, 2, 3, 4, 5, 6, 7, and 8 carbon atoms). Nonlimiting examples of alkyl groups include methyl, ethyl, n-propyl, isopropyl, n-butyl, sec-butyl (2-methylpropyl), t-butyl (1,1-dimethylethyl), 3,3-dimethylpentyl, and 2-ethylhexyl. Unless otherwise indicated, an alkyl group can be an unsubstituted alkyl group or a substituted alkyl group.

The term “haloalkyl” refers to an alkyl group as defined herein which is substituted with one or more halogen atoms, e.g., 1 to 4, 1 to 3, 1 to 2, 1, 2, 3, or 4 halogen atoms. Nonlimiting examples of haloalkyl groups include fluoromethyl, difluoromethyl, trifluoromethyl, pentafluoroethyl, 1,1-difluoroethyl, chloromethyl, chlorofluoromethyl and trichloromethyl groups.

The term “alkenyl” as used herein means a straight or branched chain hydrocarbon comprising one or more double bonds.

The term “alkynyl” as used herein means a straight or branched chain hydrocarbon comprising one or more triple bonds.

As used herein, the term “cycloalkyl” refers to an aliphatic cyclic hydrocarbon group containing five to eight carbon atoms (e.g., 5, 6, 7, or 8 carbon atoms). The term Cₙ means the cycloalkyl group has “n” carbon atoms. For example, C₅ cycloalkyl refers to a cycloalkyl group that has 5 carbon atoms in the ring. C₅₋₈ cycloalkyl and C₅-C₈ cycloalkyl refer to cycloalkyl groups having a number of carbon atoms encompassing the entire range (i.e., 5 to 8 carbon atoms), as well as all subgroups (e.g., 5-6, 6-8, 7-8, 5-7, 5, 6, 7, and 8 carbon atoms). Nonlimiting examples of cycloalkyl groups include cyclopentyl, cyclohexyl, cycloheptyl, and cyclooctyl. Unless otherwise indicated, a cycloalkyl group can be an unsubstituted cycloalkyl group or a substituted cycloalkyl group. The cycloalkyl groups described herein can be isolated or fused to another cycloalkyl group, a heterocycloalkyl group, an aryl group and/or a heteroaryl group.

As used herein, the term “heterocycloalkyl” is defined similarly as cycloalkyl, except the ring contains one to three heteroatoms independently selected from oxygen, nitrogen, and sulfur. In particular, the term “heterocycloalkyl” refers to a ring containing a total of two to 3 atoms, of which 1 or 2 of those atoms are heteroatoms independently selected from the group consisting of oxygen, nitrogen, and sulfur, and the remaining atoms in the ring are carbon atoms. Nonlimiting examples of heterocycloalkyl groups include azirines, diazirines, oxiranes, thiiranes, and the like. Heterocycloalkyl groups can be saturated or partially unsaturated ring systems. Heterocycloalkyl groups optionally can be fused to an alkyl chain as described herein, such as in a spiro configuration.

In some embodiments, R³ is a modified sugar. As used herein the term “modified sugar” refers to a sugar wherein a hydroxyl group is replaced by another group, such as an H, an alkyl group, an alkoxy group, an acyl group, a benzyl group, and the like. A sugar can be a pentose, hexose, heptose, or an amino sugar (e.g., aminopentose, aminohexose, aminoheptose, or a neuraminic acid), for example. In some embodiments, a modified sugar is a modified rhamnose or a modified 2-deoxy-β-D-rhamnose, and the like. In some cases, a modified sugar is an unnatural sugar. Nonlimiting examples of unnatural sugars include halogenated sugars (e.g., fluorinated sugars), XXX. For the avoidance of doubt, the terms “carbohydrate,” “sugar,” and “saccharide” are all used interchangeably.

As described herein, compounds described herein may optionally be substituted with one or more substituents, such as illustrated generally below, or as exemplified by particular classes, subclasses, and species described herein. It will be appreciated that the phrase “optionally substituted” is used interchangeably with the phrase “substituted or unsubstituted.” In general, the term “substituted”, whether preceded by the term “optionally” or not, refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position. When the term “optionally substituted” precedes a list, said term refers to all of the subsequent substitutable groups in that list. If a substituent radical or structure is not identified or defined as “optionally substituted”, the substituent radical or structure is unsubstituted. In some cases, the substituent is selected from group A: halo, CN, OH, CO₂H, CHO, NH₂, oxo, NO₂, C₁₋₆alkyl, C₁₋₆haloalkyl, C₁₋₆alkoxy, C₁₋₆ alkylthio, C₁₋₆ alkyl-OH, C₃₋₁₀carbocyclyl, 3-7 membered heterocyclyl, C₃₋₁₀carbocyclyl-C₁₋₆alkoxy, C₃₋₁₀carbocyclyl-O—C₁₋₆alkylene, C₃₋₁₀carbocyclyl-C₁₋₆ alkoxy-C₁₋₆alkylene, 3-7 membered heterocyclyl-C₁₋₆ alkoxy, 3-7 membered heterocyclyl-O—C₁₋₆alkylene, 3-7 membered heterocyclyl-C₁₋₆alkoxy-C₁₋₆alkylene, C₁₋₆haloalkoxy, C₁₋₆ alkoxy-C₁₋₆alkylene, C₁₋₆alkoxy-C₁₋₆alkoxy, C₁₋₆alkyl-C(O)—, C₁₋₆alkyl-C(O)O—, NHC₁₋₆alkyl, C₁₋₆alkyl-C(O)NH—, C₁₋₆ haloalkyl-C(O)NH, C₁₋₆alkyl-NHC(O)—, C₁₋₆alkyl-SO₂—, C₁₋₆alkyl-SO—, and C₁₋₆alkyISO₂NH—.

Selection of substituents and combinations of substituents contemplated herein are those that result in the formation of stable or chemically feasible compounds. The term “stable”, as used herein, refers to compounds that are not substantially altered when subjected to conditions to allow for their production, detection, and, specifically, their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week. Only those choices and combinations of substituents that result in a stable structure are contemplated. Such choices and combinations will be apparent to those of ordinary skill in the art and may be determined without undue experimentation.

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this disclosure, unless only one of the isomers is drawn specifically.

Therefore, single stereochemical isomers as well as enantiomeric, diastereomeric, cis/trans, conformational, and rotational mixtures of the present compounds are within the scope of the disclosure.

Unless otherwise indicated, all tautomeric forms of the compounds described herein are within the scope of the disclosure.

Additionally, unless otherwise indicated, structures depicted herein are also meant to include compounds that differ only in the presence of one or more isotopically enriched atoms. Discussion of an element is intended to include all isotopes of that element. For example, a substituent shown as a hydrogen includes where that hydrogen is in the deuterium or tritium isotope form, and a carbon atom can be present as a ¹³C- or ¹⁴C-carbon isotope.

It is understood that selections of values of each variable are those that result in the formation of stable or chemically feasible compounds.

### Methods of Use

The analogs described herein or pharmaceutically acceptable salts thereof can be used to modulate an HIV Nef. Modulating an HIV Nef includes inhibiting an HIV Nef.

The term “HIV Nef-associated disorder” is used herein to mean diseases or disorders whose status or progression is influenced by the expression of HIV Nef in a patient. A non-limiting example of an HIV Nef-associated disorder is HIV infection, e.g., HIV-1 infection.

As used herein, “HIV” refers to the human immunodeficiency virus. HIV includes, without limitation, HIV-1. HIV-1 includes but is not limited to extracellular virus particles and the forms of HIV-1 associated with HIV-1 infected cells. The human immunodeficiency virus (HIV) may be either of the two known types of HIV (HIV-1 or HIV-2). As used herein, HIV-1 refers to any of the known major subtypes (classes A, B, C, D, E, F, G, H, or J), outlying subtype (Group O), yet to be determined subtypes of HIV-1, and recombinations thereof.

As used herein, “HIV infection” refers to infection of a subject with HIV.

The terms, “disease”, “disorder”, and “condition” may be used interchangeably herein to refer to an HIV Nef-associated medical or pathological condition, such as HIV infection.

As used herein, the terms “subject”, “host”, and “patient” are used interchangeably. The terms “subject”, “host”, and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, or mouse) and a primate (e.g., a monkey, chimpanzee, or human), and more specifically a human. In some embodiments, the subject is a non-human animal such as a farm animal (e.g., a horse, cow, pig or sheep), or a pet (e.g., a dog, cat, guinea pig or rabbit). In a preferred embodiment, the subject is a “human”.

As used herein, the terms “treat”, “treatment,” and “treating” refer to therapeutic treatment and/or prophylactic treatments. For example, therapeutic treatments include the reduction or amelioration of the progression, severity and/or duration of HIV infection, or the amelioration of one or more symptoms (specifically, one or more discernible symptoms) of HIV infection, resulting from the administration of one or more therapies (e.g., one or more therapeutic agents such as a compound or composition described herein). In specific embodiments, the therapeutic treatment includes the amelioration of at least one measurable physical parameter of an HIV infection. In other embodiments, the therapeutic treatment includes the inhibition of the progression of an HIV infection, either physically by, e.g., stabilization of a discernible symptom, physiologically by, e.g., stabilization of a physical parameter, or both. In other embodiments, the therapeutic treatment includes the reduction or stabilization of HIV infections. Antiviral drugs can be used in the community setting to treat people who already have HIV infection to reduce the severity of symptoms and suppress the infection. Treating and HIV infection includes reducing or eliminating an HIV reservoir in a patient.

As used herein, the term “HIV reservoir” refers to a group of cells in a patient that are infected with HIV but have not produced new HIV (i.e., are in a latent stage of infection) for many months or years. Very early during acute HIV infection, a latent reservoir is established and despite effective combination anti-retroviral therapy (cART), HIV persists in latently infected cells. If a patient having a latent HIV infection stops treatment with cART, the presence of an HIV reservoir in a patient can allow an active HIV infection to become re-established in the patient.

The terms “prophylaxis”, “prophylactic”, “prophylactic use”, and “prophylactic treatment” as used herein, refer to any medical or public health procedure whose purpose is to prevent, rather than treat or cure a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a person with the disease.

As used herein, prophylactic use includes use to prevent contagion or spread of the infection in populations or individuals at high risk of HIV infection. Prophylactic use may also include treating a person who is not ill with HIV or not considered at high risk for contracting HIV, in order to reduce the chances of becoming infected with HIV and passing it on to another person.

In some embodiments, the methods of the disclosure are applied as a prophylactic measure to members of a community or population group, specifically humans, in order to prevent the spread of infection.

As used herein, an “effective amount” refers to an amount sufficient to elicit the desired biological response. In the present disclosure the desired biological response is to inhibit the replication of HIV, to reduce the amount of HIV, or to reduce or ameliorate the severity, duration, progression, or onset of an HIV infection, prevent the advancement of an HIV infection, prevent the recurrence, development, onset or progression of a symptom associated with an HIV infection, or enhance or improve the prophylactic or therapeutic effect(s) of another therapy used against HIV infections. The precise amount of compound administered to a subject will depend on the mode of administration, the type and severity of the infection and on the characteristics of the subject, such as general health, age, sex, body weight and tolerance to drugs. The skilled artisan will be able to determine appropriate dosages depending on these and other factors. When co-administered with other antiviral agents, e.g., when co-administered with an anti-HIV medication, an effective amount of the second agent will depend on the type of drug used. A safe amount is one with minimal side effects, as can readily be determined by those skilled in the art. Suitable dosages are known for approved agents and can be adjusted by the skilled artisan according to the condition of the subject, the type of condition(s) being treated and the amount of a compound described herein being used. In cases where no amount is expressly noted, a safe and effective amount should be assumed. For example, compounds described herein can be administered to a subject in a dosage range from between approximately 0.01 to 100 mg/kg body weight/day for therapeutic or prophylactic treatment.

As used herein, a “safe and effective amount” of a compound or composition described herein is an effective amount of the compound or composition which does not cause excessive or deleterious side effects in a patient.

Generally, dosage regimens can be selected in accordance with a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the renal and hepatic function of the subject; and the particular compound or salt thereof employed, the duration of the treatment; drugs used in combination or coincidental with the specific compound employed, and like factors well known in the medical arts. The skilled artisan can readily determine and prescribe a safe and effective amount of the compounds described herein required to treat, to prevent, inhibit (fully or partially) or arrest the progress of the disease.

Dosages of the compounds described herein can range from between about 0.01 to about 100 mg/kg body weight/day, about 0.01 to about 50 mg/kg body weight/day, about 0.1 to about 50 mg/kg body weight/day, or about 1 to about 25 mg/kg body weight/day. It is understood that the total amount per day can be administered in a single dose or can be administered in multiple dosing, such as twice a day (e.g., every 12 hours), three times a day (e.g., every 8 hours), or four times a day (e.g., every 6 hours).

For therapeutic treatment, the compounds described herein can be administered to a patient within, for example, 48 hours (or within 40 hours, or less than 2 days, or less than 1.5 days, or within 24 hours) of onset of symptoms (e.g., nasal congestion, sore throat, cough, aches, fatigue, headaches, and chills/sweats). The therapeutic treatment can last for any suitable duration, for example, for 5 days, 7 days, 10 days, 14 days, etc.

### Pharmaceutically Acceptable Salts

The analogs described herein can exist in free form, or, where appropriate, as salts. Those salts that are pharmaceutically acceptable are of particular interest since they are useful in administering the analogs described below for medical purposes. Salts that are not pharmaceutically acceptable are useful in manufacturing processes, for isolation and purification purposes, and in some instances, for use in separating stereoisomeric forms of the compounds described herein or intermediates thereof.

As used herein, the term “pharmaceutically acceptable salt” refers to salts of a compound which are, within the scope of sound medical judgment, suitable for use in contact with the tissues of humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the analogs described herein include those derived from suitable inorganic and organic acids and bases. These salts can be prepared in situ during the final isolation and purification of the compounds.

Where the compound described herein contains a basic group, or a sufficiently basic bioisostere, acid addition salts can be prepared by 1) reacting the purified compound in its free-base form with a suitable organic or inorganic acid and 2) isolating the salt thus formed. In practice, acid addition salts might be a more convenient form for use and use of the salt amounts to use of the free basic form.

Examples of pharmaceutically acceptable, non-toxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Where the compound described herein contains a carboxylic acid group or a sufficiently acidic bioisostere, base addition salts can be prepared by 1) reacting the purified compound in its acid form with a suitable organic or inorganic base and 2) isolating the salt thus formed. In practice, use of the base addition salt might be more convenient and use of the salt form inherently amounts to use of the free acid form. Salts derived from appropriate bases include alkali metal (e.g., sodium, lithium, and potassium), alkaline earth metal (e.g., magnesium and calcium), ammonium and N⁺(C₁₋₄alkyl)₄ salts. This disclosure also envisions the quaternization of any basic nitrogen-containing groups of the compounds disclosed herein. Water or oil-soluble or dispersible products may be obtained by such quaternization.

Basic addition salts include pharmaceutically acceptable metal and amine salts. Suitable metal salts include the sodium, potassium, calcium, barium, zinc, magnesium, and aluminum. The sodium and potassium salts are usually preferred. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which are frequently used in medicinal chemistry because of their low toxicity and acceptability for medical use. Ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, dicyclohexylamine and the like.

Other acids and bases, while not in themselves pharmaceutically acceptable, may be employed in the preparation of salts useful as intermediates in obtaining the compounds described herein and their pharmaceutically acceptable acid or base addition salts.

It should be understood that this disclosure includes mixtures/combinations of different pharmaceutically acceptable salts and also mixtures/combinations of compounds in free form and pharmaceutically acceptable salts.

### Pharmaceutical Compositions

The analogs described herein can be formulated into pharmaceutical compositions that further comprise a pharmaceutically acceptable carrier, diluent, adjuvant or vehicle. In some embodiments, the present disclosure relates to a pharmaceutical composition comprising an analog described herein, and a pharmaceutically acceptable carrier, diluent, adjuvant or vehicle. In some embodiments, the present disclosure includes a pharmaceutical composition comprising a safe and effective amount of a compound described herein or a pharmaceutically acceptable salt thereof and a pharmaceutically acceptable carrier, diluent, adjuvant or vehicle. Pharmaceutically acceptable carriers include, for example, pharmaceutical diluents, excipients or carriers suitably selected with respect to the intended form of administration, and consistent with conventional pharmaceutical practices.

An “effective amount” includes a “therapeutically effective amount” and a “prophylactically effective amount”. The term “therapeutically effective amount” refers to an amount effective in treating and/or ameliorating an HIV infection in a patient.

A pharmaceutically acceptable carrier may contain inert ingredients which do not unduly inhibit the biological activity of the compounds. The pharmaceutically acceptable carriers should be biocompatible, e.g., non-toxic, non-inflammatory, non-immunogenic or devoid of other undesired reactions or side-effects upon the administration to a subject. Standard pharmaceutical formulation techniques can be employed.

The pharmaceutically acceptable carrier, adjuvant, or vehicle, as used herein, includes any solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds described herein, such as by producing any undesirable biological effect or otherwise interacting in a deleterious manner with any other component(s) of the pharmaceutically acceptable composition, its use is contemplated to be within the scope of this disclosure. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful or uncomfortable or risky. Side effects include, but are not limited to fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

Some examples of materials which can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

### Administration Methods

The analogs and pharmaceutically acceptable compositions described above can be administered to humans and other animals orally, rectally, parenterally, intracisternally, intravaginally, intraperitoneally, topically (as by powders, ointments, or drops), bucally, as an oral or nasal spray, to the pulmonary system, such as by using an inhaler, such as a metered dose inhaler (MDI), or the like, depending on the severity of the infection being treated.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of an analog described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are specifically suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The compositions described herein may be administered orally, parenterally, by inhalation spray, topically, rectally, nasally, buccally, vaginally or via an implanted reservoir. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Specifically, the compositions are administered orally, intraperitoneally or intravenously.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as polysorbates, sorbitan esters, and other emulsifying agents or bioavailability enhancers which are commonly used in the manufacture of pharmaceutically acceptable solid, liquid, or other dosage forms may also be used for the purposes of formulation.

The pharmaceutical compositions described herein may be orally administered in any orally acceptable dosage form including, but not limited to, capsules, tablets, aqueous suspensions or solutions. In the case of tablets for oral use, carriers commonly used include, but are not limited to, lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions described herein may be administered in the form of suppositories for rectal administration. These can be prepared by mixing the agent with a suitable non-irritating excipient which is solid at room temperature but liquid at rectal temperature and therefore will melt in the rectum to release the drug. Such materials include, but are not limited to, cocoa butter, beeswax and polyethylene glycols.

The pharmaceutical compositions described herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds described herein include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

For ophthalmic use, the pharmaceutical compositions may be formulated as micronized suspensions in isotonic, pH adjusted sterile saline, or, specifically, as solutions in isotonic, pH adjusted sterile saline, either with or without a preservative such as benzalkonium chloride. Alternatively, for ophthalmic uses, the pharmaceutical compositions may be formulated in an ointment such as petrolatum.

The compounds for use in the methods described herein can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

The disclosure will be more fully understood by reference to the examples described herein which detail exemplary embodiments. These examples should not, however, be construed as limiting the scope of the disclosure. All citations throughout the disclosure are hereby expressly incorporated by reference.

### COMPARATIVE EXAMPLES

Several compounds were synthesized for comparative purposes with the compounds of Formula (I).

### Comparative Example 1: Structure-Activity Relationship (SAR) Study

The identification of a potent Nef inhibitor that enhances CTL-mediated clearance of active and latent reservoirs of HIV is an important goal toward developing a cure. Through the curation and testing of a SAR panel with 74 macrocycles, it was determined that microbially derived 16-, 18-, or 24-membered ring compounds are potent Nef inhibitors. Prior to the discovery of the plecomacrolides as potent Nef inhibitors, compounds 1 and 2 were primarily known for their highly specific and potent V-ATPase inhibitory activity in eukaryotic cells (Bowman et al., 1988; Osteresch et al., 2012; Painter et al., 2020). Moreover, the 24-membered ring macrocycle archazolid A (3) that potently inhibits Nef is also an effective V-ATPase inhibitor (Osteresch et al., 2012). Recently, cryo-EM structures revealed that compounds 2 and 3 have distinct binding sites on the V₀ domain of V-ATPase. Without wishing to be bound by any particular theory, the C-7-OH of 2 and 3 forms a critical hydrogen bond with a tyrosine residue that mediates binding to the c-subunit (Keon et al., 2022). In the instant disclosure, a semi-synthetic derivative of 2 that has been oxidized at C-7-OH to a keto group (S17) led to abrogation of activity at all concentrations tested, highlighting the role of the hydrogen bond donating abilities of the C-7-OH to achieve Nef and V-ATPase inhibition. Without wishing to be bound by any particular theory, it appears that Nef-mediated MHC-1 downmodulation is activated through a V-ATPase-dependent step. However, currently, there is no structure available detailing how 1 induces V-ATPase inhibition, only a hypothesis that it binds the c-subunit similarly to its 16-membered ring family member, 2 (Osteresch et al., 2012; Yoshimori et al., 1991). The SAR study disclosed herein highlights how 1, and its semi-synthetic derivatives act differently than 2 or 3 by inhibiting Nef at low concentrations with minimal effect on lysosomal neutralization. This ability to separate Nef inhibition while limiting V-ATPase inhibition supports the hypothesis that distinct pathways exist for downmodulation of MHC-1 and V-ATPase dependent lysosomal acidification (Table B). To visualize these two inhibitory pathways, the neutralization window (the fold-change between Nef and V-ATPase IC₅₀s) between the parent compounds (CMA/CMB) was compared with that for their respective semi-synthetic derivatives with the aim of developing an improved candidate for future in vivo studies.

One of the most important factors for determining potency of Nef and V-ATPase inhibition was macrolactone ring-size. Comparing 16- versus 18-membered-ring plecomacrolides (2 vs. 1) revealed that the latter has 100-fold greater Nef inhibitory activity. In addition, the 24-membered macrolactone (3) had potent Nef inhibitory activity, however others such as rifampicin, did not, indicating that sub-nM Nef inhibitory activity is not a general feature of large macrocyclic compounds (Table B). Smaller rings with 10-, 12-, and 14-membered macrolactones failed to display inhibitory activity at all concentrations tested (Table B). For molecules 1-3, the trend observed for Nef IC₅₀ corresponded to that observed for lysosomal neutralization (1<3<2; Table B). However, there were distinct differences in the neutralization window (9.4-, 3.6-, & 2.9-fold for 1, 3, & 2, respectively) supporting the hypothesis that alternative pathways are employed to inhibit Nef-dependent MHC-I downmodulation versus lysosomal acidification for each of these compounds. Without wishing to be bound by any particular theory, the level of potency for lysosomal neutralization may relate to differential binding to V-ATPase based on ring size and other structural feature. Indeed, previous studies have noted that the size of the plecomacrolide macrocycle impacts binding orientation to the c-subunit of V-ATPase (Osteresch et al., 2012; Wang et al., 2021). Identification of the direct target and structural studies are required to explain the relative potencies of 1-3 for Nef inhibition.

In addition to the macrolactone ring size, it was found that the pyran ring is important for both Nef and V-ATPase inhibition, as conversion to a linear system drastically reduced both inhibitory activities (2 vs. S8). Within the pyran ring, modifications at C-19 of 2 had little effect (S5), whereas alterations at C-21 of 2 either increased or decreased potency (Table B). For example, acetylation (S9) improved potency for both inhibitory activities whereas the addition of longer acyl chains (S10, S11) had an opposing effect. We similarly observed both positive and negative effects on potency for changes at C-21 of 19-deoxy bafilomycin (S5). Conversion of C-21-OH to an allyl-ester (S27) decreased potency whereas addition of a longer chain with a terminal alkyne improved potency (S65, Table B). This trend was reinforced by the C-21-penta-3-enoate (S29) and C-21-penta-2,4-dienoate (S12) derivatives of 2 where addition of acyl chains bearing variant degrees of unsaturation improved potency compared to S10 or S11 (Table B). Without wishing to be bound by any particular theory, it is possible that hydrophobic interactions mediated by these linear substituents may be important for target binding especially because they parallel the effects of hydrophobic substituents previously reported to play a role in V-ATPase binding (Wang et al., 2021).

CMA (1) contains a 4′-carbamoyl-β-D-rhamnose moiety at the pyranyl C-23 position that is absent from the analogous C-21 position of 2. Two microbially derived analogs, S14 and S15 were tested as they contained modified β-D-rhamnose moieties (Table B). When C-21-OH is glycosylated, the 16-membered scaffold becomes a significantly more potent Nef inhibitor with single-digit nM IC₅₀s (S14 and S15; Table B). However, neutralization and therapeutic windows were relatively poor. Thus, although identifying semi-synthetic derivatives of 2 have helped guide synthetic modification strategies, the bafilomycin system has largely been ruled out as a lead scaffold for generating improved HIV Nef inhibitor drug candidates.

The same trend was observed with 18-membered ring plecomacrolides as comparison of Nef IC₅₀s amongst concanamycin F (6) and concanamycin C (5) revealed that the β-D-rhamnose moiety (6 vs 5) improved potency (Table B). Addition of the 4′-carbamoyl-β-D-rhamnose moiety onto the pyran ring (5 vs. 1) led to a 14-fold improvement in potency (Table B). This was further supported by the testing of an additional aglycone of concanamycin B [concanamycin X (7)], lacking the 4′-carbamoyl-β-D-rhamnose, which also suffered a reduction in potency (Table B). It is also important to note that both aglycones displayed significantly smaller neutralization windows compared to their parent compounds, especially for 4. These results confirm, using the 18-membered ring plecomacrolide scaffold, that the β-D-rhamnose and 4′-carbamoyl moieties are integral motifs for nM inhibition of HIV Nef and inhibition of V-ATPase.

For both 1 and 2, it was observed that the pyran ring is vital for Nef inhibition and lysosomal neutralization, as conversion to a linear system (S8) drastically reduced both inhibitory activities for 2. More limited changes, including modification at C-19 of 2 reduced Nef inhibitory potency while other changes, such as esterification of C-21-OH of 2 yielded results that depended on chain length [small acyl chains (S9) improved potency for both inhibitory activities whereas the addition of longer acyl chains (S10, S11) had the opposite effect]. It was similarly observed both positive and negative effects on potency for changes at C-21 of 19-deoxy bafilomycin (S5); conversion of C-21-OH to an allyl-ester (S27) decreased potency whereas addition of a longer chain with a terminal alkyne improved potency (S28). This trend was reinforced by the C-21-penta-3-enoate (S29) and C-21-penta-2,4-dienoate (S12) derivatives of 2 where addition of acyl chains bearing variant degrees of unsaturation improved potency compared to S10 or S11. Without wishing to be bound by any particular theory, it is thought that hydrophobic interactions mediated by these linear substituents may be important for target binding especially because they parallel the effects of hydrophobic substituents previously reported to play a role in V-ATPase bindingfu.

The key results of the instantly-disclosed study reveal that concanamycin B (4), which bears a C-8 methyl compared to ethyl in 1 represents the most compelling starting point to identify lead compounds for clinical development. Although 4 has 10-fold lower potency for Nef inhibition compared to 1, it has an expanded neutralization window (approximately 24-fold vs 10-fold for 1: Table B). Further semi-synthetic modification of 4 revealed that compound 13 (3′,9-diacetylated concanamycin B) demonstrated both an expanded therapeutic and neutralization window and has good overall characteristics for further development (Table B). Expansion of the neutralization window was not observed with a single acetylation at 3′-OH (12), highlighting that the C-9 position on 4 is critical for distinguishing Nef versus V-ATPase inhibitory activity.

The most compelling 18-membered macrolactone derivatives were obtained by esterifying the C-9-OH position, and motivated synthesis of acetyl and diacetyl derivatives 14/15, S22/S23, and S20/S21 (derived from 1, 5, and 6, respectively). The most notable compound from the acetylation panel was 3′,9-diacetyl CMA (15), as it displayed a significantly increased neutralization window of the CMA scaffold, approaching neutralization windows that had only been observed with CMB and its derivatives, while maintaining both potent Nef inhibition and the therapeutic window. The increase in magnitude of the neutralization window for analogs of 1 and therapeutic windows for 4 when esterifying C-9-OH (which was observed to a lesser degree in the mono-acetylated compounds (14 & 12)), suggests that diversifying C-9 is critical for distinguishing Nef inhibition from lysosomal acidification and cellular toxicity. Further studies are needed to assess whether improving the neutralization window in this manner will also reduce toxicity in a more significant way.

Through this SAR study it was shown that 18-membered plecomacrolides with an intact pyran ring system, a C-8 methyl group, 4′-carbamoyl-β-D-rhamnose, and addition of short acyl ester chains represent good opportunities for identifying improved Nef inhibitors. Modifications to the macrocycle, the pyran ring, or substituents off the ring impact potencies in both 16- and 18-membered ring compounds and further sugar diversification should be considered moving forward to gain a deeper understanding of the role this key moiety plays in nM potency and the mechanism of action. Differences in the way these compounds inhibit Nef-dependent MHC-I downmodulation versus lysosomal neutralization suggest there are structural parameters in the respective molecule(s) for each inhibitory pathway that remain to be identified.

### Comparative Example 2: Synthesis of Bafilomycin A₂ (Compound 1, Also Called S4)

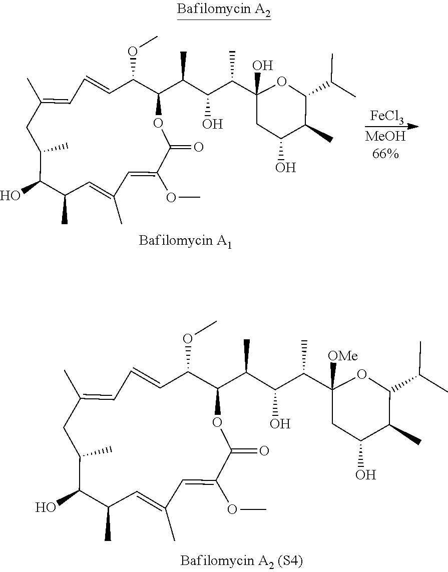

Bafilomycin A₁ (74 mg, 0.090 mmol) was dissolved in MeOH (2 mL) under anhydrous conditions and cooled to 0° C. FeCl₃ (2.9 mg, 0.018 mmol, 0.2 eq) was added to the solution. The FeCl₃ was added to the reaction after first dissolving it in a separate flask with MeOH. The reaction was ran at room temperature for 15-30 minutes, checking TLC (55% ethyl acetate/hexane) for consumption of starting material. The reaction was quenched with phosphate buffer (1.0 M, 7.1 pH). The reaction was dissolved in DCM, washed with water, brine, dried over MgSO₄, filtered, and concentrated. The crude reaction was then purified by flash column chromatography (30-60% ethyl acetate/hexane) to yield the product. S4 ¹H NMR (599 MHz, Acetone) δ 6.69 (d, J=0.9 Hz, 1H), 6.66 (dd, J=15.0, 10.8 Hz, 1H), 5.96 (dt, J=8.9, 1.2 Hz, 1H), 5.81 (d, J=10.8 Hz, 1H), 5.17 (dd, J=15.0, 9.0 Hz, 1H), 5.09 (dd, J=8.1, 1.5 Hz, 1H), 4.05 (d, J=8.5 Hz, 1H), 4.04 (d, J=5.3 Hz, 1H), 3.93-3.89 (m, 1H), 3.66 (s, 3H), 3.52-3.49 (m, 1H), 3.46 (q, J=4.1, 3.1 Hz, 1H), 3.32 (dp, J=8.2, 3.0, 2.5 Hz, 1H), 3.23 (s, 3H), 3.11-3.05 (m, 1H), 3.04 (s, 3H), 2.59-2.46 (m, 1H), 2.19 (dd, J=13.3, 4.3 Hz, 1H), 2.12-2.06 (m, 1H), 2.05 (p, J=2.2 Hz, 2H), 2.04-1.99 (m, 1H), 1.98 (d, J=1.2 Hz, 3H), 1.97-1.93 (m, 1H), 1.92 (d, J=1.4 Hz, 3H), 1.88 (p, J=7.1 Hz, 1H), 1.48 (dd, J=13.4, 10.6 Hz, 1H), 1.27 (dd, J=10.0, 6.6 Hz, 1H), 1.08 (dd, J=14.1, 7.2 Hz, 1H), 1.05 (d, J=7.0 Hz, 3H), 1.03 (d, J=6.9 Hz, 3H), 0.97 (d, J=7.0 Hz, 3H), 0.95 (d, J=6.9 Hz, 3H), 0.92 (t, J=7.0 Hz, 6H), 0.89 (d, J=6.8 Hz, 3H). ¹³C NMR (151 MHz, Acetone) δ 167.23, 145.31, 144.50, 142.13, 134.06, 133.61, 132.68, 127.27, 125.32, 103.84, 83.84, 80.45, 78.18, 77.65, 70.39, 70.34, 60.19, 55.72, 46.68, 42.32, 41.52, 41.24, 40.06, 39.84, 39.17, 38.05, 29.14, 22.40, 21.12, 20.21, 17.80, 14.63, 14.09, 12.63, 11.11, 7.90.

### Comparative Example 3: Synthesis of 19-deoxy Bafilomycin (S5)

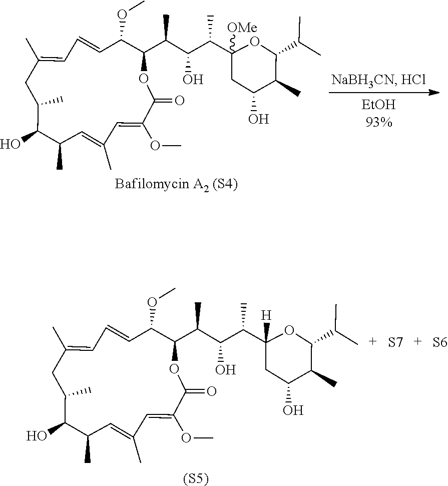

Bafilomycin A₂ (45 mg, 70.7 μmol, was dissolved in EtOH (6 mL) and stirred under nitrogen. NaBH₃CN (26.6 mg, 424 μmol, 6.0 eq) and HCl (11.1 mg, 304 μmol, 4.3 eq) were added to the reaction flask that was then left to run for approximately 1 hour, checking by TLC to determine the reaction's completeness. The crude reaction mixture was extracted with DCM (×4). The organics were combined, washed with water, brine, dried over NaSO₄, filtered and concentrated. The crude material was purified further by flash column chromatography (12-100% ethyl acetate/hexane) to yield S5 as the major product (40. mg, 70.7 μmol, 93%) as a pale-yellow solid, with small amounts of two minor products, 19-Deoxy-20-ene Bafilomycin A₁ (S7) and 19-Deoxy-21-Methoxy Bafilomycin A₁ (S6). 19-deoxy Bafilomycin ¹H NMR (599 MHz, Acetone) δ 6.68 (s, 1H), 6.64 (dd, J=15.0, 10.8 Hz, 1H), 5.94 (d, J=8.9 Hz, 1H), 5.80 (d, J=10.8 Hz, 1H), 5.22-5.18 (m, 1H), 5.17 (dd, J=14.6, 8.4 Hz, 1H), 4.04 (t, J=8.3 Hz, 1H), 4.00 (d, J=5.4 Hz, 1H), 3.81-3.76 (m, 1H), 3.64 (t, J=4.1 Hz, 1H), 3.62 (s, 3H), 3.37 (ddd, J=11.5, 8.2, 1.8 Hz, 1H), 3.32 (s, 1H), 3.27 (td, J=10.2, 4.6 Hz, 1H), 3.23 (s, 3H), 2.79 (dd, J=10.0, 2.2 Hz, 1H), 2.60 (s, 1H), 2.54 (ddd, J=9.0, 7.0, 2.0 Hz, 1H), 2.14 (s, 1H), 2.07-2.00 (m, 3H), 1.97 (d, J=1.3 Hz, 3H), 1.90 (s, 3H), 1.89-1.82 (m, 2H), 1.69-1.61 (m, 1H), 1.26 (s, 1H), 1.15 (q, J=11.5 Hz, 1H), 1.05 (d, J=7.1 Hz, 3H), 0.95 (d, J=6.8 Hz, 3H), 0.93 (d, J=6.9 Hz, 3H), 0.91 (d, J=6.5 Hz, 3H), 0.87 (d, J=6.9 Hz, 3H), 0.84 (d, J=7.0 Hz, 3H), 0.79 (d, J=6.8 Hz, 3H). ¹³C NMR (151 MHz, Acetone) δ 166.63, 144.72, 144.14, 142.34, 133.80, 133.15, 132.64, 127.27, 125.35, 85.14, 84.03, 80.50, 77.68, 77.62, 74.22, 70.45, 59.97, 55.70, 42.31, 42.06, 41.26, 40.83, 40.71, 38.76, 38.12, 29.23, 22.52, 21.58, 20.06, 17.91, 14.83, 14.19, 12.67, 10.64, 8.89.

### Comparative Example 4: Synthesis of 19-Deoxy-20-ene Bafilomycin A1 (S7)

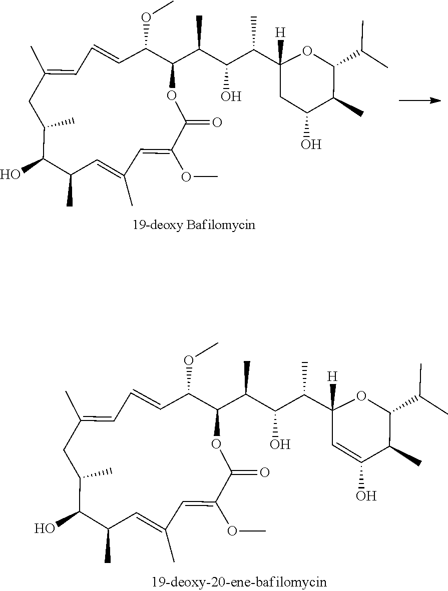

¹H NMR (599 MHz, acetone) δ 6.66 (d, J=0.9 Hz, 1H), 6.63 (dd, J=15.1, 10.9 Hz, 1H), 5.94 (dt, J=8.9, 1.2 Hz, 1H), 5.83-5.79 (m, 2H), 5.62 (dt, J=10.3, 2.1 Hz, 1H), 5.24 (dd, J=7.4, 2.0 Hz, 1H), 5.20 (dd, J=15.1, 8.6 Hz, 1H), 4.07-4.00 (m, 3H), 3.77 (ddd, J=10.3, 3.8, 1.7 Hz, 1H), 3.64 (s, 1H), 3.62 (d, J=3.8 Hz, 1H), 3.33 (ddd, J=7.6, 5.7, 2.0 Hz, 1H), 3.22 (s, 3H), 2.95 (dd, J=9.5, 2.1 Hz, 1H), 2.53 (tt, J=7.9, 6.0 Hz, 1H), 2.28-2.20 (m, OH), 2.09 (dd, J=14.1, 11.3 Hz, 1H), 2.04-1.99 (m, 1H), 1.97 (d, J=1.3 Hz, 3H), 1.92-1.85 (m, 5H), 1.74-1.68 (m, 1H), 1.05 (d, J=7.0 Hz, 3H), 1.01 (d, J=6.9 Hz, 3H), 0.94 (dd, J=7.9, 6.9 Hz, 6H), 0.88 (d, J=7.1 Hz, 3H), 0.86 (d, J=6.9 Hz, 2H), 0.83 (d, J=6.8 Hz, 3H). ¹³C NMR (151 MHz, acetone) δ 166.20, 144.43, 143.88, 142.45, 133.58, 133.33, 132.76, 132.53, 129.34, 127.16, 125.39, 85.05, 84.13, 80.48, 78.72, 77.24, 71.12, 59.91, 55.74, 42.32, 41.04, 39.91, 38.89, 38.22, 32.26, 22.71, 21.11, 19.85, 18.02, 17.43, 15.21, 14.18, 10.73, 8.97.

### Comparative Example 5: 19-Deoxy-21-Methoxy B Bafilomycin A1 (S6)

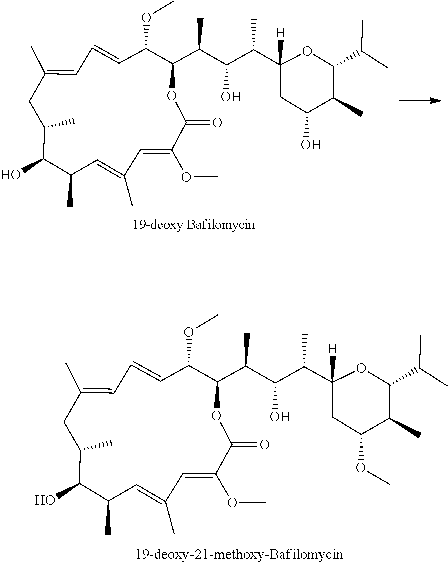 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@H]2C[C@@H](O)[C@H](C)[C@@H](C(C)C)O2)OC1=O ; CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@H]2C[C@@H](OC)[C@H](C)[C@@H](C(C)C)O2)OC1=O]

¹H NMR (599 MHz, acetone) δ 6.68 (d, J=0.9 Hz, 1H), 6.64 (dd, J=15.0, 10.8 Hz, 1H), 5.94 (dt, J=9.0, 1.2 Hz, 1H), 5.81 (d, J=10.8 Hz, 1H), 5.20-5.14 (m, 2H), 4.03 (dd, J=10.4, 6.8 Hz, 2H), 3.78 (ddd, J=10.4, 4.5, 1.7 Hz, 1H), 3.66 (d, J=4.5 Hz, 1H), 3.62 (s, 3H), 3.37-3.30 (m, 5H), 3.23 (s, 3H), 2.92 (td, J=10.4, 4.6 Hz, 1H), 2.82-2.80 (m, 1H), 2.54 (dqd, J=9.0, 7.0, 1.9 Hz, 1H), 2.27 (ddd, J=12.1, 4.6, 1.9 Hz, 1H), 1.97 (d, J=1.2 Hz, 3H), 1.90 (d, J=1.3 Hz, 3H), 1.86 (dtt, J=13.8, 6.9, 3.8 Hz, 1H), 1.67 (tt, J=7.3, 5.6 Hz, 1H), 1.33 (dtd, J=9.9, 6.5, 3.5 Hz, 1H), 1.05 (d, J=7.0 Hz, 3H), 0.95 (d, J=6.8 Hz, 3H), 0.92 (d, J=6.9 Hz, 3H), 0.88-0.84 (m, 8H), 0.78 (d, J=6.8 Hz, 3H). ¹³C NMR (151 MHz, acetone) δ 166.67, 144.78, 144.19, 142.31, 133.84, 133.18, 132.64, 127.25, 125.33, 85.09, 83.98, 83.73, 80.47, 77.63, 77.48, 70.41, 59.95, 56.16, 55.69, 42.28, 41.30, 40.95, 40.10, 38.72, 38.09, 35.57, 22.48, 21.51, 20.09, 17.89, 14.74, 14.19, 12.61, 10.61, 8.86.

### Comparative Example 6: Synthesis of 19-Deoxy-21-allylester Bafilomycin A1 (S42)

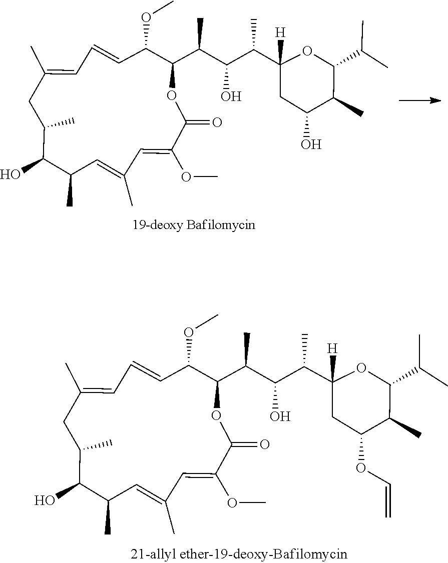 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@H]2C[C@@H](O)[C@H](C)[C@@H](C(C)C)O2)OC1=O ; C=CO[C@@H]1C[C@H]([C@@H](C)[C@H](O)[C@H](C)[C@H]2OC(=O)/C(OC)=C/C(C)=C/[C@@H](C)[C@@H](O)[C@@H](C)C/C(C)=C/C=C/[C@@H]2OC)O[C@H](C(C)C)[C@H]1C]

To a solution of S5 (7.4 mg, 12 μmol) in DCM (2 mL) was added crotonic anhydride (4.7 mg, 30 μmol, 2.5 eq.) and DBU (5.5 μL, 36 μmol, 3 eq.) at 0° C. The reaction was allowed to warm to room temperature and proceed for 16 h. The reaction was diluted to −20 mL with DCM and washed with an equal volume of a 3% (w/v) citric acid solution, followed by PBS buffer (pH 7.4) and brine. The organic layer was dried in vacuo and dissolved in HPLC acetone (300 μL). Purification was accomplished using a semi-preparative reversed phase C18 (2) column (Phenomenex, 250×10 mm), running a gradient of water (solvent A) and acetonitrile (solvent B) at 8 mLmin-1. The gradient was run using an initial 60% Solvent B isocratic step for 2 min, followed by a linear increasing gradient from 60% to 95% solvent B over 38 min (40 min total). This was followed by a second isocratic step using 95% solvent B for 20 min (60 min total). The compound of interest eluted at ~45.5 min, and dried in vacuo yielding 3.2 mg (41%) of a white solid. Subsequent NMR characterization identified the compound as 19-deoxy bafilomycin A1 allyl ester, suggesting a spontaneous conversion from the crotonyl ester to the allyl ester during derivatization. ¹H NMR (599 MHz, acetone) δ 6.68 (d, J=0.9 Hz, 1H), 6.64 (dd, J=15.0, 10.8 Hz, 1H), 6.03-5.86 (m, 2H), 5.80 (d, J=10.8 Hz, 1H), 5.21-5.10 (m, 4H), 4.63 (td, J=10.7, 4.8 Hz, 1H), 4.04 (d, J=8.4 Hz, 1H), 4.02 (d, J=5.1 Hz, 1H), 3.77 (dtd, J=9.0, 4.5, 1.6 Hz, 1H), 3.70 (d, J=4.6 Hz, 1H), 3.63 (s, 3H), 3.45 (ddd, J=11.5, 8.3, 1.9 Hz, 1H), 3.32 (ddd, J=7.4, 5.7, 2.0 Hz, 1H), 3.23 (s, 3H), 3.17-3.08 (m, 2H), 2.94 (dd, J=10.0, 2.2 Hz, 1H), 2.54 (tt, J=8.1, 6.1 Hz, 1H), 2.13 (ddd, J=12.1, 4.8, 2.0 Hz, 1H), 1.97 (d, J=1.2 Hz, 3H), 1.91 (d, J=1.2 Hz, 3H), 1.88 (qd, J=6.8, 5.7, 3.8 Hz, 2H), 1.72-1.66 (m, 1H), 1.56 (tq, J=10.2, 6.5 Hz, 1H), 1.19 (q, J=11.5 Hz, 1H), 1.05 (d, J=7.0 Hz, 3H), 0.97 (d, J=6.9 Hz, 3H), 0.93 (d, J=6.9 Hz, 3H), 0.87 (d, J=6.9 Hz, 3H), 0.83 (d, J=7.1 Hz, 3H), 0.81 (dd, J=6.7, 3.4 Hz, 6H). ¹³C NMR (151 MHz, acetone) δ 171.29, 166.72, 144.83, 144.23, 142.29, 133.88, 133.23, 132.66, 132.02, 127.25, 125.33, 118.28, 84.74, 83.95, 80.48, 77.63, 77.22, 77.20, 70.36, 59.98, 55.69, 42.29, 41.33, 40.83, 39.74, 39.02, 38.69, 38.09, 36.72, 29.18, 22.47, 21.40, 20.11, 17.88, 14.67, 14.19, 12.53, 10.58, 8.68.

### Comparative Example 7: 19-Deoxy-21(4-Pentynoate) Bafilomycin A1 (S43)

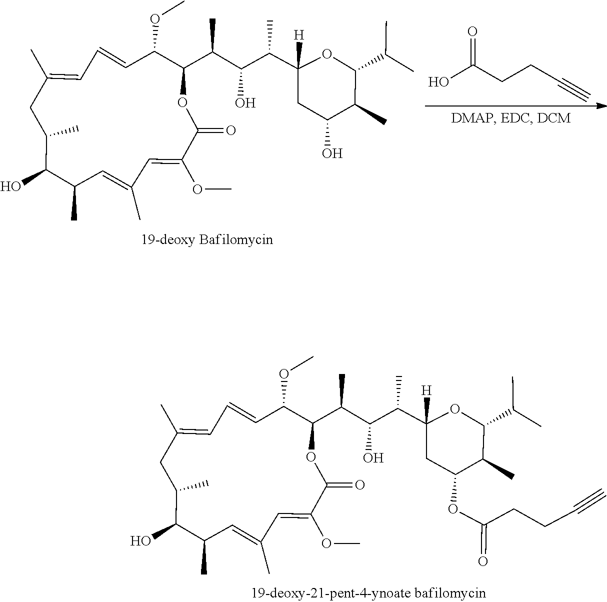

In a flame dried flask, S5 (7.9 mg, 13 mmol), pentynoic acid (1.9 mg, 20. mmol, 1.5 eq), and N,N′-dicyclohexylcarbodiimide (DCC) (4.0 mg, 20. mmol, 1.5 eq) was dissolved in dry DCM (260 μL, 0.05 M) at 0° C. DMAP was added to the solution (tip of spatula approx. 5 mol %) before the reaction was allowed to warm to RT and run for 24 hrs. The reaction was washed with sat. NaHCO₃, back extracted with DCM (1 mL×3), dried over Na2SO4, filtered, and concentrate in vacuo to give 8.0 mg of material. The material was purified by RP HPLC to yield 2.5 mg (3.6 mmol, 28%) of product. ¹H NMR (700 MHz, acetone) δ 6.68 (s, 1H), 6.64 (dd, J=15.0, 10.8 Hz, 1H), 5.94 (d, J=8.9 Hz, 1H), 5.80 (d, J=10.8 Hz, 1H), 5.21-5.14 (m, 2H), 4.66 (td, J=10.7, 4.8 Hz, 1H), 4.03 (t, J=8.4 Hz, 1H), 4.00 (d, J=5.6 Hz, 1H), 3.81-3.75 (m, 1H), 3.69 (d, J=4.6 Hz, 1H), 3.63 (s, 3H), 3.45 (ddd, J=10.6, 8.4, 1.9 Hz, 1H), 3.32 (p, J=3.3, 2.6 Hz, 1H), 3.23 (s, 3H), 2.94 (dd, J=10.0, 2.2 Hz, 1H), 2.62-2.51 (m, 3H), 2.49 (td, J=6.8, 2.0 Hz, 2H), 2.38 (t, J=2.6 Hz, 1H), 2.13 (ddd, J=12.1, 4.9, 1.9 Hz, 1H), 1.97 (d, J=1.3 Hz, 3H), 1.90 (s, 3H), 1.89-1.85 (m, 2H), 1.69 (p, J=7.3 Hz, 1H), 1.56 (ddt, J=16.9, 10.3, 6.5 Hz, 1H), 1.20 (q, J=11.5 Hz, 1H), 1.05 (d, J=7.0 Hz, 3H), 0.97 (d, J=6.8 Hz, 3H), 0.93 (d, J=6.8 Hz, 3H), 0.87 (d, J=6.9 Hz, 3H), 0.84-0.80 (m, 9H). ¹³C NMR (176 MHz, acetone) δ 171.92, 166.88, 144.96, 144.37, 142.46, 134.02, 133.38, 132.82, 127.42, 125.50, 84.94, 84.12, 83.64, 80.65, 77.80, 77.48, 77.37, 70.56, 70.53, 60.14, 55.85, 55.11, 42.46, 41.47, 41.00, 39.13, 38.86, 38.26, 36.92, 34.40, 29.35, 22.63, 21.56, 20.25, 18.03, 15.03, 14.83, 14.33, 12.75, 10.74, 8.85.

Bafilomycin esterification general procedure.

DMAP, EDC, and carboxylic acid were added to a solution of bafilomycin A₁ in DCM under anhydrous conditions. The reaction was stirred overnight then concentrated under reduced pressure. The crude reaction mix was purified by preparative TLC (pTLC).

### Comparative Example 8: Synthesis of 21-Acetyl Bafilomycin (S9)

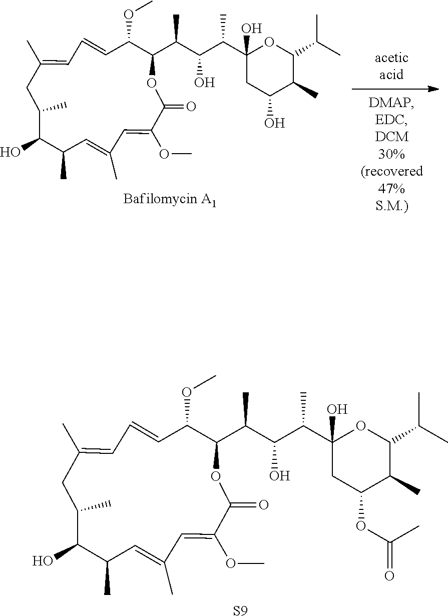

Amounts: DMAP (6.47 mg, 53.0 μmol, 2.1 eq), EDC (13.9 mg, 55.5 μmol, 2.2 eq), acetic acid (1.0 eq, approximately 0.25 mL of a 1.44 μg/mL solution), bafilomycin A₁ (15.0 mg, 25.2 μmol), and DCM (2.92 mL). Purified by pTLC (20% acetone in hexanes) to yield the product. 21-Acetyl Bafilomycin ¹H NMR (599 MHz, Acetone) δ 6.71 (d, J=0.8 Hz, 1H), 6.68 (dd, J=15.0, 10.8 Hz, 1H), 5.96 (dt, J=8.9, 1.2 Hz, 1H), 5.80 (d, J=10.9 Hz, 1H), 5.38 (d, J=2.1 Hz, 1H), 5.14 (dd, J=15.0, 9.2 Hz, 1H), 4.97 (dd, J=8.4, 1.4 Hz, 1H), 4.91 (td, J=10.9, 4.8 Hz, 1H), 4.76 (dd, J=4.4, 1.1 Hz, 1H), 4.18 (ddd, J=10.7, 4.4, 1.9 Hz, 1H), 4.09-4.03 (m, 2H), 3.64 (s, 3H), 3.58 (dd, J=10.4, 2.2 Hz, 1H), 3.31 (td, J=6.5, 5.7, 2.0 Hz, 1H), 3.24 (s, 3H), 2.55 (tt, J=9.2, 6.5 Hz, 1H), 2.25 (dd, J=11.8, 4.9 Hz, 1H), 2.20-2.12 (m, 1H), 2.08-1.99 (m, 2H), 2.00 (s, 3H), 1.98 (d, J=1.2 Hz, 3H), 1.97-1.84 (m, 5H), 1.83 (dt, J=8.8, 6.4 Hz, 1H), 1.52 (tdd, J=13.0, 8.5, 5.2 Hz, 1H), 1.21 (td, J=11.5, 2.1 Hz, 1H), 1.05 (d, J=7.0 Hz, 3H), 0.99 (d, J=7.2 Hz, 3H), 0.94 (d, J=6.8 Hz, 3H), 0.92 (d, J=6.8 Hz, 3H), 0.87 (d, J=6.9 Hz, 3H), 0.81 (d, J=3.7 Hz, 3H), 0.80 (d, J=4.1 Hz, 3H). ¹³C NMR (151 MHz, Acetone) δ 170.62, 167.80, 145.72, 144.83, 141.95, 134.45, 134.20, 132.78, 127.02, 125.23, 99.71, 83.36, 80.41, 77.41, 76.57, 74.14, 71.57, 60.17, 55.67, 42.96, 42.27, 41.70, 40.78, 38.99, 38.29, 38.01, 28.74, 22.25, 21.62, 21.07, 20.35, 17.73, 14.59, 14.15, 12.53, 10.30, 7.36.

### Comparative Example 9: Synthesis of 7,21-Diacetyl Bafilomycin A₁

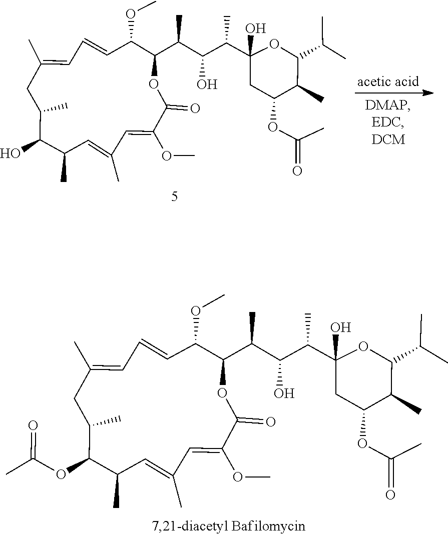

Amounts: DMAP (6.0 mg, 50. μmol, 2.1 eq), EDC (9.9 mg, 52 μmol, 2.2 eq), acetic acid (2.7 μL, 47 μL, 2.0 eq), 21-acetyl bafilomycin A₁ (15.0 mg, 24 μmol), and DCM (2.9 mL). Purified by pTLC (20% acetone in hexanes) to yield the product. Diacetyl Baf ¹H NMR (599 MHz, Acetone) δ 6.72 (dd, J=15.0, 10.6 Hz, 1H), 6.70 (d, J=0.9 Hz, 1H), 5.91 (d, J=10.7 Hz, 1H), 5.81 (dp, J=9.0, 1.2 Hz, 1H), 5.36 (d, J=2.2 Hz, 1H), 5.24 (dd, J=15.1, 9.2 Hz, 1H), 4.99 (dd, J=8.3, 1.5 Hz, 1H), 4.92 (td, J=10.9, 4.8 Hz, 1H), 4.74 (dd, J=6.5, 2.5 Hz, 1H), 4.71 (dd, J=4.4, 1.1 Hz, 1H), 4.19 (ddd, J=10.7, 4.4, 1.9 Hz, 1H), 4.09 (t, J=8.7 Hz, 1H), 3.65 (s, 3H), 3.58 (dd, J=10.4, 2.2 Hz, 1H), 3.25 (s, 3H), 2.80-2.74 (m, 1H), 2.26 (dd, J=11.8, 4.8 Hz, 1H), 2.21-2.15 (m, 1H), 2.15 (s, 3H), 2.04-2.01 (m, 1H), 2.00 (d, J=1.7 Hz, 6H), 1.93 (td, J=6.8, 2.2 Hz, 1H), 1.91 (d, J=1.3 Hz, 3H), 1.86-1.81 (m, 1H), 1.73 (dd, J=14.9, 11.5 Hz, 1H), 1.53 (tq, J=10.4, 6.5 Hz, 1H), 1.21 (td, J=11.5, 2.1 Hz, 1H), 1.04 (d, J=6.9 Hz, 3H), 0.99 (d, J=7.2 Hz, 3H), 0.95 (d, J=6.8 Hz, 3H), 0.92 (d, J=7.0 Hz, 3H), 0.89 (d, J=6.9 Hz, 3H), 0.81 (d, J=2.5 Hz, 3H), 0.80 (d, J=2.8 Hz, 3H). ¹³C NMR (151 MHz, Acetone) δ 171.53, 170.60, 167.58, 143.39, 142.72, 142.01, 133.93, 133.20, 128.29, 126.00, 99.71, 83.35, 81.87, 77.59, 76.59, 74.13, 71.57, 60.16, 55.80, 42.99, 42.05, 40.79, 39.29, 39.00, 38.38, 36.98, 28.74, 22.36, 21.63, 21.25, 21.07, 20.63, 17.16, 14.60, 14.12, 12.54, 10.37, 7.36.

### Comparative Example 10: Synthesis of 21-Pentanoate Bafilomycin A₁ (S10)

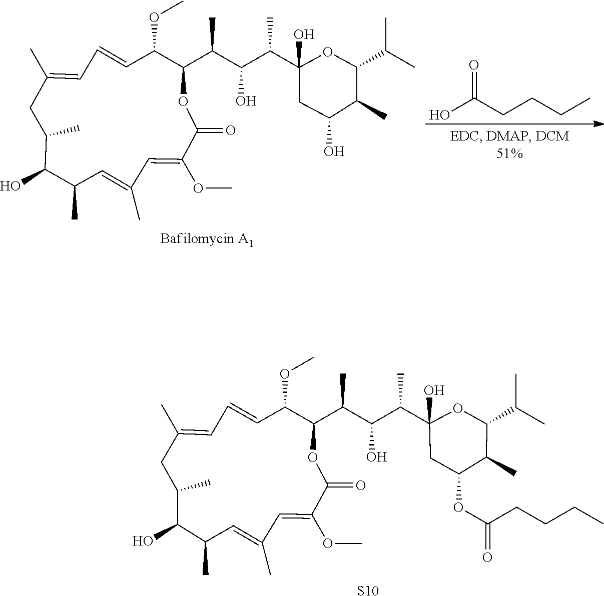

Amounts: DMAP (4.9 mg, 41 μmol, 2.1 eq), EDC (8.1 mg, 42 μmol, 2.2 eq), pentanoic acid (3.9 mg, 39 μmol, 2 eq), bafilomycin A₁ (12 mg, 20 μmol), and DCM (2.5 mL). Purified by pTLC (20% acetone in hexanes) to yield the product. 21-pentanone Bafilomycin ¹H NMR (599 MHz, Acetone) δ 6.71 (s, 1H), 6.68 (dd, J=15.0, 10.8 Hz, 1H), 5.96 (d, J=8.8 Hz, 1H), 5.80 (d, J=10.8 Hz, 1H), 5.38 (d, J=2.1 Hz, 1H), 5.15 (dd, J=15.0, 9.2 Hz, 1H), 4.97 (dd, J=8.4, 1.4 Hz, 1H), 4.93 (td, J=10.8, 4.8 Hz, 1H), 4.76 (dd, J=4.3, 1.0 Hz, 1H), 4.18 (ddd, J=10.7, 4.3, 1.8 Hz, 1H), 4.09-4.03 (m, 2H), 3.64 (s, 3H), 3.59 (dd, J=10.4, 2.2 Hz, 1H), 3.31 (ddd, J=7.2, 5.5, 2.0 Hz, 1H), 3.24 (s, 3H), 2.56 (pd, J=7.0, 3.5 Hz, 1H), 2.30 (td, J=7.5, 4.0 Hz, 2H), 2.25 (dd, J=11.7, 4.8 Hz, 1H), 2.21-2.12 (m, 1H), 2.09-1.98 (m, 2H), 1.98 (d, J=1.2 Hz, 3H), 1.93 (s, 3H), 1.89 (dtd, J=18.0, 7.0, 3.1 Hz, 2H), 1.83 (dd, J=8.1, 6.3 Hz, 1H), 1.63-1.55 (m, 2H), 1.58-1.49 (m, 1H), 1.35 (h, J=7.4 Hz, 2H), 1.21 (td, J=11.5, 2.1 Hz, 1H), 1.05 (d, J=7.0 Hz, 3H), 0.99 (d, J=7.1 Hz, 3H), 0.95 (d, J=6.9 Hz, 3H), 0.92 (d, J=5.0 Hz, 3H), 0.91 (t, J=6.4 Hz, 3H), 0.88 (d, J=6.8 Hz, 3H), 0.81 (d, J=3.7 Hz, 3H), 0.80 (d, J=3.9 Hz, 3H). ¹³C NMR (151 MHz, Acetone) δ 173.28, 167.82, 145.71, 144.82, 141.95, 134.45, 134.20, 132.79, 127.04, 125.24, 99.72, 83.34, 80.42, 77.42, 76.57, 73.97, 71.58, 60.17, 55.67, 42.95, 42.27, 41.70, 40.82, 39.00, 38.28, 38.01, 34.66, 28.75, 27.90, 22.88, 22.25, 21.63, 20.36, 17.73, 14.60, 14.16, 14.01, 12.58, 10.30, 7.38.

### Comparative Example 11: Synthesis of 21-Nonanoate Bafilomycin A₁ (S11)

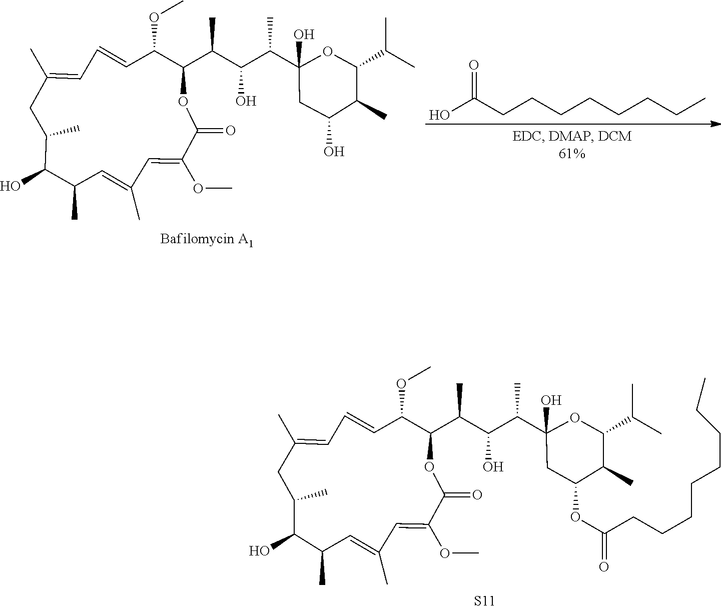

Amounts: DMAP (4.9 mg, 41 μmol, 2.1 eq), EDC (8.1 mg, 42 μmol, 2.2 eq), nonanoic acid (6.10 mg, 38.5 μmol, 2 eq), bafilomycin A₁ (12 mg, 20 μmol), and DCM (2.5 mL). Purified by pTLC (20% acetone in hexanes) to yield the product. 21-nonanoate Bafilomycin ¹H NMR (599 MHz, CDCl₃) δ 6.67 (s, 1H), 6.50 (dd, J=15.0, 10.6 Hz, 1H), 5.81 (d, J=10.6 Hz, 1H), 5.77 (d, J=9.1 Hz, 1H), 5.47 (d, J=2.0 Hz, 1H), 5.16 (dd, J=15.0, 9.3 Hz, 1H), 5.01-4.93 (m, 2H), 4.61 (d, J=4.1 Hz, 1H), 4.15-4.09 (m, 1H), 3.88 (t, J=9.0 Hz, 1H), 3.64 (s, 3H), 3.60 (dd, J=10.4, 2.2 Hz, 1H), 3.30 (dd, J=7.1, 3.9 Hz, 1H), 3.24 (s, 3H), 2.58-2.50 (m, 1H), 2.32 (dd, J=11.8, 4.8 Hz, 1H), 2.28 (t, J=7.5 Hz, 2H), 2.13 (td, J=13.6, 12.3, 5.3 Hz, 2H), 1.99 (s, 3H), 1.94 (s, 4H), 1.93-1.84 (m, 2H), 1.75 (q, J=7.2 Hz, 1H), 1.62 (q, J=7.2, 5.2 Hz, 2H), 1.54 (q, J=4.1 Hz, 1H), 1.34-1.23 (m, 12H), 1.17 (td, J=11.4, 2.1 Hz, 1H), 1.10 (s, OH), 1.07 (d, J=7.0 Hz, 3H), 1.02 (d, J=7.1 Hz, 3H), 0.94 (d, J=6.4 Hz, 3H), 0.91 (d, J=6.8 Hz, 2H), 0.88 (t, J=6.9 Hz, 3H), 0.82 (t, J=6.5 Hz, 6H), 0.77 (d, J=6.7 Hz, 3H). ¹³C NMR (151 MHz, CDCl₃) δ 173.18, 167.28, 142.98, 142.70, 141.29, 133.48, 133.02, 132.98, 127.26, 125.31, 98.81, 82.23, 81.22, 76.85, 75.58, 73.60, 70.61, 59.94, 55.52, 42.06, 41.21, 40.20, 40.01, 38.21, 37.17, 36.69, 34.74, 31.81, 29.25-29.13 (m), 27.92, 25.16, 22.65, 21.66, 21.10, 20.17, 17.28, 14.29, 14.07 (d, J=9.3 Hz), 12.30, 9.84, 7.08.

### Comparative Example 12: Synthesis of 21-penta-3-enoate Bafilomycin A₁ (S29)

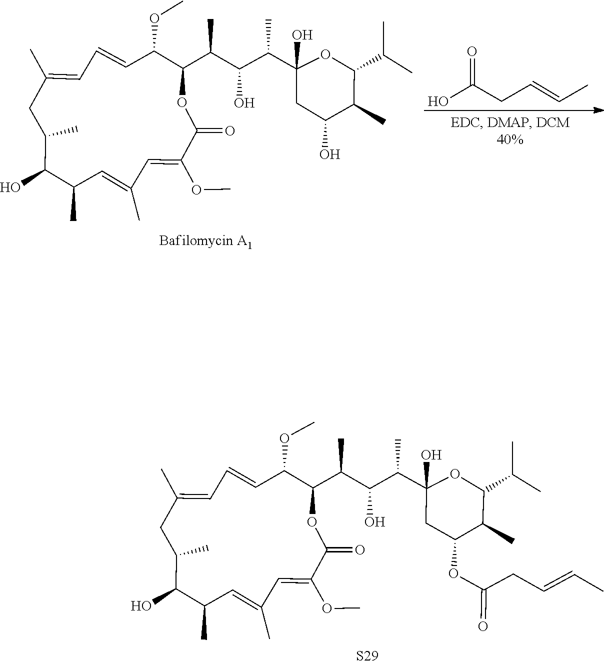

Amounts: DMAP (5.4 mg, 44 μmol, 2.1 eq), EDC (8.8 mg, 46 μmol, 2.2 eq), (E)-pent-3-enoic acid (4.2 mg, 42 μmol, 2 eq), bafilomycin A₁ (13 mg, 21 μmol), and DCM (2.4 mL). Purified by pTLC (20% acetone in hexanes) to yield the product. 21-pent-3-enoate Bafilomycin ¹H NMR (599 MHz, Acetone) δ 6.71 (s, 1H), 6.68 (dd, J=14.9, 10.8 Hz, 1H), 5.96 (d, J=8.8 Hz, 1H), 5.80 (d, J=10.7 Hz, 1H), 5.65-5.51 (m, 2H), 5.38 (d, J=2.1 Hz, 1H), 5.14 (dd, J=15.0, 9.1 Hz, 1H), 4.97 (dd, J=8.4, 1.5 Hz, 1H), 4.92 (td, J=10.9, 4.8 Hz, 1H), 4.76 (dd, J=4.3, 1.2 Hz, 1H), 4.18 (ddd, J=10.8, 4.4, 1.8 Hz, 1H), 4.10-4.03 (m, 2H), 3.64 (d, J=1.2 Hz, 3H), 3.58 (dd, J=10.4, 2.2 Hz, 1H), 3.35-3.28 (m, 1H), 3.24 (d, J=1.2 Hz, 3H), 3.06-2.96 (m, 2H), 2.84 (s, 1H), 2.60-2.52 (m, 1H), 2.25 (dd, J=11.7, 4.8 Hz, 1H), 2.20-2.12 (m, 1H), 2.02 (d, J=11.0 Hz, 2H), 1.98 (d, J=1.4 Hz, 3H), 1.93 (s, 3H), 1.96-1.86 (m, 1H), 1.83 (q, J=7.1 Hz, 1H), 1.68-1.64 (m, 3H), 1.59-1.49 (m, 1H), 1.25-1.18 (m, 1H), 1.05 (d, J=6.9 Hz, 3H), 0.99 (d, J=7.2 Hz, 3H), 0.95 (d, J=6.8 Hz, 3H), 0.92 (d, J=6.7 Hz, 3H), 0.88 (d, J=6.8 Hz, 3H), 0.80 (dd, J=6.7, 1.5 Hz, 6H). ¹³C NMR (151 MHz, Acetone) δ 171.66, 167.81, 145.71, 144.82, 141.95, 134.45, 134.20, 132.79, 129.21, 127.03, 125.24, 124.41, 99.72, 83.34, 80.42, 77.41, 76.55, 74.33, 71.57, 60.17, 55.67, 42.94, 42.27, 41.70, 40.76, 39.02, 38.67, 38.28, 38.01, 28.73, 22.25, 21.62, 20.36, 18.00, 17.73, 14.60, 14.16, 12.53, 10.29, 7.38.

### Comparative Example 13: Synthesis of 21-penta-2,4-dienoate Bafilomycin A₁ (S12)

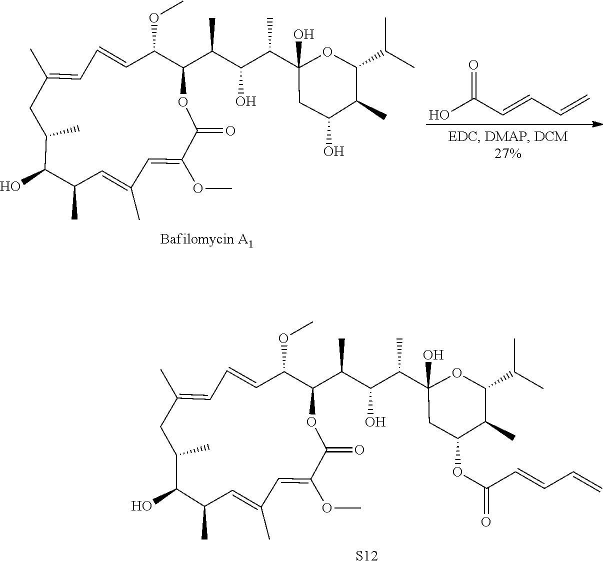

Amounts: DMAP (6.2 mg, 51 μmol, 2.1 eq), EDC (10 mg, 53 μmol, 2.2 eq), (E)-penta-2,4-dienoic acid (4.7 mg, 48 μmol, 2 eq), bafilomycin A₁ (15 mg, 24 μmol), and DCM (2.9 mL). Purified by pTLC (20% acetone in hexanes) to yield the product. 21-penta-2,4-dienoate Bafilomycin ¹H NMR (599 MHz, Acetone) δ 7.27 (dd, J=15.4, 11.0 Hz, 1H), 6.72 (d, J=1.0 Hz, 1H), 6.68 (dd, J=15.0, 10.8 Hz, 1H), 6.58 (dt, J=17.0, 10.5 Hz, 1H), 6.02-5.96 (m, 1H), 5.96 (d, J=9.1 Hz, 1H), 5.80 (d, J=10.8 Hz, 1H), 5.73-5.67 (m, 1H), 5.52 (dd, J=10.2, 1.6 Hz, 1H), 5.41 (t, J=1.8 Hz, 1H), 5.15 (dd, J=15.0, 9.2 Hz, 1H), 5.01 (td, J=10.9, 4.8 Hz, 1H), 4.97 (dd, J=8.3, 1.5 Hz, 1H), 4.77 (dd, J=4.4, 1.1 Hz, 1H), 4.19 (ddd, J=10.8, 4.5, 1.8 Hz, 1H), 4.09-4.03 (m, 2H), 3.64 (d, J=1.3 Hz, 3H), 3.63-3.59 (m, 1H), 3.31 (td, J=6.4, 5.5, 1.7 Hz, 1H), 3.24 (d, J=1.3 Hz, 3H), 2.56 (p, J=7.9, 7.5 Hz, 1H), 2.30 (dd, J=11.8, 4.9 Hz, 1H), 2.21-2.13 (m, 1H), 2.02 (s, 2H), 1.98 (d, J=1.3 Hz, 3H), 1.94 (s, 1H), 1.93 (d, J=1.4 Hz, 3H), 1.85 (q, J=7.3 Hz, 1H), 1.60 (td, J=10.4, 6.4 Hz, 1H), 1.30-1.23 (m, 1H), 1.05 (d, J=6.9 Hz, 3H), 1.00 (d, J=7.1 Hz, 3H), 0.96 (d, J=6.8 Hz, 2H), 0.92 (d, J=6.7 Hz, 3H), 0.88 (d, J=6.8 Hz, 3H), 0.82 (dd, J=6.7, 3.5 Hz, 6H). ¹¹C NMR (151 MHz, Acetone) δ 167.82, 166.52, 145.71, 145.34, 144.82, 141.95, 135.91, 134.45, 134.21, 132.79, 127.04, 125.95, 125.24, 123.44, 99.75, 83.35, 80.42, 77.42, 76.56, 74.38, 71.59, 60.17, 55.67, 42.98, 42.27, 41.70, 40.82, 39.09, 38.29, 38.01, 28.76, 22.25, 21.63, 20.36, 17.73, 14.61, 14.16, 12.58, 10.30, 7.37.

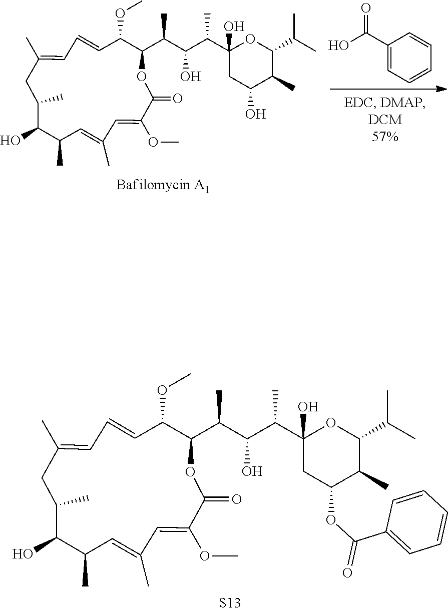

21-benzoate Bafilomycin A₁ (S13). Amounts: DMAP (4.9 mg, 41 μmol, 2.1 eq), EDC (8.1 mg, 42 μmol, 2.2 eq), benzoic acid (4.71 mg, 38.5 μmol, 2 eq), bafilomycin A₁ (12 mg, 20 μmol), and DCM (2.4 mL). Purified by pTLC (20% acetone in hexanes) to yield 8.0 mg of a white solid (57%). 21-benzyl Bafilomycin ¹H NMR (599 MHz, Acetone) δ 8.07-8.02 (m, 2H), 7.66-7.61 (m, 1H), 7.56-7.49 (m, 2H), 6.72 (d, J=0.9 Hz, 1H), 6.68 (dd, J=15.0, 10.8 Hz, 1H), 5.97 (dt, J=8.9, 1.2 Hz, 1H), 5.81 (d, J=10.8 Hz, 1H), 5.45 (d, J=2.0 Hz, 1H), 5.20 (dt, J=10.8, 5.4 Hz, 1H), 5.15 (dd, J=14.8, 9.0 Hz, 1H), 4.98 (dd, J=8.4, 1.4 Hz, 1H), 4.79 (dd, J=4.3, 1.0 Hz, 1H), 4.21 (ddd, J=10.7, 4.4, 1.8 Hz, 1H), 4.10-4.03 (m, 2H), 3.68 (dd, J=10.4, 2.3 Hz, 1H), 3.65 (s, 3H), 3.31 (td, J=6.8, 6.1, 2.1 Hz, 1H), 3.24 (s, 3H), 2.56 (tt, J=9.0, 7.0 Hz, 1H), 2.42 (dd, J=11.8, 4.9 Hz, 1H), 2.22-2.13 (m, 1H), 2.03 (d, J=11.0 Hz, 2H), 1.99 (d, J=1.2 Hz, 3H), 1.99-1.95 (m, 1H), 1.93 (d, J=1.2 Hz, 3H), 1.88 (qd, J=7.5, 2.7 Hz, 2H), 1.76 (ddt, J=16.9, 10.4, 6.5 Hz, 1H), 1.39 (td, J=11.5, 2.0 Hz, 1H), 1.06 (d, J=7.0 Hz, 3H), 1.02 (d, J=7.1 Hz, 3H), 0.98 (d, J=6.8 Hz, 3H), 0.92 (d, J=6.8 Hz, 3H), 0.89 (dd, J=6.7, 1.9 Hz, 6H), 0.85 (d, J=6.8 Hz, 3H). ¹³C NMR (151 MHz, Acetone) δ 167.8, 166.4, 145.7, 144.8, 142.0, 134.5, 134.2, 133.8, 132.8, 131.7, 130.2, 129.4, 127.1, 125.3, 99.8, 83.4, 80.4, 77.4, 76.6, 75.3, 71.6, 60.2, 55.7, 43.0, 42.3, 41.7, 40.8, 39.2, 38.3, 38.0, 28.8, 22.3, 21.7, 20.4, 17.7, 14.7, 14.2, 12.7, 10.3, 7.4. LC-MS (IT): calcd for C₄₂H₆₂O₁₀ [M+CH₃OH+H]⁺ 759.6. found, 760.5.

### Comparative Example 14: Synthesis of 21-penta-4-ene Bafilomycin

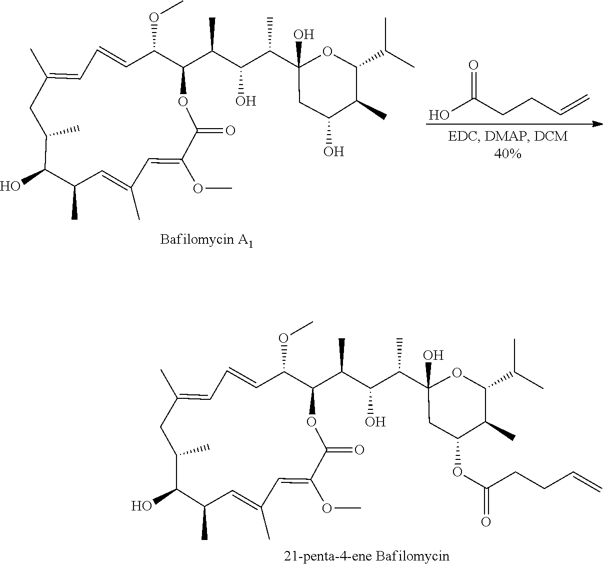

21-pent-4-ene Baf 1H NMR (599 MHz, Acetone) δ 6.74 (s, 1H), 6.71 (dd, J=15.0, 10.9 Hz, 1H), 5.99 (d, J=8.8 Hz, 1H), 5.93-5.81 (m, 2H), 5.41 (d, J=2.2 Hz, 1H), 5.17 (dd, J=15.1, 9.3 Hz, 1H), 5.09 (dt, J=17.1, 1.7 Hz, 1H), 5.02-4.94 (m, 3H), 4.79 (d, J=4.3 Hz, 1H), 4.21 (dd, J=11.1, 4.1 Hz, 1H), 4.12-4.06 (m, 2H), 3.67 (d, J=1.4 Hz, 3H), 3.61 (d, J=10.6 Hz, 1H), 3.36-3.31 (m, 1H), 3.27 (d, J=1.4 Hz, 3H), 2.81 (d, J=1.4 Hz, 1H), 2.62-2.53 (m, 1H), 2.43 (dd, J=8.0, 5.3 Hz, 2H), 2.40-2.32 (m, 3H), 2.28 (dd, J=11.7, 4.6 Hz, 1H), 2.19 (dq, J=13.6, 7.1 Hz, 1H), 2.04 (s, 1H), 2.01 (d, J=1.4 Hz, 3H), 1.96 (s, 4H), 1.86 (t, J=7.3 Hz, 1H), 1.57 (h, J=8.9, 8.2 Hz, 1H), 1.24 (t, J=11.4 Hz, 1H), 1.08 (d, J=7.2 Hz, 3H), 1.01 (dd, J=7.1, 1.4 Hz, 3H), 0.98 (d, J=7.0 Hz, 2H), 0.95 (d, J=6.8 Hz, 2H), 0.93-0.88 (m, 4H), 0.86-0.81 (m, 7H). 13C NMR (151 MHz, Acetone) δ 172.63, 167.82, 145.72, 144.83, 141.96, 138.03, 134.46, 134.21, 132.79, 127.04, 125.25, 115.64, 99.72, 83.35, 80.43, 77.42, 76.58, 74.21, 71.58, 60.17, 55.67, 42.96, 42.28, 41.71, 40.84, 38.98, 38.28, 38.01, 34.27, 28.75, 22.25, 21.63, 20.36, 17.73, 14.60, 14.16, 12.60, 10.30, 7.38.

### Comparative Example 15: Synthesis of 21-(3-(3-(but-3-yn-1-yl)-3H-diazirin-3-yl)propanoate) Bafilomycin

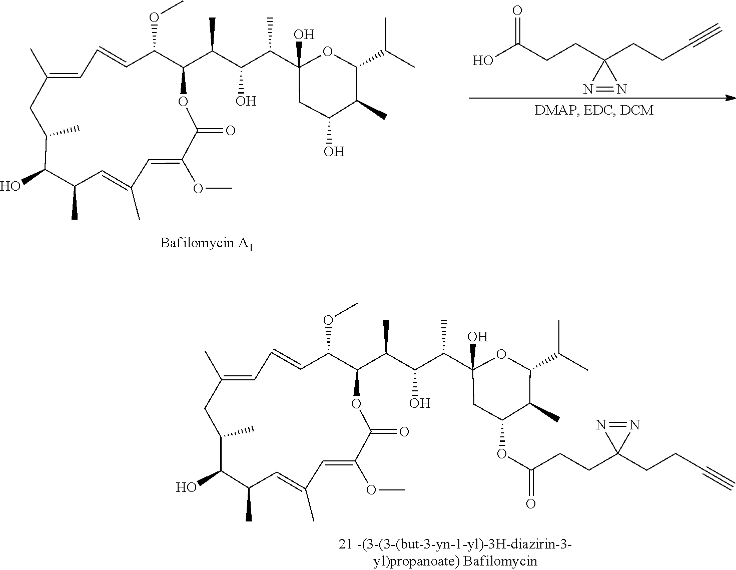 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(O)C[C@@H](O)[C@H](C)[C@@H](C(C)C)O2)OC1=O ; C#CCCC1(CCC(=O)O)N=N1 ; C#CCCC1(CCC(=O)O[C@@H]2C[C@](O)([C@@H](C)[C@H](O)[C@H](C)[C@H]3OC(=O)/C(OC)=C/C(C)=C/[C@@H](C)[C@@H](O)[C@@H](C)C/C(C)=C/C=C/[C@@H]3OC)O[C@H](C(C)C)[C@H]2C)N=N1]

¹H NMR (599 MHz, Acetone) δ 6.71 (d, J=0.9 Hz, 1H), 6.68 (dd, J=15.0, 10.8 Hz, 1H), 5.96 (dt, J=8.8, 1.2 Hz, 1H), 5.83-5.78 (m, 1H), 5.39 (d, J=2.1 Hz, 1H), 5.14 (dd, J=15.0, 9.2 Hz, 1H), 4.99-4.94 (m, 1H), 4.97-4.91 (m, 1H), 4.76 (dd, J=4.4, 1.1 Hz, 1H), 4.18 (ddd, J=10.7, 4.4, 1.9 Hz, 1H), 4.09-4.02 (m, 2H), 3.64 (s, 3H), 3.58 (dd, J=10.4, 2.2 Hz, 1H), 3.31 (ddd, J=7.3, 5.5, 2.0 Hz, 1H), 3.24 (s, 3H), 2.60-2.51 (m, 1H), 2.39 (t, J=2.7 Hz, 1H), 2.28 (dd, J=11.8, 4.8 Hz, 1H), 2.18 (td, J=7.5, 3.5 Hz, 2H), 2.15 (ddt, J=6.7, 4.3, 2.0 Hz, 1H), 2.09-2.05 (m, 2H), 2.05-2.00 (m, 2H), 1.98 (d, J=1.2 Hz, 3H), 1.96-1.85 (m, 4H), 1.85-1.81 (m, 1H), 1.81-1.77 (m, 2H), 1.66 (t, J=7.5 Hz, 2H), 1.54 (tdd, J=13.1, 8.6, 5.3 Hz, 1H), 1.23 (td, J=11.5, 2.1 Hz, 1H), 1.05 (d, J=7.0 Hz, 3H), 0.99 (d, J=7.2 Hz, 3H), 0.95 (d, J=6.8 Hz, 3H), 0.92 (d, J=6.8 Hz, 3H), 0.87 (dd, J=7.0, 2.8 Hz, 3H), 0.82 (d, J=6.5 Hz, 3H), 0.80 (d, J=6.8 Hz, 3H). ¹³C NMR (151 MHz, Acetone) δ 172.27, 167.95, 145.85, 144.96, 142.08, 134.59, 134.34, 132.92, 127.17, 125.37, 99.86, 83.66, 83.47, 80.56, 77.55, 76.70, 74.75, 71.71, 70.73, 60.30, 55.80, 43.08, 42.41, 41.84, 40.87, 39.11, 38.40, 38.14, 33.10, 29.22, 28.87, 28.83, 28.70, 22.39, 21.76, 20.50, 17.86, 14.73, 14.30, 13.72, 12.72, 10.43, 7.53.

### Comparative Example 16: Synthesis of 21-Pent-4-ynoate-Bafilomycin A1

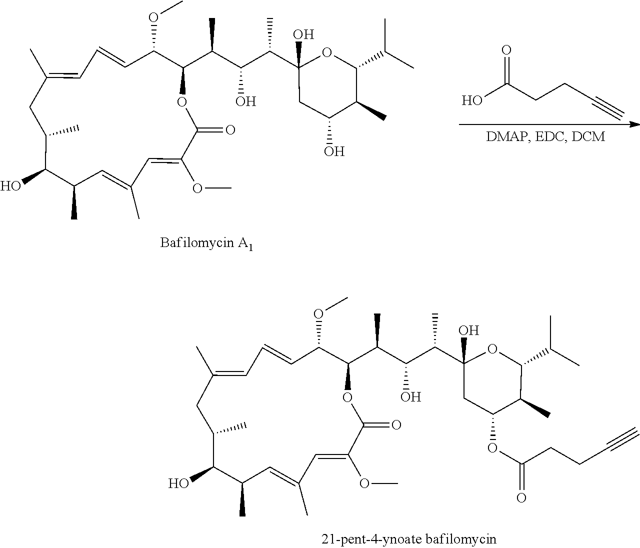

¹H NMR (599 MHz, Acetone) δ 6.71 (s, 1H), 6.68 (dd, J=15.0, 10.8 Hz, 1H), 5.96 (d, J=8.9 Hz, 1H), 5.80 (d, J=10.9 Hz, 1H), 5.39 (d, J=2.1 Hz, 1H), 5.14 (dd, J=15.0, 9.2 Hz, 1H), 5.01-4.93 (m, 2H), 4.76 (dd, J=4.4, 1.1 Hz, 1H), 4.18 (ddd, J=10.7, 4.4, 1.8 Hz, 1H), 4.09-4.03 (m, 2H), 3.64 (d, J=0.7 Hz, 3H), 3.59 (dd, J=10.3, 2.3 Hz, 1H), 3.31 (td, J=6.3, 5.4, 1.8 Hz, 1H), 3.24 (d, J=0.7 Hz, 3H), 2.59-2.51 (m, 3H), 2.51-2.44 (m, 2H), 2.37 (t, J=2.6 Hz, 1H), 2.27 (dd, J=11.8, 4.8 Hz, 1H), 2.16 (dd, J=10.5, 6.9 Hz, 1H), 2.09-2.00 (m, 2H), 1.98 (d, J=1.2 Hz, 3H), 1.93 (s, 3H), 1.93-1.86 (m, 2H), 1.84 (q, J=7.7, 7.1 Hz, 1H), 1.55 (tdd, J=12.9, 8.4, 5.2 Hz, 1H), 1.24 (td, J=11.5, 2.1 Hz, 1H), 1.05 (d, J=7.0 Hz, 3H), 0.99 (d, J=7.1 Hz, 3H), 0.95 (d, J=6.9 Hz, 3H), 0.92 (d, J=6.8 Hz, 3H), 0.88 (d, J=6.8 Hz, 3H), 0.83 (d, J=6.5 Hz, 3H), 0.80 (d, J=6.8 Hz, 3H). ¹³C NMR (151 MHz, Acetone) δ 171.65, 167.82, 145.72, 144.83, 141.95, 134.46, 134.21, 132.79, 127.03, 125.24, 99.73, 83.51, 83.35, 80.42, 77.41, 76.58, 74.60, 71.58, 70.40, 60.17, 55.67, 42.95, 42.27, 41.71, 40.79, 38.98, 38.29, 38.01, 34.26, 28.74, 22.25, 21.63, 20.36, 17.73, 14.88, 14.59, 14.16, 12.59, 10.30, 7.38.

### Comparative Example 17: Synthesis of 21-pent-4-ynoate-7-(3-(trifluoromethyl)-3H-diazirin-3-yl)benzoate Bafilomycin

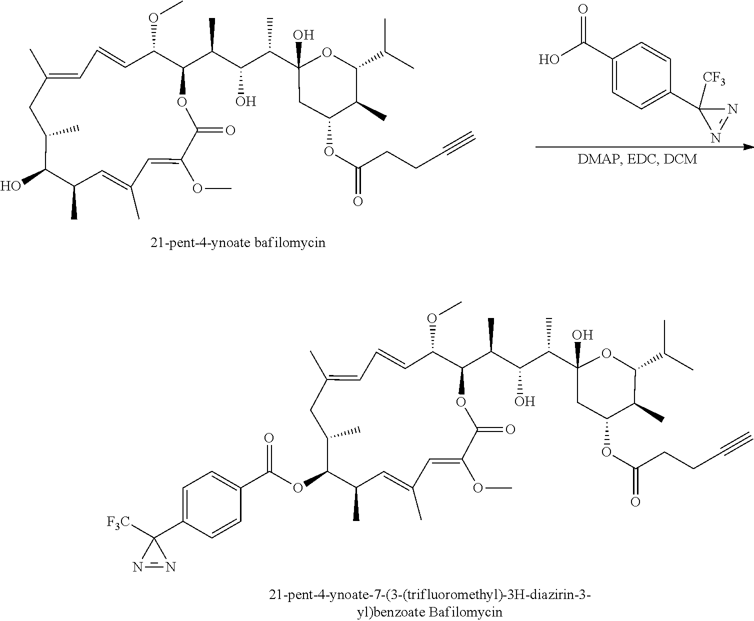 [SMILES: C#CCCC(=O)O[C@@H]1C[C@](O)([C@@H](C)[C@H](O)[C@H](C)[C@H]2OC(=O)/C(OC)=C/C(C)=C/[C@@H](C)[C@@H](O)[C@@H](C)C/C(C)=C/C=C/[C@@H]2OC)O[C@H](C(C)C)[C@H]1C ; O=C(O)c1ccc(C2(C(F)(F)F)N=N2)cc1 ; C#CCCC(=O)O[C@@H]1C[C@](O)([C@@H](C)[C@H](O)[C@H](C)[C@H]2OC(=O)/C(OC)=C/C(C)=C/[C@@H](C)[C@@H](OC(=O)c3ccc(C4(C(F)(F)F)N=N4)cc3)[C@@H](C)C/C(C)=C/C=C/[C@@H]2OC)O[C@H](C(C)C)[C@H]1C]

¹H NMR (599 MHz, Acetone) δ 8.21-8.16 (m, 2H), 7.47 (d, J=8.2 Hz, 2H), 6.79 (s, 1H), 6.69 (dd, J=15.1, 10.6 Hz, 1H), 6.05 (d, J=9.0 Hz, 1H), 5.96 (d, J=10.6 Hz, 1H), 5.38 (d, J=2.0 Hz, 1H), 5.28 (dd, J=15.0, 9.1 Hz, 1H), 5.02 (dd, J=8.1, 1.4 Hz, 1H), 4.98 (td, J=11.0, 4.9 Hz, 1H), 4.95 (dd, J=6.7, 2.3 Hz, 1H), 4.72 (d, J=4.4 Hz, 1H), 4.22 (ddd, J=10.7, 4.5, 1.8 Hz, 1H), 4.09 (t, J=8.6 Hz, 1H), 3.68 (s, 3H), 3.60 (dd, J=10.3, 2.2 Hz, 1H), 3.31 (d, J=3.5 Hz, 1H), 3.22 (s, 3H), 3.12-3.08 (m, 1H), 2.94 (tt, J=9.2, 7.0 Hz, 1H), 2.78 (s, 1H), 2.58-2.52 (m, 2H), 2.51-2.45 (m, 2H), 2.38 (t, J=2.6 Hz, 1H), 2.28 (dd, J=11.8, 4.8 Hz, 1H), 2.25-2.13 (m, 3H), 2.06 (s, 3H), 1.98-1.90 (m, 1H), 1.90-1.83 (m, 1H), 1.77 (dd, J=15.3, 11.5 Hz, 1H), 1.62-1.53 (m, 1H), 1.52 (s, 3H), 1.25 (td, J=11.5, 2.0 Hz, 1H), 1.14 (d, J=6.8 Hz, 3H), 1.00 (dd, J=8.8, 7.0 Hz, 6H), 0.96 (d, J=6.8 Hz, 3H), 0.93 (d, J=6.8 Hz, 3H), 0.83 (dd, J=9.2, 6.6 Hz, 6H). ¹³C NMR (151 MHz, Acetone) δ 171.65, 167.59, 166.53, 142.98, 142.79, 141.52, 134.33, 134.17, 133.59, 133.17, 132.63, 131.24, 128.72, 127.73, 126.08, 123.85, 122.03, 99.74, 84.12, 83.52, 83.20, 77.80, 76.60, 74.59, 71.59, 70.41, 60.19, 55.76, 42.99, 41.82, 40.79, 39.11, 38.99, 38.45, 37.13, 34.27, 28.75, 22.03, 21.64, 20.67, 17.48, 14.89, 14.61, 14.24, 12.60, 10.43, 7.36.

### Comparative Example 18: Synthesis of 21-(3-(trifluoromethyl)-3H-diazirin-3-yl)benzoate Bafilomycin

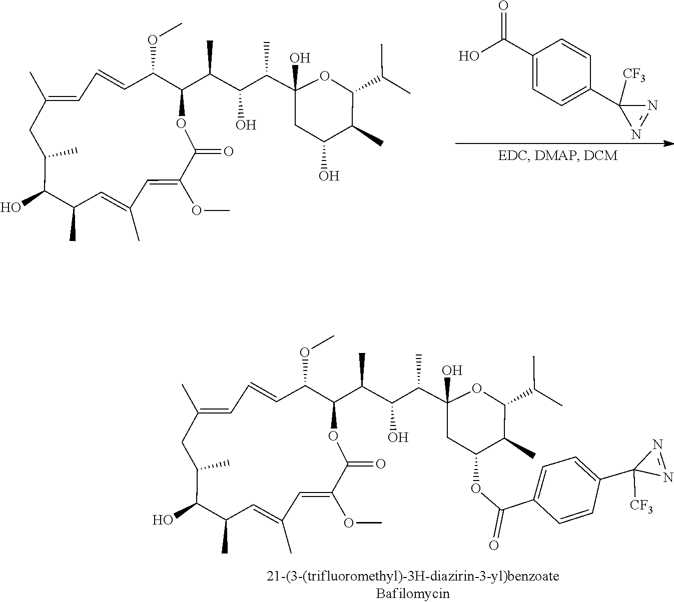 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(O)C[C@@H](O)[C@H](C)[C@@H](C(C)C)O2)OC1=O ; O=C(O)c1ccc(C2(C(F)(F)F)N=N2)cc1 ; CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(O)C[C@@H](OC(=O)c3ccc(C4(C(F)(F)F)N=N4)cc3)[C@H](C)[C@@H](C(C)C)O2)OC1=O]

¹H NMR (599 MHz, Acetone) δ 8.18-8.13 (m, 2H), 7.44 (d, J=8.2 Hz, 2H), 6.72 (d, J=0.9 Hz, 1H), 6.68 (dd, J=15.0, 10.8 Hz, 1H), 5.97 (dt, J=8.9, 1.2 Hz, 1H), 5.81 (d, J=10.8 Hz, 1H), 5.47 (d, J=2.0 Hz, 1H), 5.20 (td, J=10.9, 4.8 Hz, 1H), 5.15 (dd, J=15.0, 9.2 Hz, 1H), 4.98 (dd, J=8.4, 1.4 Hz, 1H), 4.79 (dd, J=4.4, 1.0 Hz, 1H), 4.21 (ddd, J=10.7, 4.4, 1.9 Hz, 1H), 4.09-4.03 (m, 2H), 3.70-3.62 (m, 1H), 3.65 (s, 3H), 3.34-3.28 (m, 1H), 3.24 (s, 3H), 2.56 (tt, J=7.2, 5.1 Hz, 1H), 2.41 (dd, J=11.8, 4.9 Hz, 1H), 2.22-2.14 (m, 1H), 2.03 (d, J=11.0 Hz, 2H), 1.99 (d, J=1.2 Hz, 3H), 1.97 (td, J=6.7, 2.1 Hz, 1H), 1.93 (d, J=1.2 Hz, 3H), 1.92-1.85 (m, 2H), 1.81-1.71 (m, 1H), 1.40 (td, J=11.5, 2.1 Hz, 1H), 1.06 (d, J=7.0 Hz, 3H), 1.01 (d, J=7.2 Hz, 3H), 0.98 (d, J=6.9 Hz, 3H), 0.92 (d, J=6.8 Hz, 3H), 0.89 (d, J=4.2 Hz, 3H), 0.88 (d, J=3.8 Hz, 3H), 0.85 (d, J=6.8 Hz, 3H). ¹³C NMR (151 MHz, Acetone) δ 167.97, 165.54, 145.87, 144.97, 142.09, 134.60, 134.37, 133.95, 133.23, 132.93, 131.04, 127.70, 127.17, 125.38, 124.00, 122.18, 99.97, 83.48, 80.56, 77.56, 76.69, 76.12, 71.75, 60.32, 55.81, 49.93, 43.11, 42.41, 41.85, 40.85, 39.24, 38.42, 38.15, 28.92, 22.39, 21.77, 20.50, 17.87, 14.77, 14.30, 12.81, 10.44, 7.52.

### Comparative Example 19: Synthesis of 21-(3-(trifluoromethyl)-3H-diazirin-3-yl)benzoate-7-pent-4-ynoate-Bafilomycin

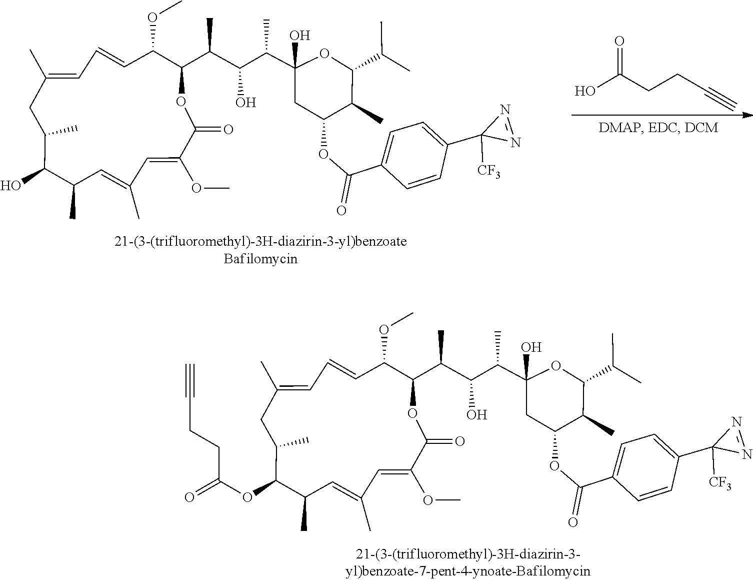

¹H NMR (599 MHz, Acetone) δ 8.18 (dq, J=8.5, 1.9 Hz, 2H), 7.50-7.45 (m, 2H), 6.81-6.77 (m, 1H), 6.68 (dd, J=15.1, 10.6 Hz, 1H), 6.05 (dt, J=9.0, 1.3 Hz, 1H), 5.96 (d, J=10.5 Hz, 1H), 5.38 (d, J=2.1 Hz, 1H), 5.28 (dd, J=15.0, 9.0 Hz, 1H), 5.02 (dt, J=8.3, 1.6 Hz, 1H), 4.98 (td, J=11.0, 4.9 Hz, 1H), 4.95 (dd, J=6.8, 2.2 Hz, 1H), 4.74-4.70 (m, 1H), 4.25-4.19 (m, 1H), 4.09 (t, J=8.7 Hz, 1H), 3.68 (d, J=1.6 Hz, 3H), 3.60 (dd, J=10.4, 2.2 Hz, 1H), 3.22 (s, 3H), 2.94 (ddd, J=9.1, 7.0, 2.2 Hz, 1H), 2.58-2.51 (m, 2H), 2.52-2.45 (m, 2H), 2.38 (t, J=2.6 Hz, 1H), 2.28 (ddd, J=11.8, 4.9, 1.7 Hz, 1H), 2.26-2.13 (m, 3H), 2.04 (s, 3H), 1.98-1.89 (m, 1H), 1.86 (q, J=6.9 Hz, 1H), 1.77 (dd, J=15.4, 11.6 Hz, 1H), 1.62-1.53 (m, 1H), 1.52 (d, J=1.4 Hz, 3H), 1.25 (td, J=11.5, 2.0 Hz, 1H), 1.14 (d, J=6.8 Hz, 3H), 1.02-0.98 (m, 3H), 1.01-0.96 (m, 3H), 0.96 (dd, J=6.9, 1.6 Hz, 3H), 0.93 (d, J=6.9 Hz, 3H), 0.86-0.82 (m, 3H), 0.82 (d, J=6.8 Hz, 3H). ¹³C NMR (151 MHz, Acetone) δ 171.66, 167.59, 166.53, 142.98, 142.79, 141.53, 134.33, 134.17, 133.58, 133.17, 132.63, 131.24, 128.71, 127.73, 126.07, 123.85, 122.31, 99.74, 84.12, 83.52, 83.20, 77.80, 76.61, 74.59, 71.59, 70.41, 60.19, 55.76, 42.99, 41.82, 40.79, 39.11, 38.99, 38.45, 37.13, 34.27, 28.75, 22.03, 21.64, 20.67, 17.47, 14.89, 14.61, 14.23, 12.60, 10.43, 7.36.

### Comparative Example 20: Synthesis of 21-Methoxy CMC

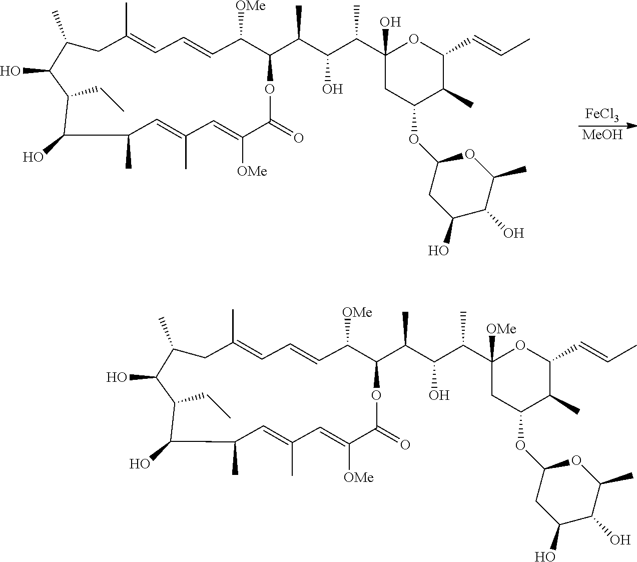 [SMILES: "MeOH" ; C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](CC)[C@@H](O)[C@H](C)C/C(C)=C/C=C/[C@@H]2OC)C[C@@H](O[C@@H]2C[C@H](O)[C@@H](O)[C@H](C)O2)[C@@H]1C ; "FeCl3" ; C/C=C/[C@H]1O[C@@](OC)([C@@H](C)[C@H](O)[C@H](C)[C@H]2OC(=O)/C(OC)=C/C(C)=C/[C@@H](C)[C@@H](O)[C@@H](CC)[C@@H](O)[C@H](C)C/C(C)=C/C=C/[C@@H]2OC)C[C@@H](O[C@@H]2C[C@H](O)[C@@H](O)[C@H](C)O2)[C@@H]1C]

CMC (55 mg, 67 μmol) was dissolved in methanol (5.5 mL) under anhydrous conditions. The reaction flask was cooled to 0° C. before the addition of Iron (Ill) chloride (0.1 mL from a 40 mg/mL solution in methanol, 2.17 mg, 13.4 μmol, 0.2 eq). The reaction was left to stir for 1-3 hours, checking regularly by TLC (12% IPA/CHCl₃). The mixture was quenched with NaHCO₃ then slightly concentrated before it was extracted with ethyl acetate (×3), washed with brine, dried over MgSO₄, filtered and concentrated. The crude material was purified by flash column chromatography using the standard IPA/Hexane/Chloroform conditions (12% IPA with 22% Hexane and 66% CHCl3) yielding the product. Methyl CMC ¹H NMR (599 MHz, CDCl₃) δ 6.52 (dd, J=15.1, 10.7 Hz, 1H), 6.47 (s, 1H), 5.80 (d, J=10.6 Hz, 1H), 5.72-5.64 (m, 1H), 5.42 (ddd, J=15.2, 8.3, 1.7 Hz, 1H), 5.28-5.22 (m, 1H), 5.14 (s, 1H), 4.59 (dd, J=9.6, 1.9 Hz, 1H), 3.82 (t, J=8.9 Hz, 1H), 3.67 (s, 3H), 3.70-3.59 (m, 2H), 3.51 (dd, J=10.3, 8.3 Hz, 1H), 3.44 (dd, J=10.2, 5.0 Hz, 1H), 3.29 (dq, J=9.1, 6.1 Hz, 1H), 3.25 (s, 3H), 3.10 (td, J=9.0, 3.2 Hz, 1H), 3.04 (s, 3H), 2.75 (s, 1H), 2.33 (dd, J=13.6, 5.0 Hz, 1H), 2.28 (s, 1H), 2.23 (d, J=3.7 Hz, 1H), 2.17 (s, 2H), 2.19-2.12 (m, 2H), 2.06 (dd, J=15.1, 7.7 Hz, 2H), 2.02-1.99 (m, 4H), 1.96 (s, 1H), 1.85 (s, 4H), 1.72 (dd, J=6.5, 1.6 Hz, 3H), 1.64-1.57 (m, 1H), 1.53 (dd, J=13.5, 11.1 Hz, 1H), 1.35-1.27 (m, 4H), 1.27-1.22 (m, 3H), 1.21 (s, 2H), 1.07 (s, 5H), 1.00 (d, J=7.0 Hz, 3H), 0.87 (dd, J=9.6, 6.7 Hz, 6H).

### Comparative Example 21: Synthesis of 21-Deoxy CMC (S62)

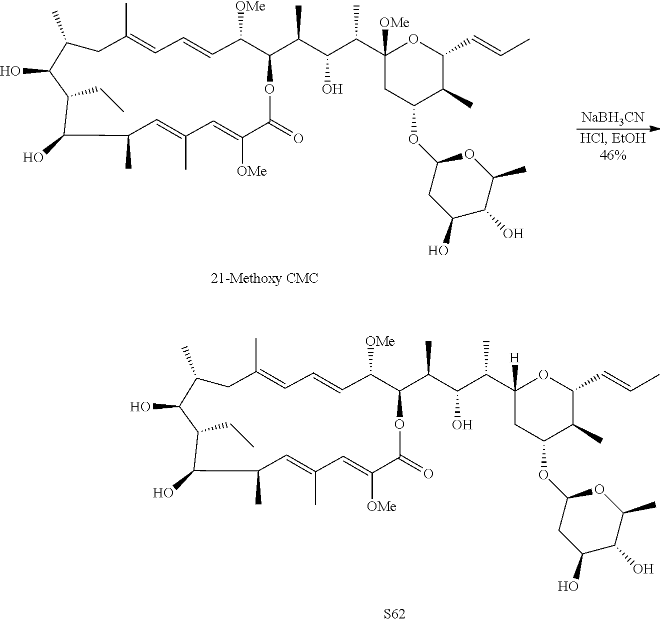 [SMILES: C/C=C/[C@H]1O[C@@](OC)([C@@H](C)[C@H](O)[C@H](C)[C@H]2OC(=O)/C(OC)=C/C(C)=C/[C@@H](C)[C@@H](O)[C@@H](CC)[C@@H](O)[C@H](C)C/C(C)=C/C=C/[C@@H]2OC)C[C@@H](O[C@@H]2C[C@H](O)[C@@H](O)[C@H](C)O2)[C@@H]1C ; "NaBH3CN" ; "HCl, EtOH46%" ; C/C=C/[C@H]1O[C@@H]([C@@H](C)[C@H](O)[C@H](C)[C@H]2OC(=O)/C(OC)=C/C(C)=C/[C@@H](C)[C@@H](O)[C@@H](CC)[C@@H](O)[C@H](C)C/C(C)=C/C=C/[C@@H]2OC)C[C@@H](O[C@@H]2C[C@H](O)[C@@H](O)[C@H](C)O2)[C@@H]1C ; "S62"]

21-methoxy CMC (45 mg, 54 μmol) was dissolved in EtOH (4.68 mL). NaBH₃CN (20 mg, 323 μmol, 6 eq) was added to the solution, followed by addition of 0.5 M HCl (0.47 mL, 4.4 eq). The reaction was allowed to run for 4 hours before neutralized with phosphate buffer. The reaction mixture was then extracted with chloroform twice, washed with water, brine, dried over sodium sulfate, filtered and concentrated. The crude reaction mixture was purified first by flash column chromatography (0-100% IPA in hexane/CHCl₃ mixture (25% hexane in CHCl₃)). The spot corresponding to 21-deoxy CMC was further purified by pTLC (8% IPA/CHCl₃) to yield the product. ¹H NMR (599 MHz, 278 K, CDCl₃) δ 6.57-6.50 (m, 1H), 6.40 (s, 1H), 5.78 (d, J=10.7 Hz, 1H), 5.67 (d, J=9.9 Hz, 1H), 5.57 (dq, J=12.9, 6.2 Hz, 1H), 5.34 (dd, J=15.4, 7.6 Hz, 1H), 5.25 (dd, J=15.1, 8.9 Hz, 1H), 5.19 (d, J=8.7 Hz, 1H), 4.64-4.59 (m, 1H), 3.87-3.77 (m, 3H), 3.68-3.56 (m, 2H), 3.59 (s, 3H), 3.49 (td, J=10.5, 4.5 Hz, 1H), 3.45 (s, 1H), 3.32 (dd, J=10.4, 7.2 Hz, 1H), 3.29-3.26 (m, 1H), 3.26 (s, 3H), 3.25-3.19 (m, 1H), 3.11 (t, J=8.9 Hz, 1H), 2.73 (s, 1H), 2.57 (s, 2H), 2.29 (s, 1H), 2.21-2.05 (m, 3H), 2.03 (s, 1H), 1.97 (s, 3H), 1.95 (s, 2H), 1.86 (s, 3H), 1.72 (dd, J=16.2, 7.4 Hz, 1H), 1.68-1.59 (m, 4H), 1.56-1.45 (m, 1H), 1.33 (d, J=6.0 Hz, 3H), 1.32-1.23 (m, 3H), 1.24-1.15 (m, 3H), 1.10-1.07 (m, 3H), 1.05 (d, J=6.9 Hz, 3H), 0.96 (s, 1H), 0.90 (d, J=7.1 Hz, 3H), 0.87 (d, J=6.5 Hz, 5H), 0.83 (d, J=8.0 Hz, 3H). ¹³C NMR (151 MHz, 278 K, CDCl₃) δ 165.97, 142.10, 141.65, 139.13, 132.81, 132.20, 130.57, 130.27, 128.50, 127.40, 123.10, 95.74, 82.99, 82.03, 79.60, 78.38, 77.63, 76.81, 76.07, 74.51, 71.82, 71.41, 69.56, 59.29, 55.78, 44.61, 43.40, 41.26, 39.37, 39.23, 37.47, 36.46, 34.80, 34.55, 22.86, 21.50, 17.84, 17.67, 16.86, 16.47, 14.19, 13.57, 11.66, 9.62, 8.24.

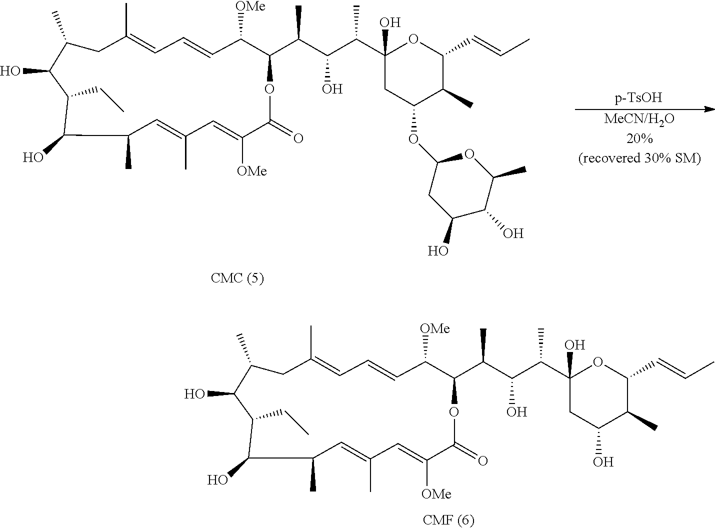

Concanamycin F (6). Concanamycin C (CMC; 60.0 mg, 72.9 mol) was dissolved in MeCN (7.61 mL) and water (1.78 mL) before the addition of pTsOH (55.5, 292 mol, 4 eq). The reaction was ran overnight (between 15-20 hrs) before being cooled to 0° C. and quenched with a saturated solution of sodium bicarbonate. The crude product was extracted with chloroform (3 times), washed with water, dried over Na2SO4, filtered and concentrated. The crude material was purified by FCC using 4-12% isopropanol in a 25% hexanes/chloroform solution. To fully wash the column, it was flushed with 100% isopropanol. The material collected was further purified by pTLC with 4% isopropanol/chloroform to yield 10 mg (19.8%) of 6 as a white solid. Starting material (30 mg, 30%) was also recovered. Concanamycin F ¹H NMR (599 MHz, 278 K, CDCl₃) δ 6.56 (dd, J=15.1, 10.7 Hz, 1H), 6.40 (s, 1H), 5.85-5.82 (m, 1H), 5.78 (d, J=10.5 Hz, 1H), 5.68 (d, J=9.6 Hz, 1H), 5.55 (dt, J=13.3, 6.8 Hz, 1H), 5.32-5.25 (m, 1H), 5.22 (dd, J=15.2, 9.0 Hz, 1H), 5.02 (d, J=9.0 Hz, 1H), 4.67 (d, J=4.3 Hz, 1H), 4.02 (d, J=11.6 Hz, 1H), 3.97 (t, J=9.2 Hz, 1H), 3.86 (t, J=9.1 Hz, 1H), 3.85-3.80 (m, 1H), 3.73 (td, J=10.6, 4.5 Hz, 1H), 3.56 (s, 3H), 3.26 (s, 3H), 3.22 (d, J=10.3 Hz, 1H), 2.73 (s, 1H), 2.32 (dt, J=12.2, 6.1 Hz, 2H), 2.22-2.15 (m, 1H), 1.97 (s, 3H), 1.95 (s, 2H), 1.87 (s, 3H), 1.76 (d, J=7.3 Hz, 1H), 1.63-1.57 (m, 3H), 1.52 (dd, J=10.8, 5.4 Hz, 1H), 1.19 (s, 2H), 1.19-1.12 (m, 2H), 1.09 (d, J=6.7 Hz, 3H), 1.05 (dd, J=7.1, 3.5 Hz, 6H), 0.92 (d, J=6.5 Hz, 3H), 0.87-0.82 (m, 3H), 0.82 (d, J=6.9 Hz, 3H). ¹³C NMR (151 MHz, 278 K, CDCl₃) δ 166.6, 142.1, 141.9, 139.6, 133.3, 132.2, 130.8, 130.6, 127.9, 127.1, 123.0, 99.5, 81.3, 79.7, 75.5, 75.2, 74.3, 70.5, 70.0, 59.1, 55.7, 44.7, 43.5, 43.3, 43.0, 41.3, 36.9, 36.3, 34.6, 22.8, 21.7, 17.8, 16.8, 16.4, 14.2, 13.2, 11.7, 9.3, 7.1. LC-MS (IT): calcd for C₃₉H₆₄O₁₀ [M+Fa−H]⁻ 737.5. found, 737.5.

### Comparative Example 22: Synthesis of Concanamycin X

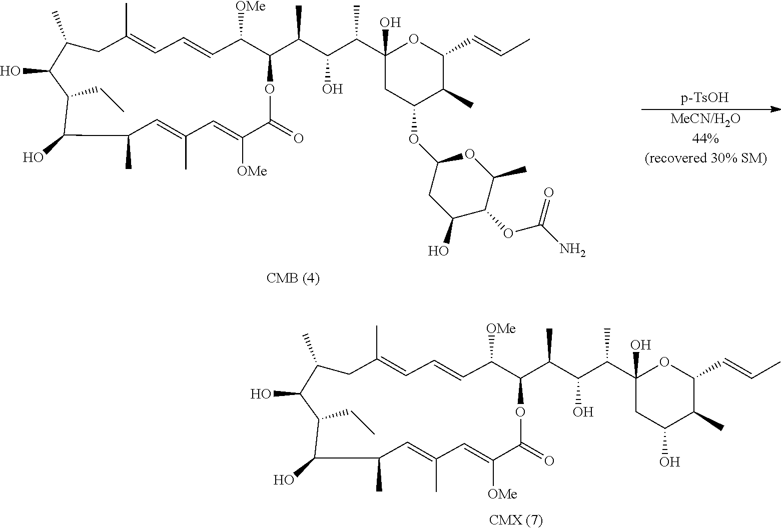

Concanamycin B (CMA; 80. mg, 94 μmol) was dissolved in MeCN (6.6 mL) and water (1.32 mL) before the addition of pTsOH (35.7 mg, 188 μmol, 2 eq). The reaction was ran overnight (between 15-20 hrs) before being cooled to 0° C. and quenched with a saturated solution of NaHCO₃. The crude product was extracted with CHCl3 (×3), washed with water, dried over Na2SO4, filtered and concentrated. The crude material was purified by flash column chromatography using 4-12% isopropanol in a 25% hexanes/chloroform solution. To fully wash the column, it was flushed with 100% isopropanol. The material collected was further purified by pTLC with 4% isopropanol/chloroform to yield the product. Concanamycin X ¹H NMR (600 MHz, CDCl₃) δ 6.59-6.51 (m, 1H), 6.36 (d, J=72.1 Hz, 1H), 5.93-5.71 (m, 2H), 5.66 (d, J=9.3 Hz, 1H), 5.55 (dt, J=15.0, 6.6 Hz, 1H), 5.28 (dt, J=14.9, 6.9 Hz, 1H), 5.21 (dd, J=15.0, 9.4 Hz, 1H), 5.04 (dd, J=41.4, 9.4 Hz, 1H), 4.72-4.38 (m, 1H), 4.04 (dt, J=12.5, 6.3 Hz, 1H), 3.97 (t, J=9.5 Hz, 2H), 3.85 (t, J=9.4 Hz, 1H), 3.73 (td, J=10.9, 4.9 Hz, 1H), 3.56 (s, 3H), 3.26 (d, J=5.2 Hz, 3H), 3.39-3.06 (m, 1H), 2.89 (t, J=7.9 Hz, 1H), 2.33 (dd, J=12.1, 4.7 Hz, 1H), 2.19 (dq, J=14.0, 7.3 Hz, 1H), 2.12 (t, J=8.0 Hz, 1H), 2.01 (d, J=37.0 Hz, 4H), 1.83 (d, J=61.9 Hz, 3H), 1.75 (d, J=7.5 Hz, 4H), 1.59 (d, J=6.5 Hz, 3H), 1.22 (d, J=6.2 Hz, 3H), 1.19 (d, J=11.2 Hz, 2H), 1.05 (p, J=7.4, 5.9 Hz, 6H), 0.91 (d, J=6.4 Hz, 3H), 0.83 (dt, J=20.7, 10.1 Hz, 7H). ¹³C NMR (151 MHz, CDCl₃) δ 166.4, 142.6, 142.1, 139.6, 133.8, 133.0, 130.9, 129.8, 128.1, 127.6, 125.8, 123.7, 99.7, 84.6, 81.3, 80.7, 75.4, 75.3, 70.6, 70.2, 64.6, 59.5, 59.2, 55.9, 45.4, 44.9, 43.7, 43.4, 43.2, 41.4, 37.3, 36.8, 36.5, 31.6, 25.5, 18.0, 17.7, 17.2, 16.3, 15.9, 14.9, 14.3, 13.4, 9.4, 7.2. LC-MS (IT): calcd for C₃₁H₆₂O₁₀ [M+Fa−H]⁻ 723.4. found, 723.4.

### Comparative Example 23: Synthesis of 21-deoxy CMF & 21-deoxy-16-hydroxy CMF (S24 and 25)

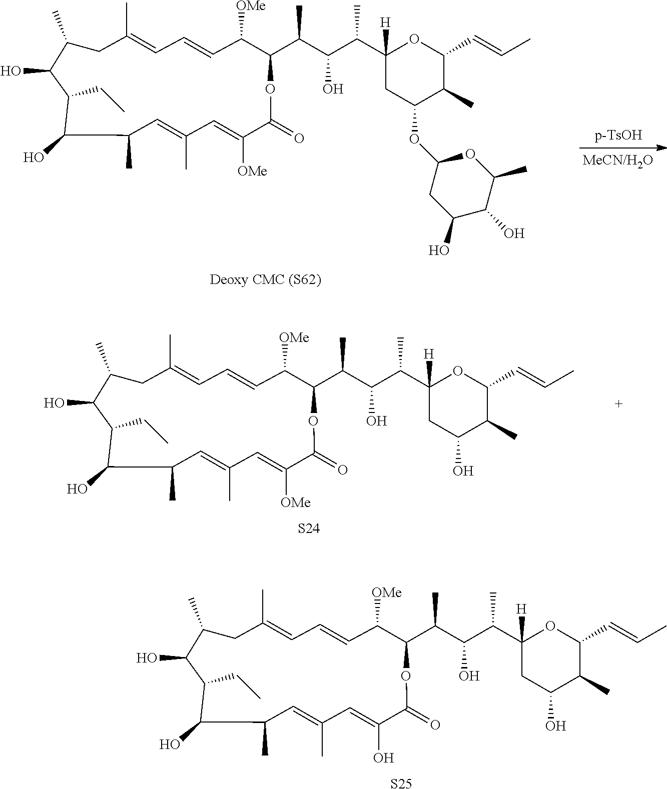

MeCN (11.6 mL) was added to a vial containing a mixture of 21-deoxy CMC (140 mg 165 μmol) before water (2.32 mL) was added. The reaction was stirred and pTsOH (72.0 mg, 379 μmol) was added before heating to 38° C. The reaction was run overnight (~16 hrs) then cooled to 0° C. Sodium bicarbonate was added to the reaction mix to basify and before concentrating. The water mix that was left over was washed with chloroform 3 times. The organics were combined, washed with brine, dried over sodium sulfate, filtered, and concentrated. The crude, yellow foam was first purified by FCC (12% acetone/DCM) and fractions 12/13 were collected and found to contain majority of the product. Starting material was also recovered when the gradient was increased to 100% acetone. Fractions 12/13 were further purified by pTLC (12% acetone/DCM) to yield 21-deoxy concanamycin F (CMF) and 21-deoxy-16-hydroxy-Concanamycin F. 21-Deoxy CMF ¹H NMR (600 MHz, CDCl₃) δ 6.53 (q, J=15.5, 14.2 Hz, 1H), 6.35 (d, J=38.6 Hz, 1H), 5.89-5.75 (m, 1H), 5.67 (d, J=9.8 Hz, 1H), 5.56 (dt, J=14.3, 7.3 Hz, 1H), 5.37-5.30 (m, 1H), 5.28-5.21 (m, 1H), 5.20 (d, J=8.5 Hz, 1H), 3.83 (q, J=10.6, 9.8 Hz, 2H), 3.75 (s, 1H), 3.62 (d, J=10.4 Hz, 1H), 3.58 (s, 3H), 3.49 (d, J=10.3 Hz, 1H), 3.43-3.36 (m, 1H), 3.33 (t, J=8.9 Hz, 1H), 3.26 (d, J=2.8 Hz, 3H), 3.22 (d, J=10.6 Hz, 1H), 2.72 (t, J=8.9 Hz, 1H), 2.28 (s, 1H), 2.11 (dt, J=17.8, 9.7 Hz, 2H), 2.05-1.93 (m, 5H), 1.86 (s, 3H), 1.69 (s, 1H), 1.62 (d, J=6.6 Hz, 3H), 1.51 (s, 1H), 1.31-1.19 (m, 2H), 1.20-1.15 (m, 2H), 1.13-1.06 (m, 3H), 1.05 (d, J=6.9 Hz, 3H), 0.93-0.81 (m, 12H). ¹³C NMR (151 MHz, CDCl₃) δ 165.9, 142.2, 141.6, 139.0, 132.8, 132.2, 130.4, 130.1, 128.5, 127.5, 123.1, 82.5, 82.0, 79.6, 76.8, 76.0, 74.5, 74.2, 69.2, 59.2, 55.8, 44.6, 43.8, 43.4, 39.2, 38.8, 37.4, 36.5, 34.5, 22.9, 21.5, 17.8, 16.9, 16.5, 14.2, 13.4, 11.7, 9.6, 8.2. LC-MS (IT): calcd for C₃₉H₆₄O₉[M+Fa−H]⁻ 721.5. found, 721.4. 21-Deoxy-16-hydroxy-CMF ¹H NMR (600 MHz, CDCl₃) δ 6.43 (d, J=3.4 Hz, 1H), 6.32 (t, J=13.1 Hz, 1H), 5.94 (d, J=9.7 Hz, 1H), 5.79 (d, J=10.8 Hz, 1H), 5.70 (h, J=5.4 Hz, 1H), 5.43 (dd, J=15.9, 7.8 Hz, 2H), 5.09 (q, J=12.2, 9.0 Hz, 1H), 4.60 (d, J=9.8 Hz, 1H), 3.84 (d, J=10.3 Hz, 1H), 3.64 (d, J=23.2 Hz, 4H), 3.41 (dq, J=23.9, 13.1, 11.3 Hz, 3H), 3.25 (d, J=9.9 Hz, 1H), 2.68-2.62 (m, 1H), 2.33 (s, 1H), 2.21-2.14 (m, 2H), 2.14 (s, 1H), 1.95 (s, 3H), 1.90-1.76 (m, 2H), 1.73 (d, J=6.1 Hz, 3H), 1.67 (s, 3H), 1.54 (s, 1H), 1.39 (s, 1H), 1.25 (d, J=55.4 Hz, 4H), 1.14 (t, J=4.5 Hz, 3H), 1.04 (d, J=6.1 Hz, 3H), 0.99 (d, J=6.6 Hz, 2H), 0.97-0.87 (m, 9H). ¹³C NMR (151 MHz, CDCl₃) δ 164.5, 142.2, 140.1, 137.7, 132.4, 130.8, 130.2, 129.2, 128.5, 128.0, 121.7, 82.3, 81.8, 81.7, 78.6, 78.4, 77.5, 77.0, 74.1, 59.8, 45.1, 43.7, 42.1, 40.9, 39.5, 38.7, 38.2, 35.0, 24.6, 19.2, 18.2, 18.0, 16.8, 14.4, 13.6, 13.3, 12.2, 8.2. LC-MS (QTOF): calcd for C₃₈H₆₄O₉[M+H]⁺ 663.45. found, 663.45.

### Comparative Example 24: Synthesis of 3-nonanoate Concanamycin B (11)

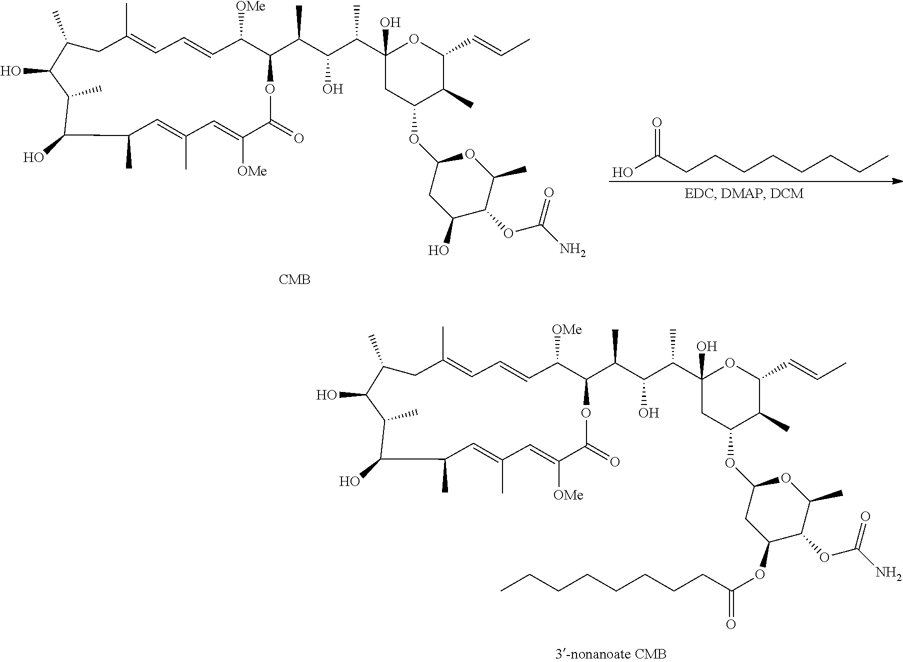 [SMILES: C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](C)[C@@H](O)[C@H](C)C/C(C)=C/C=C/[C@@H]2OC)C[C@@H](O[C@@H]2C[C@H](O)[C@@H](OC(N)=O)[C@H](C)O2)[C@@H]1C ; CCCCCCCCC(=O)O ; C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](C)[C@@H](O)[C@H](C)C/C(C)=C/C=C/[C@@H]2OC)C[C@@H](O[C@@H]2C[C@H](OC(=O)CCCCCCCC)[C@@H](OC(N)=O)[C@H](C)O2)[C@@H]1C]

CMB (30 mg, 35 μmol), nonanoic acid (12 μL, 70, μmol, 2.0 eq), DMAP (9.0 mg, 74 μmol, 2.1 eq), EDC (15 mg, 78 μmol, 2.2 eq), DCM (5 mL). 3′-nonanoate CMB ¹H NMR (600 MHz, CDCl₃) δ 6.54 (h, J=14.0, 13.0 Hz, 1H), 6.34 (d, J=72.0 Hz, 1H), 5.87 (d, J=12.3 Hz, 1H), 5.81 (d, J=19.8 Hz, 1H), 5.64 (d, J=9.9 Hz, OH), 5.53 (dt, J=14.9, 6.8 Hz, 1H), 5.26 (dd, J=15.5, 7.7 Hz, 1H), 5.19 (dd, J=15.0, 9.4 Hz, 1H), 5.03 (dd, J=41.1, 9.4 Hz, 1H), 4.93 (td, J=10.8, 5.2 Hz, 1H), 4.75 (s, 1H), 4.70 (s, 1H), 4.63-4.56 (m, 2H), 4.06-3.92 (m, 2H), 3.84 (t, J=9.4 Hz, 1H), 3.76 (td, J=11.1, 4.8 Hz, 1H), 3.55 (s, 3H), 3.39 (dd, J=9.7, 6.1 Hz, 1H), 3.25 (d, J=5.4 Hz, 3H), 2.87 (t, J=7.7 Hz, 1H), 2.32 (td, J=13.7, 12.5, 6.1 Hz, 1H), 2.28 (t, J=7.6 Hz, 2H), 2.19 (dq, J=12.5, 7.3, 6.3 Hz, 2H), 2.11 (d, J=13.5 Hz, 1H), 2.02 (s, 2H), 1.96 (s, 2H), 1.86 (d, J=12.2 Hz, 2H), 1.75 (d, J=15.0 Hz, 3H), 1.67 (q, J=11.1 Hz, 1H), 1.57 (d, J=6.5 Hz, 5H), 1.23 (dd, J=13.4, 6.6 Hz, 19H), 1.11 (d, J=8.5 Hz, 1H), 1.03 (dd, J=23.3, 6.9 Hz, 5H), 0.94 (d, J=7.2 Hz, 2H), 0.86 (t, J=6.9 Hz, 7H), 0.82 (d, J=6.7 Hz, 3H).

### Comparative Example 25: Synthesis of 3′-pent-4-ynoate Concanamycin A

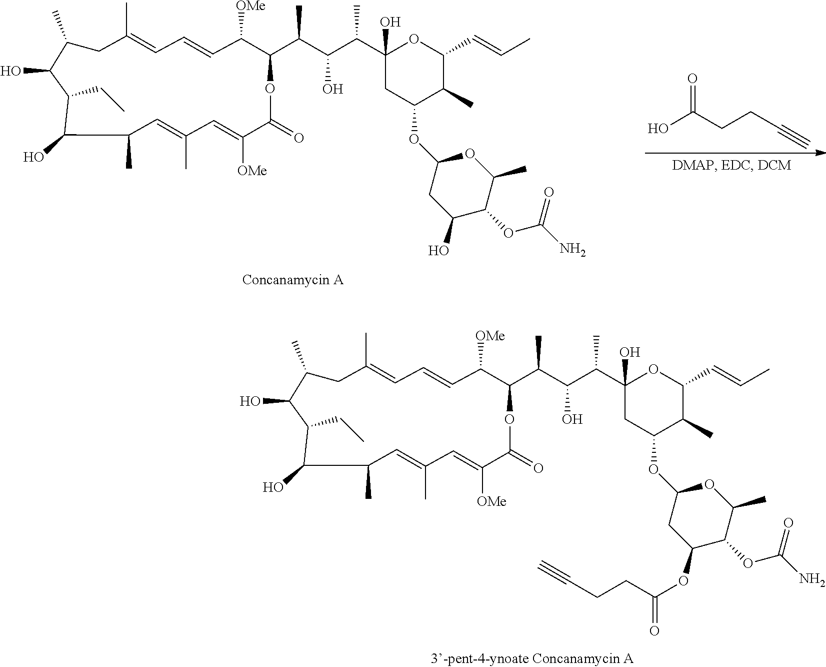

### Comparative Example 26: Synthesis of 3′-pent-4-ynoate Concanamycin B

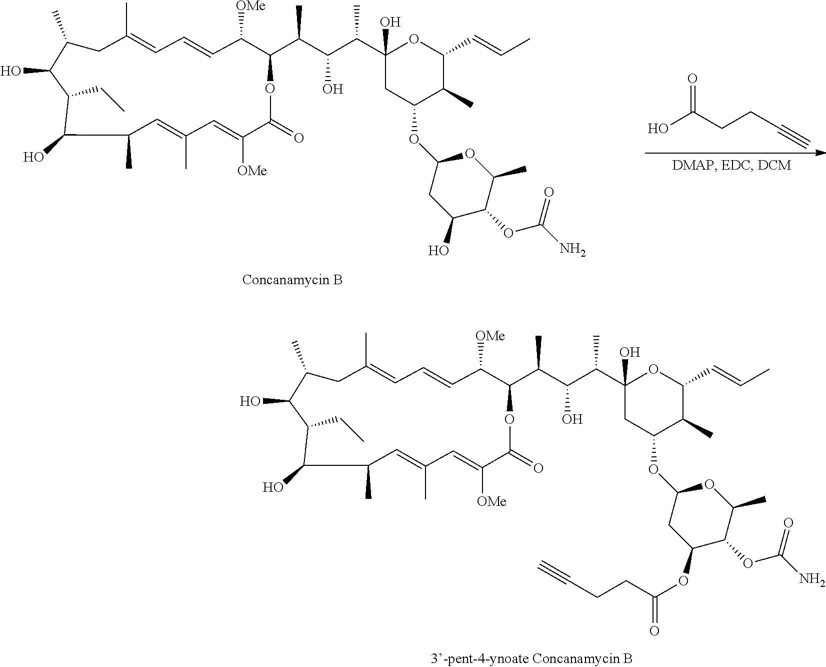

### Comparative Example 27: Synthesis of 3′-pent-4-ynoate Concanamycin C

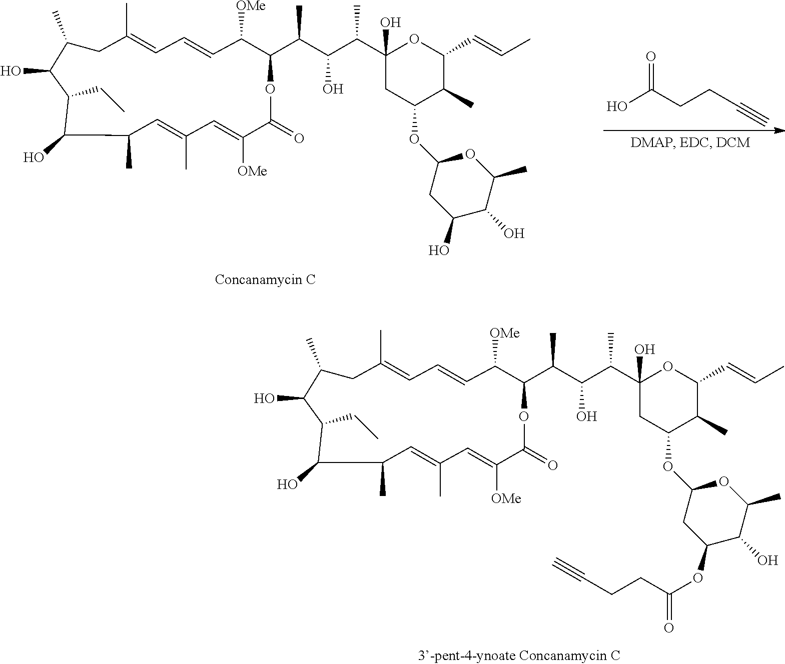

### EXAMPLES

### Example 1: Bafilomycin Esterification General Procedure

DMAP, EDC, and carboxylic acid were added to a solution of bafilomycin A₁ in DCM under anhydrous conditions. The reaction was stirred overnight then concentrated under reduced pressure. The crude reaction mix was purified by preparative TLC (pTLC).

### Example 2: 21-Formyl Bafilomycin A₁ (Compound A1, Also Called S61)

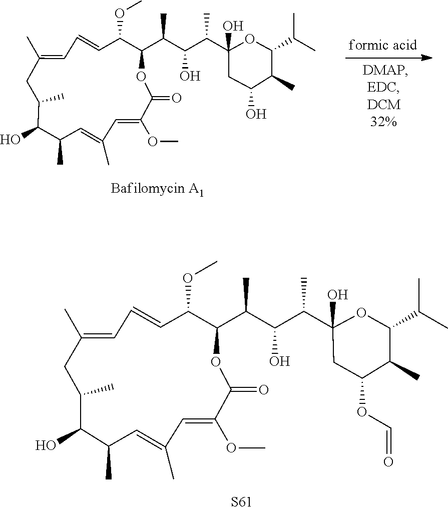 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(O)C[C@@H](O)[C@H](C)[C@@H](C(C)C)O2)OC1=O ; CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(O)C[C@@H](OC=O)[C@H](C)[C@@H](C(C)C)O2)OC1=O ; "S61"]

Amounts: DMAP (5.1 mg, 42.0 μmol, 2.1 eq), EDC (8.5 mg, 44 μmol, 2.2 eq), formic acid (10.95 μL, 25 μmol, 1.25 eq), bafilomycin A₁ (12 mg, 20 μmol), and DCM (2.5 mL). Purified by pTLC (20% acetone in hexanes) to yield the product. S36 ¹H NMR (599 MHz, Acetone) δ 8.17 (d, J=1.2 Hz, 1H), 6.71 (s, 1H), 6.68 (dd, J=15.0, 10.8 Hz, 1H), 5.96 (d, J=8.8 Hz, 1H), 5.80 (d, J=10.7 Hz, 1H), 5.43 (t, J=1.6 Hz, 1H), 5.14 (dd, J=15.0, 9.2 Hz, 1H), 5.02 (td, J=10.9, 4.8 Hz, 1H), 4.96 (dq, J=8.5, 1.5 Hz, 1H), 4.78 (d, J=4.3 Hz, 1H), 4.18 (ddt, J=9.4, 5.2, 1.7 Hz, 1H), 4.09-4.03 (m, 2H), 3.64 (s, 3H), 3.60 (dd, J=10.3, 2.1 Hz, 1H), 3.31 (tt, J=5.1, 2.3 Hz, 1H), 3.24 (s, 3H), 2.55 (dt, J=8.9, 7.0 Hz, 1H), 2.28 (ddd, J=11.7, 4.9, 1.3 Hz, 1H), 2.21-2.13 (m, 1H), 2.10-1.99 (m, 2H), 1.98 (s, 3H), 1.93 (d, J=1.4 Hz, 3H), 1.93-1.86 (m, 2H), 1.84 (t, J=7.4 Hz, 1H), 1.62-1.53 (m, 1H), 1.29 (s, 1H), 1.05 (dd, J=7.1, 1.3 Hz, 3H), 1.00 (dd, J=7.2, 1.3 Hz, 3H), 0.95 (dd, J=6.9, 1.3 Hz, 3H), 0.92 (dd, J=6.8, 1.3 Hz, 3H), 0.88 (dd, J=6.9, 1.3 Hz, 3H), 0.83 (dd, J=6.5, 1.3 Hz, 3H), 0.81 (dd, J=6.7, 1.4 Hz, 3H). ¹³C NMR (151 MHz, Acetone) δ 167.84, 161.73, 145.74, 144.84, 141.94, 134.47, 134.24, 132.79, 127.04, 125.24, 99.72, 83.34, 80.43, 77.42, 76.50, 74.26, 71.59, 60.18, 55.67, 42.89, 42.27, 41.71, 40.87, 38.86, 38.28, 38.01, 28.75, 22.25, 21.62, 20.37, 17.73, 14.58, 14.16, 12.48, 10.29, 7.37.

### Example 3: Synthesis of 21-(2-fluoro)acetate Baf (Compound A2)

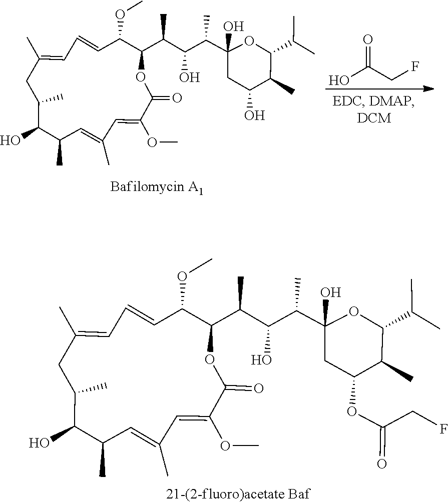

Amounts: DMAP (4.31 mg, 35 μmol, 2.1 eq), EDC (7.1 mg, 37 μmol, 2.2 eq), fluoroacetic acid (1.9 μL, 34 μM, 2.0 eq), bafilomycin A1 (10. mg, 17 μmol), and DCM (2.1 mL). Purified by pTLC (20% acetone in hexanes) to yield the product. 21-(2-fluoro)acetate Baf 1H NMR (599 MHz, Acetone) δ 6.76-6.72 (m, 1H), 6.74-6.66 (m, 1H), 5.99 (d, J=8.9 Hz, 1H), 5.83 (d, J=10.8 Hz, 1H), 5.48-5.44 (m, 1H), 5.20-5.13 (m, 1H), 5.11-5.06 (m, 1H), 5.06 (s, 1H), 5.06-4.90 (m, 3H), 4.80 (dt, J=4.5, 1.3 Hz, 1H), 4.20 (d, J=10.2 Hz, 1H), 4.08 (tt, J=8.1, 1.9 Hz, 2H), 3.68-3.65 (m, 3H), 3.63 (dd, J=10.5, 2.4 Hz, 1H), 3.33 (q, J=4.7, 2.9 Hz, 1H), 3.30-3.24 (m, 3H), 2.62-2.53 (m, 1H), 2.34 (ddd, J=11.9, 5.1, 2.0 Hz, 1H), 2.22-2.16 (m, 1H), 2.01 (q, J=1.3 Hz, 3H), 2.00-1.94 (m, 3H), 1.90 (s, 2H), 1.95-1.84 (m, 1H), 1.61 (dt, J=12.8, 5.3 Hz, 2H), 1.09-1.06 (m, 3H), 1.03-1.00 (m, 3H), 0.99-0.96 (m, 3H), 0.94 (dt, J=6.8, 1.4 Hz, 3H), 0.92-0.88 (m, 3H), 0.88-0.84 (m, 3H), 0.84-0.81 (m, 3H). 13C NMR (151 MHz, Acetone) δ 168.22 (d, J=21.7 Hz), 167.83, 145.74, 144.84, 141.95, 134.47, 134.23, 132.79, 127.03, 125.24, 99.77, 83.34, 80.42, 78.46 (d, J=178.2 Hz), 77.41, 76.51, 75.68, 71.59, 60.18, 55.67, 42.91, 42.27, 41.71, 40.65, 38.96, 38.28, 38.01, 28.72, 22.25, 21.60, 20.36, 17.73, 14.57, 14.16, 12.48, 10.29, 7.37.

### Example 4: Synthesis of 21-(2-chloro)acetate Baf (Compound A3)

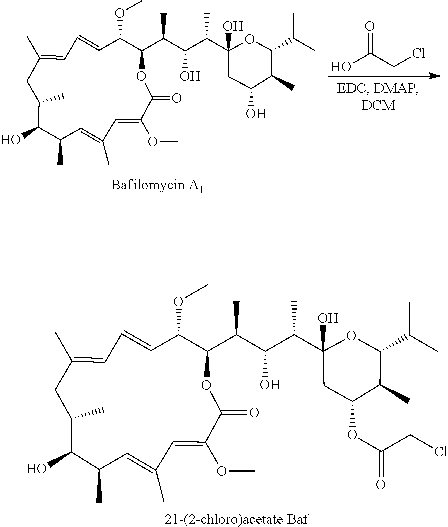

Synthesized in an analogous fashion to 21-(2-fluoro)acetate Baf (Compound A2).

### Example 5: Synthesis of 21-(2-bromo)acetate Baf (Compound A4)

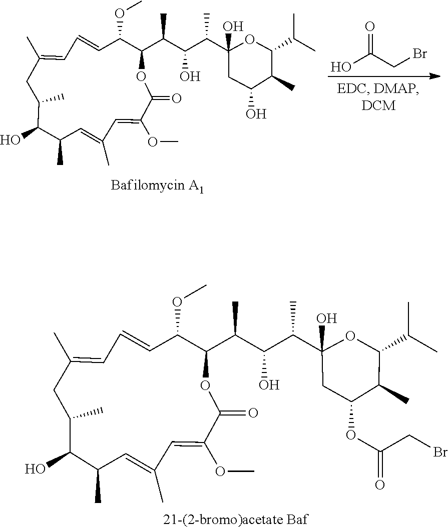

21-(2-bromo)acetate Baf ¹H NMR (599 MHz, Acetone) δ 6.75-6.66 (m, 2H), 5.98 (d, J=8.9 Hz, 1H), 5.82 (d, J=11.0 Hz, 1H), 5.47-5.41 (m, 1H), 5.17 (dd, J=15.1, 9.1 Hz, 1H), 5.05-4.96 (m, 2H), 4.80 (d, J=4.3 Hz, 1H), 4.34-4.28 (m, 1H), 4.30-4.23 (m, 1H), 4.19 (t, J=8.8 Hz, 1H), 4.14-4.02 (m, 2H), 3.69-3.64 (m, 3H), 3.62 (d, J=10.0 Hz, 1H), 3.33 (ddd, J=5.5, 3.3, 2.1 Hz, 3H), 3.29-3.24 (m, 3H), 2.62-2.52 (m, 1H), 2.33 (dt, J=11.7, 3.0 Hz, 1H), 2.03 (d, J=7.3 Hz, 1H), 2.00 (q, J=1.2 Hz, 3H), 1.95 (s, 4H), 1.89 (dd, J=17.8, 9.7 Hz, 2H), 1.61 (td, J=10.4, 5.3 Hz, 1H), 1.07 (dt, J=7.1, 1.2 Hz, 3H), 1.01 (dt, J=7.2, 1.2 Hz, 3H), 0.98-0.95 (m, 3H), 0.94 (dt, J=6.7, 1.2 Hz, 3H), 0.90 (dt, J=6.9, 1.2 Hz, 3H), 0.86 (dt, J=6.6, 1.2 Hz, 3H), 0.84-0.82 (m, 3H). ¹³C NMR (151 MHz, Acetone) δ 167.61, 145.75, 144.84, 141.94, 134.47, 134.23, 132.79, 127.03, 125.24, 99.78, 99.68, 83.34, 80.35 (d, J=20.5 Hz), 77.41, 76.53 (d, J=8.7 Hz), 71.53 (d, J=18.4 Hz), 60.18, 55.67, 52.38 (d, J=780.9 Hz), 42.90 (d, J=4.1 Hz), 42.27, 41.86, 41.71, 40.54, 38.99, 38.28, 38.00 (d, J=3.2 Hz), 28.71, 22.25, 21.60, 20.36, 17.73, 14.57, 14.16, 12.45, 10.29, 7.38.

### Example 6: Synthesis of 21-(2-(S)-bromo)propanoate Baf (Compound A5)

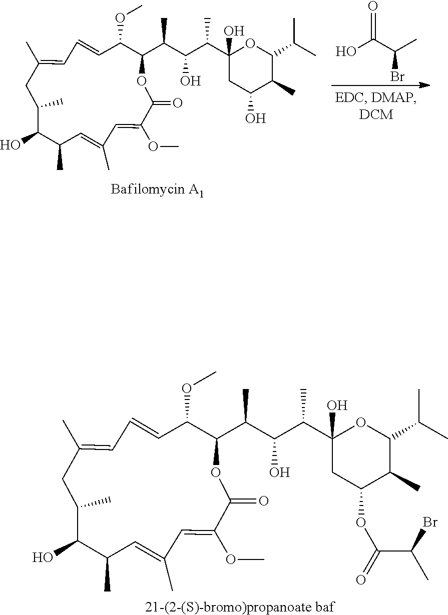

21-(2-(S)-bromo)propanoate Baf 1H NMR (599 MHz, Acetone) δ 6.74 (q, J=0.9 Hz, 1H), 6.74-6.67 (m, 1H), 5.99 (d, J=8.8 Hz, 1H), 5.86-5.81 (m, 1H), 5.45 (t, J=1.9 Hz, 1H), 5.17 (dd, J=15.1, 9.2 Hz, 1H), 5.04-4.96 (m, 2H), 4.80 (dt, J=4.4, 1.4 Hz, 1H), 4.59 (qd, J=6.9, 1.6 Hz, 1H), 4.24-4.18 (m, 1H), 4.14-4.06 (m, 2H), 3.71-3.63 (m, 3H), 3.63 (dd, J=10.4, 2.0 Hz, 1H), 3.33 (ddd, J=7.1, 4.7, 1.9 Hz, 1H), 3.31-3.25 (m, 3H), 2.58 (dtd, J=9.2, 7.2, 5.3 Hz, 1H), 2.35-2.27 (m, 1H), 2.23-2.15 (m, 1H), 2.08-2.02 (m, 1H), 2.03-1.96 (m, 3H), 1.98-1.94 (m, 3H), 1.91 (s, 1H), 1.88 (q, J=7.2 Hz, 1H), 1.84-1.79 (m, 3H), 1.74-1.60 (m, 1H), 1.34-1.25 (m, 4H), 1.08 (dd, J=7.1, 1.6 Hz, 3H), 1.04-1.00 (m, 3H), 0.98 (dd, J=7.0, 1.7 Hz, 3H), 0.95 (dd, J=6.8, 1.6 Hz, 3H), 0.91 (dt, J=7.0, 2.0 Hz, 6H), 0.84 (dd, J=6.9, 1.7 Hz, 3H). 13C NMR (151 MHz, Acetone) δ 170.28, 167.84, 145.74, 144.84, 141.95, 134.47, 134.23, 132.80, 127.03, 125.24, 99.76, 83.34, 80.42, 77.42, 76.54, 76.13, 71.59, 60.18, 55.67, 42.91, 42.28, 41.73 (d, J=2.8 Hz), 40.47, 39.14, 38.28, 38.01, 28.67, 22.25, 21.94, 21.61, 20.37, 17.73, 14.58, 14.16, 12.48, 10.29, 7.40.

### Example 7: Synthesis of 3-one CMA (Compounds A6 and A7, Also Called S47 and S48)

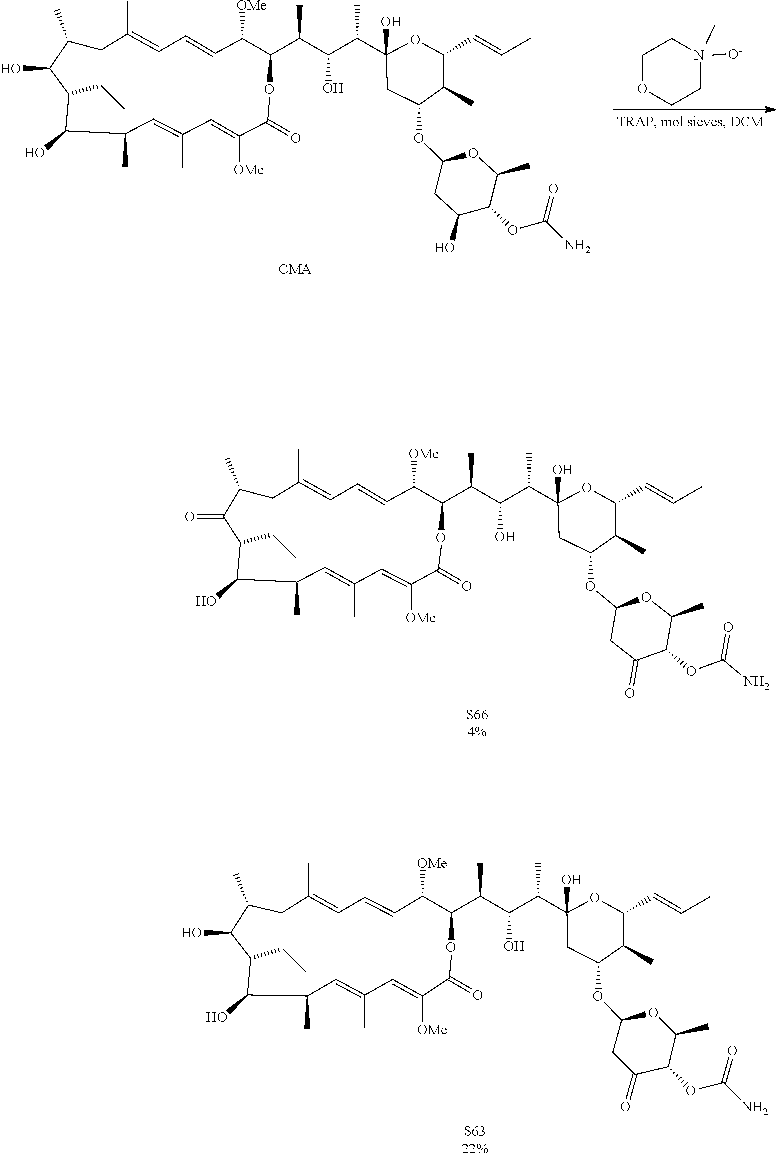

CMA (49 mg, 57 μmol) was dissolved in dry DCM (3.0 mL) and activated molecular sieves (4A, 8-12 mesh) were added. 4-Methylmorpholine N-oxide monohydrate (31 mg) was added to the reaction, followed by TPAP ( ) in two increments. The mixture was stirred at RT for 2 hours, with the solution changing from a grey-green to black over the course. To remove some of the catalyst, the reaction mixture was filtered over a bed of celite. Residual catalyst was removed upon flash column chromatography (5%-40% IPA in a hexane/CHCl₃ mixture (25% Hexane in CHCl₃) to yield S66 and S63. S66 and S63 were further purified separately by pTLC (5% IPA/CHCl₃) to yield the product. S63 ¹H NMR (599 MHz, 278 K, CDCl₃) δ 6.56 (dd, J=15.1, 10.7 Hz, 1H), 6.40 (s, 1H), 5.89 (s, 1H), 5.78 (d, J=10.7 Hz, 1H), 5.68 (d, J=9.6 Hz, 1H), 5.57 (dd, J=15.1, 6.8 Hz, 1H), 5.29 (dd, J=15.1, 8.3 Hz, 1H), 5.22 (dd, J=15.1, 9.0 Hz, 1H), 5.01 (d, J=9.1 Hz, 1H), 4.82 (d, J=10.1 Hz, 1H), 4.75 (dd, J=7.2, 4.7 Hz, 1H), 4.70 (d, J=4.1 Hz, 1H), 4.00 (q, J=9.6, 8.7 Hz, 2H), 3.89-3.80 (m, 3H), 3.63-3.56 (m, 1H), 3.57 (s, 3H), 3.26 (s, 3H), 3.25-3.20 (m, 1H), 2.77-2.68 (m, 3H), 2.30 (dd, J=12.0, 4.8 Hz, 2H), 2.18 (s, 1H), 1.98 (s, 5H), 1.87 (s, 3H), 1.76 (d, J=7.2 Hz, 2H), 1.59 (d, J=6.5 Hz, 4H), 1.51 (s, 1H), 1.39 (d, J=6.0 Hz, 3H), 1.35 (d, J=6.1 Hz, 1H), 1.13 (t, J=10.6 Hz, 2H), 1.09 (d, J=6.7 Hz, 3H), 1.04 (d, J=7.1 Hz, 6H), 0.93 (d, J=6.4 Hz, 4H), 0.87 (dt, J=16.5, 7.1 Hz, 3H), 0.82 (d, J=6.8 Hz, 3H). ¹³C NMR (151 MHz, 278 K, CDCl₃) δ 199.57, 165.63, 154.18, 141.12, 140.79, 138.60, 132.29, 131.18, 129.69, 129.11, 126.97, 126.08, 121.95, 98.48, 96.04, 80.25, 78.63, 77.91, 75.52, 74.53, 74.41, 73.26, 69.06, 68.64, 58.05, 54.73, 47.55, 43.69, 42.29, 40.24, 40.15, 38.57, 35.81, 35.31, 33.54, 21.71 (d, J=6.9 Hz), 20.65, 17.41, 16.79, 15.76, 15.37, 13.17, 12.41, 10.63, 8.28, 6.10.

### Example 8: Concanamycin Esterification General Procedure

DMAP, EDC, and carboxylic acid were added to a solution of concanamycin of choice in DCM under anhydrous conditions. After stirring for 30 minutes a TLC was taken (5% IPA in CHCl₃) and every 15 minutes afterwards. Once the TLC showed two distinct spots above the starting material, the reaction was concentrated (usually <2.5 hrs total). The crude material was first purified by flash column chromatography, yielding 2 products and purified residual starting material (~40% IPA/15% Hex/55% CHCl₃). The two products were further purified by pTLC.

### Example 9: 3′-Acetyl Concanamycin B (Compound A8, Also Called 24) & 3′,9-Diacetyl CMB (Compound A9, Also Called 25)

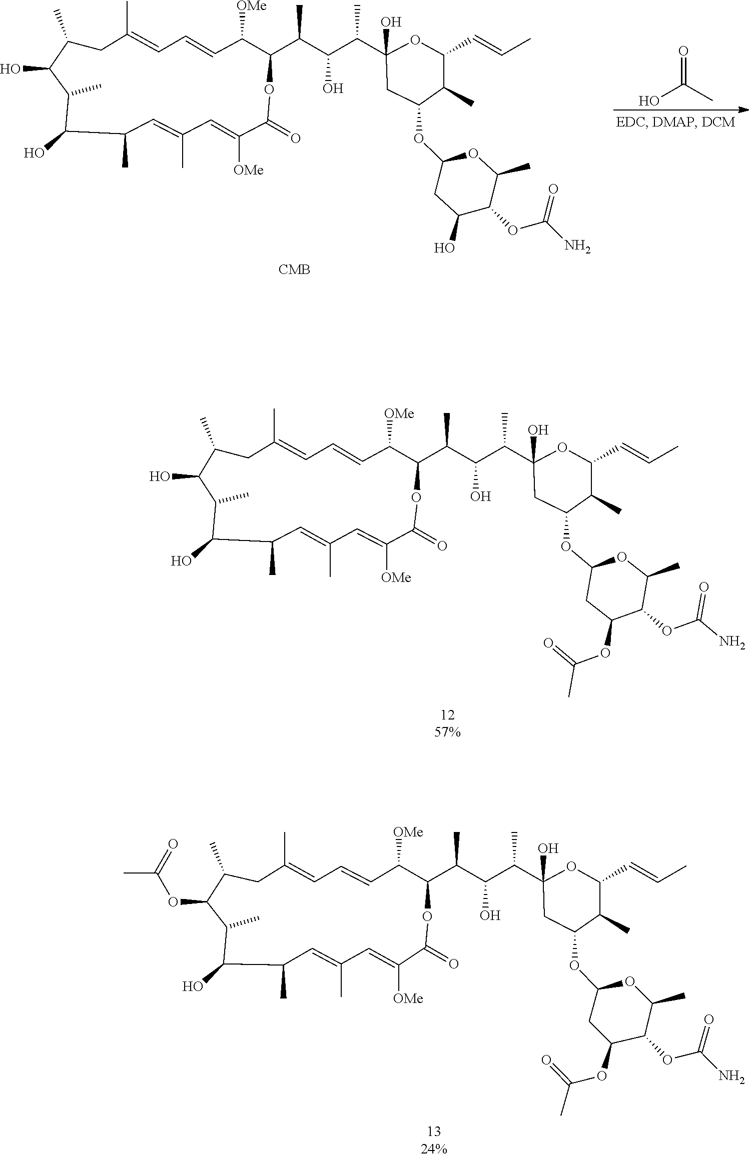

CMB (30 mg, 35 μmol), acetic acid (3.0 μL, 53 μmol, 1.5 eq), DMAP (9.0 mg, 74 μmol, 2.1 eq), EDC (14.8 mg, 78 μmol, 2.2 eq), DCM (5.0 mL). isolated 12 and 13. Acetyl CMB ¹H NMR (600 MHz, CDCl₃) δ 6.62-6.48 (m, 1H), 6.36 (d, J=71.2 Hz, 1H), 5.91-5.78 (m, 2H), 5.66 (d, J=10.0 Hz, 1H), 5.55 (dt, J=14.8, 6.7 Hz, 1H), 5.28 (dt, J=15.3, 7.3 Hz, 1H), 5.21 (dd, J=15.0, 9.4 Hz, 1H), 5.04 (dd, J=40.8, 9.5 Hz, 1H), 4.94 (ddd, J=11.6, 9.4, 5.2 Hz, 1H), 4.60 (q, J=9.6, 9.1 Hz, 2H), 4.44 (d, J=4.3 Hz, 1H), 3.98 (q, J=14.2, 9.4 Hz, 2H), 3.85 (t, J=9.4 Hz, 1H), 3.77 (td, J=10.6, 4.4 Hz, 1H), 3.73-3.67 (m, 1H), 3.56 (s, 3H), 3.40 (dq, J=12.2, 6.2 Hz, 1H), 3.26 (d, J=5.1 Hz, 3H), 3.35-3.07 (m, 1H), 2.93-2.51 (m, 1H), 2.38 (q, J=7.6 Hz, 1H), 2.32 (dd, J=12.1, 4.7 Hz, 1H), 2.21 (dt, J=11.4, 6.1 Hz, 2H), 2.06 (s, 5H), 2.00 (d, J=36.7 Hz, 3H), 1.83 (d, J=62.0 Hz, 3H), 1.75 (d, J=7.2 Hz, 1H), 1.73-1.67 (m, 1H), 1.58 (d, J=6.5 Hz, 3H), 1.30-1.22 (m, 4H), 1.15-0.77 (m, 20H). ¹³C NMR (151 MHz, CDCl₃) δ 169.8, 165.1, 154.8, 141.5, 138.5, 134.6, 132.6, 131.8, 131.2, 130.0, 129.8, 128.5, 126.9, 126.2, 124.9, 124.7, 122.3, 98.5, 94.9, 83.4, 83.1, 80.1, 79.5, 75.0, 74.7, 74.5, 74.2, 74.0, 69.9, 69.0, 68.8, 58.4, 54.7, 44.2, 43.7, 40.3, 40.2, 38.7, 36.1, 35.9, 35.6, 35.3, 34.3, 33.8, 30.4, 21.0, 20.1, 18.0, 16.8, 16.5, 16.4, 15.7, 14.9, 14.7, 13.7, 13.2, 12.4, 11.6, 11.5, 8.3, 6.1. LC-MS (IT): calcd for C₄₇H₇₅NO₁₅ [M+Fa−H]⁻ 938.5. found, 938.5.. Diacetyl CMB ¹H NMR (600 MHz, CDCl₃) δ 6.59 (dd, J=15.0, 10.8 Hz, 1H), 6.40 (s, 1H), 5.90 (d, J=2.3 Hz, 1H), 5.86 (d, J=10.7 Hz, 1H), 5.60 (d, J=9.9 Hz, 1H), 5.55 (dq, J=13.1, 6.2 Hz, 1H), 5.28 (dd, J=15.3, 8.1 Hz, 1H), 5.23 (dd, J=15.0, 9.3 Hz, 1H), 4.99 (d, J=9.5 Hz, 1H), 4.94 (td, J=10.6, 5.1 Hz, 1H), 4.73 (d, J=4.3 Hz, 1H), 4.66 (d, J=11.0 Hz, 1H), 4.64-4.56 (m, 2H), 4.06-4.00 (m, 1H), 3.98 (t, J=9.2 Hz, 1H), 3.84 (t, J=9.4 Hz, 1H), 3.77 (td, J=10.8, 4.7 Hz, 1H), 3.61-3.57 (m, 1H), 3.59-3.55 (m, 3H), 3.40 (dq, J=12.1, 6.1 Hz, 1H), 3.25 (d, J=1.3 Hz, 3H), 2.68 (t, J=9.0 Hz, 1H), 2.55 (t, J=8.3 Hz, 1H), 2.32 (dd, J=12.1, 4.7 Hz, 1H), 2.22 (dq, J=12.0, 6.2, 4.5 Hz, 2H), 2.15-2.09 (m, 4H), 2.08-2.05 (m, 4H), 1.95 (s, 3H), 1.88 (s, 3H), 1.84 (dd, J=9.7, 6.9 Hz, 1H), 1.75 (dt, J=16.7, 8.5 Hz, 1H), 1.69 (q, J=11.3 Hz, 1H), 1.58 (d, J=6.4 Hz, 3H), 1.24 (d, J=7.3 Hz, 4H), 1.12 (t, J=11.6 Hz, 1H), 1.07 (d, J=7.1 Hz, 3H), 1.03 (d, J=6.9 Hz, 3H), 0.88 (d, J=6.4 Hz, 6H), 0.82 (d, J=6.9 Hz, 3H), 0.69 (d, J=7.1 Hz, 3H). ¹³C NMR (151 MHz, CDCl₃) δ 170.99, 170.77, 166.57, 155.75, 141.87, 141.72, 138.90, 133.72, 132.44, 130.94, 130.80, 127.90, 127.55, 123.73, 99.57, 95.92, 84.66, 81.05, 76.06, 75.47, 75.32, 75.02, 70.96, 69.99, 69.83, 59.00, 55.66, 45.90, 41.30, 41.19, 39.75, 37.01, 36.92, 36.48, 34.85, 33.21, 21.50, 21.15, 21.09, 17.81, 17.42, 16.90, 16.76, 16.04, 14.15, 13.40, 9.20, 7.14.

### Example 10: Synthesis of 3-pentanoate Concanamycin B & 3′,9-Dipentanoate Concanamycin B (Compounds A10 and A11, Also Called 21 and 22)

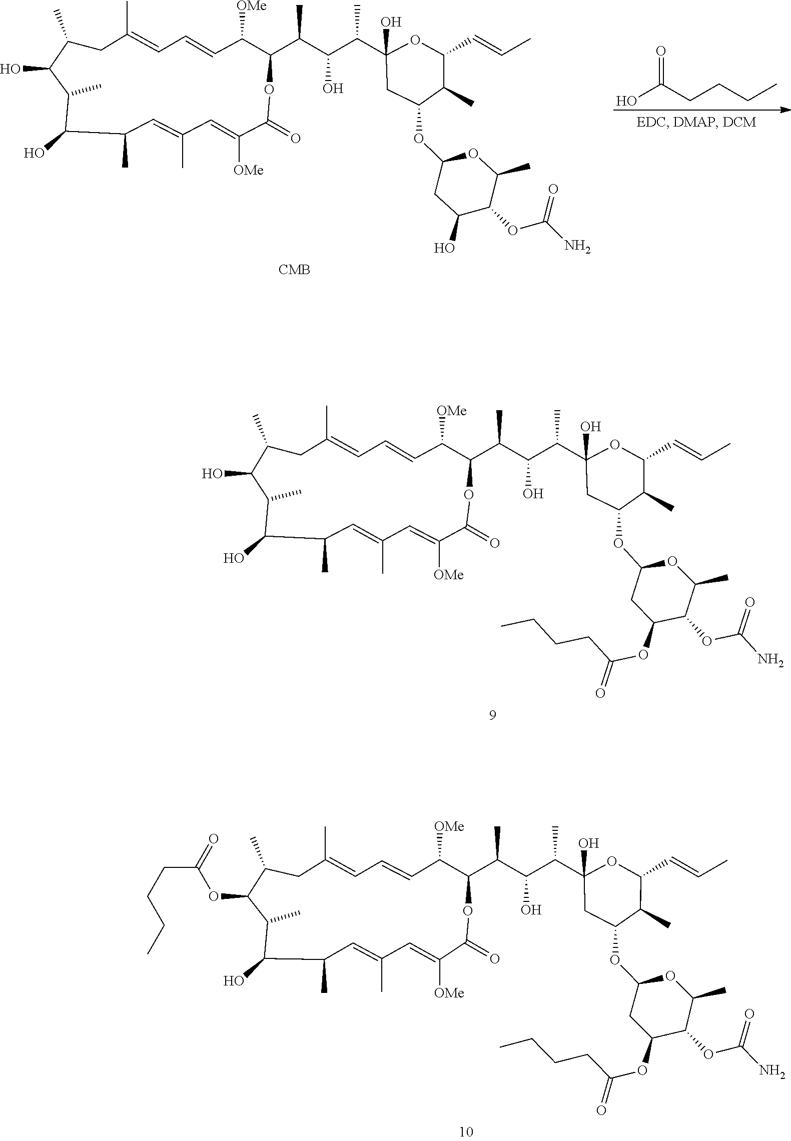

CMB (45 mg, 53 μmol), pentanoic acid (8.6 μL, 79 μmol, 1.5 eq), DMAP (14 mg, 111 μmol), EDC (22 mg, 116 μmol, 2.2 eq), DCM (7.5 mL). CMB pent ¹H NMR (599 MHz, CDCl₃) δ 6.57-6.49 (m, 1H), 6.34 (d, J=73.4 Hz, 1H), 5.86 (d, J=10.7 Hz, 1H), 5.83 (s, 1H), 5.64 (d, J=9.5 Hz, OH), 5.52 (dq, J=17.2, 6.3 Hz, 1H), 5.29-5.23 (m, 1H), 5.25-5.16 (m, 1H), 5.02 (dd, J=41.0, 9.4 Hz, 1H), 4.93 (ddd, J=11.8, 9.4, 5.2 Hz, 1H), 4.77 (s, 1H), 4.69 (s, 2H), 4.62-4.55 (m, 2H), 4.43 (s, 1H), 3.96 (q, J=9.5, 8.4 Hz, 2H), 3.84 (t, J=9.4 Hz, 1H), 3.75 (td, J=10.8, 4.7 Hz, 1H), 3.55 (s, 3H), 3.38 (dq, J=12.3, 6.2 Hz, 1H), 3.30 (d, J=8.8 Hz, 1H), 3.24 (d, J=4.6 Hz, 3H), 2.90-2.84 (m, 1H), 2.60 (s, 1H), 2.34-2.28 (m, 1H), 2.28 (t, J=7.5 Hz, 2H), 2.19 (dt, J=11.0, 5.7 Hz, 2H), 2.11 (d, J=12.6 Hz, 1H), 2.02 (s, 3H), 1.97 (d, J=11.5 Hz, 2H), 1.87 (s, 3H), 1.74 (s, 1H), 1.65 (ddd, J=19.0, 11.1, 8.4 Hz, 1H), 1.56 (p, J=7.7 Hz, 5H), 1.30 (h, J=7.4 Hz, 2H), 1.23 (s, 1H), 1.22 (d, J=6.1 Hz, 3H), 1.20 (d, J=6.2 Hz, 3H), 1.13-1.08 (m, 1H), 1.09-0.99 (m, 6H), 0.94 (d, J=7.1 Hz, 1H), 0.87 (q, J=6.5, 5.7 Hz, 6H), 0.86-0.76 (m, 4H). ¹³C NMR (151 MHz, CDCl₃) δ 173.66, 166.20, 155.92, 142.67, 142.48, 141.78, 139.67 (d, J=17.0 Hz), 133.72, 132.95, 132.37, 131.19, 130.93, 129.61, 128.34, 128.05, 127.32, 125.82, 123.41, 99.69, 96.05, 84.61, 80.67, 76.17, 75.66 (d, J=11.3 Hz), 75.35, 75.09, 70.79, 70.21, 70.01, 59.37 (d, J=52.1 Hz), 55.88, 45.63, 44.86, 43.40, 41.46, 41.33, 39.89, 37.27, 37.13, 36.79, 36.51, 36.06, 34.98, 34.26, 31.56, 27.11, 25.46, 22.25, 17.94, 17.68, 17.55, 16.92, 16.31, 15.89, 14.88, 14.28, 13.88, 13.52, 12.82, 9.43, 7.26. dipentanoate CMB ¹H NMR (600 MHz, CDCl₃) δ 6.52 (dd, J=15.1, 10.7 Hz, 1H), 6.33 (s, 1H), 5.83 (d, J=2.0 Hz, 1H), 5.79 (d, J=10.8 Hz, 1H), 5.54 (d, J=9.8 Hz, 1H), 5.48 (dq, J=13.2, 6.4 Hz, 1H), 5.21 (ddd, J=15.3, 8.1, 1.8 Hz, 1H), 5.15 (dd, J=15.0, 9.3 Hz, 1H), 4.92 (dd, J=9.5, 1.6 Hz, 1H), 4.88 (ddd, J=11.9, 9.5, 5.3 Hz, 1H), 4.66 (d, J=4.3 Hz, 1H), 4.60 (d, J=11.0 Hz, 1H), 4.57-4.50 (m, 2H), 4.48 (s, 1H), 3.96 (dt, J=10.5, 3.3 Hz, 1H), 3.90 (dd, J=10.2, 8.0 Hz, 1H), 3.77 (t, J=9.4 Hz, 1H), 3.70 (td, J=10.8, 4.8 Hz, 1H), 3.53-3.46 (m, 1H), 3.49 (s, 3H), 3.33 (dq, J=9.7, 6.2 Hz, 1H), 3.18 (s, 3H), 2.60 (dd, J=11.3, 6.0 Hz, 1H), 2.48 (ddd, J=9.7, 6.9, 2.5 Hz, 1H), 2.30 (t, J=7.6 Hz, 2H), 2.28-2.24 (m, 1H), 2.23 (t, J=7.5 Hz, 2H), 2.15 (ddd, J=12.2, 9.9, 6.3 Hz, 2H), 2.05 (d, J=15.6 Hz, 1H), 2.02-1.92 (m, 1H), 1.92-1.83 (m, 3H), 1.81 (s, 3H), 1.80-1.73 (m, 1H), 1.73-1.66 (m, 1H), 1.65-1.59 (m, OH), 1.58-1.54 (m, 2H), 1.55-1.48 (m, 5H), 1.34-1.26 (m, 2H), 1.23 (dt, J=14.6, 7.4 Hz, 2H), 1.19-1.14 (m, 4H), 1.08-1.01 (m, 1H), 1.00 (d, J=7.1 Hz, 3H), 0.96 (d, J=7.0 Hz, 3H), 0.86 (t, J=7.4 Hz, 3H), 0.81 (dt, J=10.6, 7.6 Hz, 10H), 0.75 (d, J=6.9 Hz, 3H), 0.61 (d, J=7.0 Hz, 3H). ¹³C NMR (151 MHz, CDCl₃) δ 172.49, 165.55, 154.66, 140.87, 140.67, 137.91, 132.68, 131.40, 129.93, 129.77, 126.86, 126.50, 122.68, 98.54, 94.88, 83.20, 80.03, 75.01, 74.42, 73.92, 69.62, 68.97, 68.83, 57.98, 54.63, 44.87, 40.27, 40.17, 38.73, 36.05, 35.95, 35.47, 33.82, 33.18, 33.10, 32.23, 26.18, 25.95, 21.35, 21.09, 20.49, 16.78, 16.39, 15.93, 15.79, 15.02, 13.12, 12.79, 12.72, 12.36, 8.18, 6.11.

### Example 11: Synthesis of 3-pentanoate Concanamycin A (Compound A12)

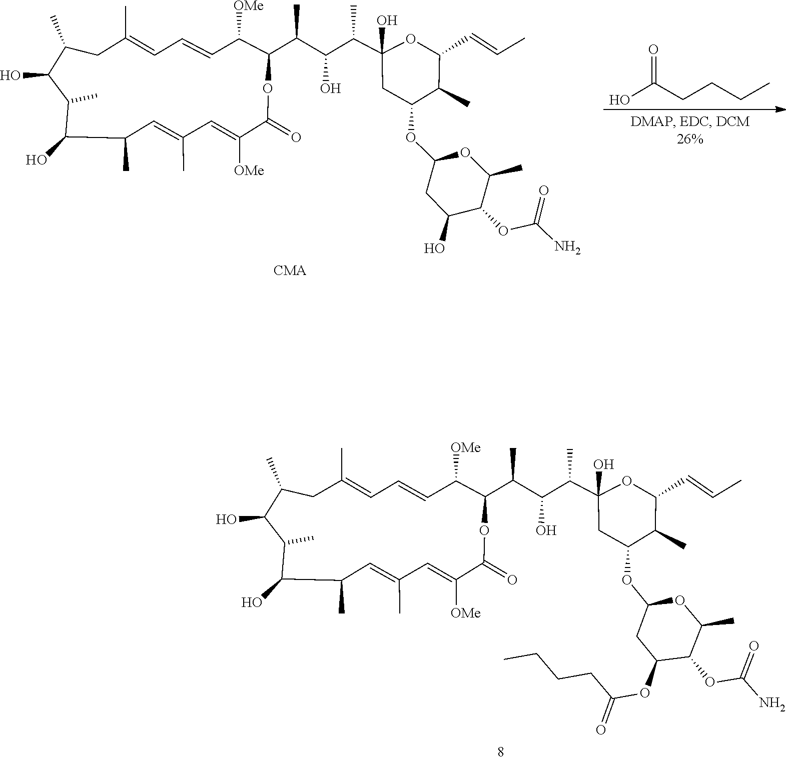

CMA (25 mg, 29 μmol), pentanoic acid (6.3 μL, 58 μmol, 2 eq), DMAP (7.4 mg, 61 μmol, 2.1 eq), EDC (12 mg, 64 μmol, 2.2 eq), DCM (4.09 mL). isolated the product. 3′-pentanoate CMA ¹H NMR (599 MHz, CDCl₃) δ 6.56 (dd, J=15.1, 10.7 Hz, 1H), 6.40 (s, 1H), 5.87 (s, 1H), 5.81-5.75 (m, 1H), 5.68 (d, J=9.8 Hz, 1H), 5.55 (dq, J=13.1, 6.6 Hz, 1H), 5.28 (dd, J=14.9, 8.0 Hz, 1H), 5.22 (dd, J=15.1, 9.0 Hz, 1H), 5.01 (dd, J=9.0, 6.2 Hz, 1H), 5.00-4.91 (m, 1H), 4.73-4.66 (m, 2H), 4.64-4.57 (m, 2H), 4.02 (d, J=10.3 Hz, 1H), 3.97 (t, J=9.2 Hz, 1H), 3.86 (t, J=9.2 Hz, 1H), 3.84-3.81 (m, 1H), 3.77 (td, J=10.8, 4.7 Hz, 1H), 3.56 (s, 3H), 3.44-3.36 (m, 1H), 3.26 (s, 3H), 3.22 (d, J=10.3 Hz, 1H), 2.73 (s, 1H), 2.31 (dt, J=10.7, 7.5 Hz, 4H), 2.22 (dd, J=5.3, 1.9 Hz, 1H), 2.22-2.14 (m, 1H), 2.05-1.91 (m, 5H), 1.87 (s, 3H), 1.75 (s, 1H), 1.75-1.63 (m, 1H), 1.63-1.53 (m, 5H), 1.55-1.44 (m, 1H), 1.32 (p, J=7.5 Hz, 2H), 1.25 (dd, J=11.1, 6.2 Hz, 4H), 1.23-1.10 (m, 3H), 1.07 (td, J=14.0, 12.9, 7.2 Hz, 9H), 0.89 (td, J=7.5, 2.8 Hz, 9H), 0.84 (dd, J=21.0, 7.1 Hz, 3H). ¹³C NMR (151 MHz, CDCl₃) δ 173.52, 166.63, 155.80, 142.11, 141.83, 139.57, 133.29, 132.22, 130.81, 130.64, 127.83, 127.13, 122.99, 99.55, 95.92, 81.29, 79.66, 76.04, 75.55, 75.48, 74.98, 74.29, 70.65, 70.09, 69.86, 59.07, 55.75, 44.72, 43.32, 41.32, 41.20, 39.75, 37.00, 36.85, 36.33, 34.56, 34.13, 26.98, 25.82, 22.76, 22.12, 21.68, 17.80, 17.42, 16.78, 16.39, 14.18, 13.74, 11.66, 9.32, 7.12.

### Example 12: Synthesis of 3′-Acetyl CMA & 3′,9-Diacetyl CMA (Compounds A14 and A15)

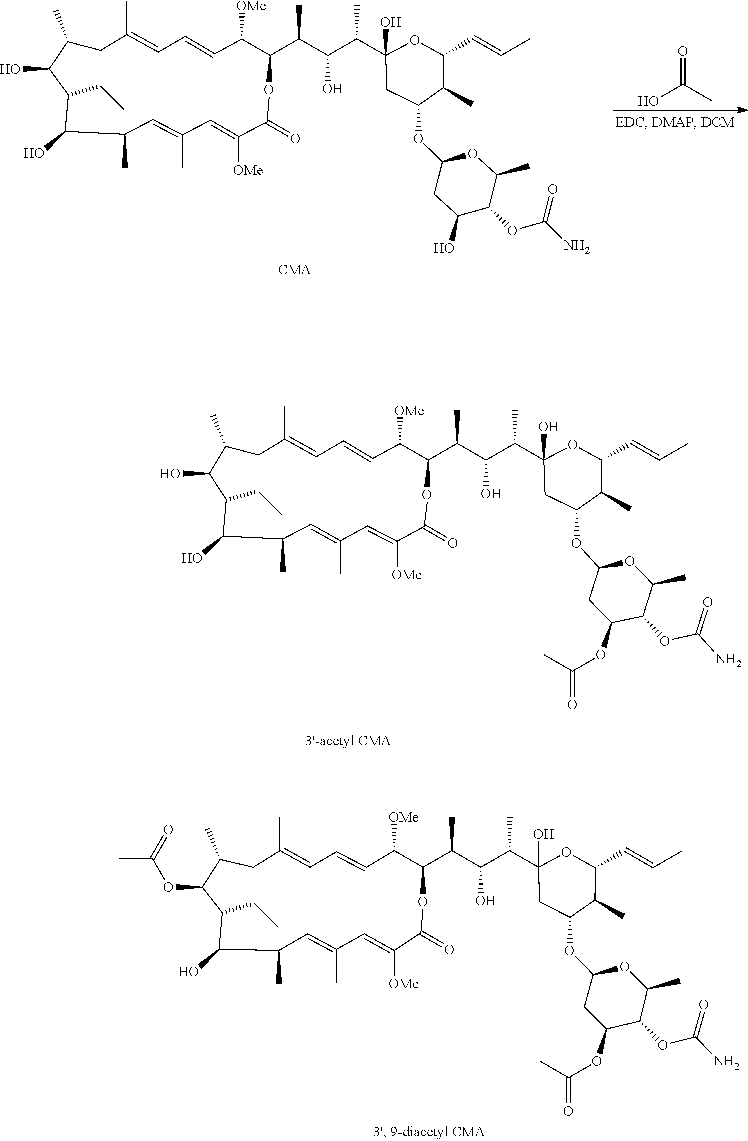

CMA (35 mg, 40 μmol), pentanoic acid (3.5 μL, 61 μmol, 1.5 eq), DMAP (10. mg, 85 μmol, 2.1 eq), EDC (17 mg, 89 μmol, 2.2 eq), DCM (4.9 mL). Acetyl CMA ¹H NMR (600 MHz, CDCl₃) δ 6.57 (dd, J=15.0, 10.6 Hz, 1H), 6.38 (d, J=21.5 Hz, 1H), 5.88 (s, 1H), 5.82-5.75 (m, 1H), 5.68 (d, J=9.9 Hz, 1H), 5.55 (dt, J=13.6, 6.8 Hz, 1H), 5.28 (dd, J=15.3, 8.0 Hz, 1H), 5.21 (dt, J=18.9, 9.4 Hz, 1H), 5.01 (d, J=9.2 Hz, 1H), 4.98-4.91 (m, 1H), 4.70 (d, J=4.2 Hz, 1H), 4.60 (q, J=9.4 Hz, 2H), 4.05-4.00 (m, 1H), 3.97 (t, J=9.2 Hz, 1H), 3.86 (t, J=9.2 Hz, 1H), 3.82 (d, J=10.4 Hz, 1H), 3.77 (td, J=10.8, 4.6 Hz, 1H), 3.56 (s, 3H), 3.40 (dq, J=12.3, 6.3 Hz, 1H), 3.26 (s, 3H), 3.22 (d, J=10.3 Hz, 1H), 2.75 (dt, J=36.6, 8.1 Hz, 1H), 2.32 (dt, J=14.5, 7.2 Hz, 2H), 2.20 (ddd, J=17.5, 11.6, 6.3 Hz, 2H), 2.06 (s, 3H), 2.04-1.93 (m, 5H), 1.87 (s, 3H), 1.74 (dd, J=20.2, 6.1 Hz, 1H), 1.67 (dd, J=22.8, 11.3 Hz, 1H), 1.58 (d, J=6.4 Hz, 3H), 1.51 (dt, J=11.1, 5.8 Hz, 1H), 1.24 (d, J=6.5 Hz, 4H), 1.15 (dd, J=22.7, 8.6 Hz, 3H), 1.07 (td, J=15.1, 13.3, 6.9 Hz, 9H), 0.88 (d, J=6.4 Hz, 3H), 0.88-0.83 (m, 3H), 0.82 (d, J=6.7 Hz, 3H). ¹³C NMR (151 MHz, CDCl₃) δ 170.78, 166.63, 155.80, 142.13, 141.80, 139.61, 133.30, 132.19, 130.79, 130.67, 127.85, 127.08, 122.96, 99.53, 95.91, 81.28, 79.63, 76.04, 75.53, 75.47, 75.01, 74.25, 70.95, 70.07, 69.82, 59.07, 55.75, 44.72, 43.30, 41.30, 41.17, 39.73, 36.92, 36.84, 36.30, 34.56, 22.73, 21.69, 21.15, 17.81, 17.41, 16.78, 16.38, 14.17, 13.39, 11.66, 9.31, 7.12. Diacetyl CMA ¹H NMR (600 MHz, CDCl₃) δ 6.58 (dd, J=15.1, 10.7 Hz, 1H), 6.40 (s, 1H), 5.89 (s, 1H), 5.79 (d, J=10.7 Hz, 1H), 5.63 (d, J=9.8 Hz, 1H), 5.56 (dq, J=13.3, 6.5 Hz, 1H), 5.28 (dd, J=15.3, 8.0 Hz, 1H), 5.22 (dd, J=15.1, 9.2 Hz, 1H), 5.00 (d, J=9.3 Hz, 1H), 4.94 (ddd, J=11.4, 9.4, 5.2 Hz, 1H), 4.85 (d, J=10.9 Hz, 1H), 4.72 (d, J=4.2 Hz, 1H), 4.64-4.56 (m, 2H), 4.05-4.00 (m, 1H), 3.98 (t, J=9.1 Hz, 1H), 3.85 (t, J=9.3 Hz, 1H), 3.77 (td, J=10.8, 4.7 Hz, 1H), 3.68 (dt, J=10.3, 3.5 Hz, 1H), 3.56 (s, 3H), 3.40 (dq, J=12.1, 6.2 Hz, 1H), 3.25 (s, 3H), 2.71-2.61 (m, 2H), 2.33 (dd, J=12.1, 4.8 Hz, 1H), 2.21 (dt, J=10.7, 5.6 Hz, 2H), 2.12 (s, 3H), 2.08 (s, 1H), 2.06 (s, 3H), 2.01 (dd, J=15.6, 6.8 Hz, 1H), 1.95 (s, 3H), 1.88 (s, 3H), 1.76 (q, J=7.3 Hz, 1H), 1.73-1.64 (m, 1H), 1.58 (d, J=6.6 Hz, 4H), 1.28 (dd, J=11.6, 5.4 Hz, 1H), 1.24 (d, J=6.6 Hz, 3H), 1.12 (t, J=11.3 Hz, 1H), 1.07 (d, J=7.2 Hz, 3H), 1.04 (d, J=6.9 Hz, 3H), 0.89 (td, J=10.8, 5.2 Hz, 9H), 0.82 (d, J=6.9 Hz, 3H). ¹³C NMR (151 MHz, CDCl₃) δ 170.99, 170.77, 166.61, 155.74, 141.81, 141.78, 139.13, 133.55, 132.30, 130.79, 130.66, 127.85, 127.39, 123.51, 99.55, 95.92, 81.22, 79.54, 76.05, 75.45, 75.01, 74.59, 70.96, 70.02, 69.82, 59.03, 55.72, 45.27, 43.64, 41.32, 41.19, 39.75, 36.92, 36.62, 34.69, 33.54, 21.30, 21.23, 21.15 (d, J=1.8 Hz), 17.82, 17.41, 16.76, 16.15, 14.14, 13.39, 11.96, 9.25, 7.13.

### Example 13: Synthesis of 3′-penta-2,4-diene CMA (Compound A16)

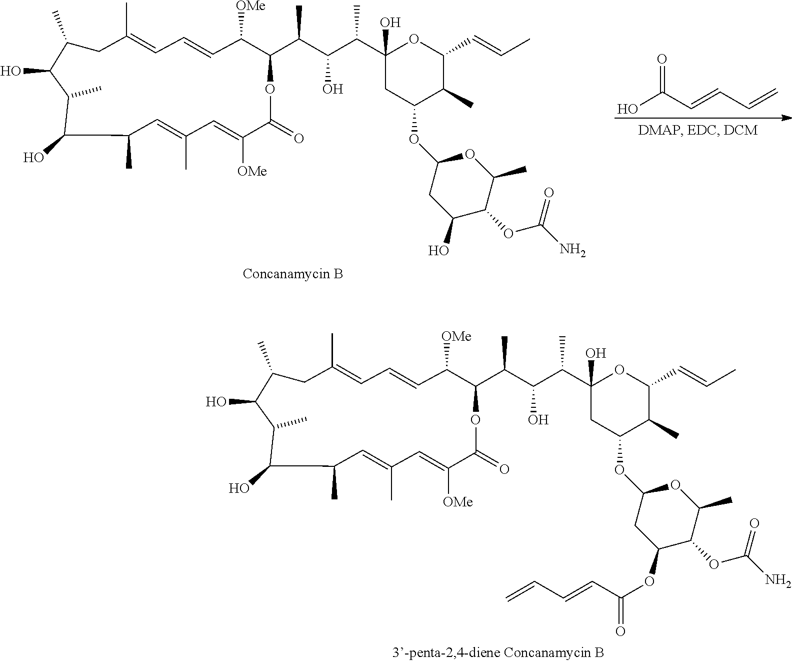

CMA (20 mg, 35 μmol), nonanoic acid (3.5 μL, 35, μmol, 1.5 eq), DMAP (6.0 mg, 49 μmol, 2.1 eq), EDC (9.9 mg, 52 μmol, 2.2 eq), DCM (2.87 mL).*NMR resolution was incredibly pour for the compound. Confirmation of structure was done by both NMR analysis and MS analysis. ¹³C NMR (151 MHz, Acetone) δ 160.03, 149.51, 139.37, 128.35, 124.57, 120.15, 115.29, 93.32, 89.73, 74.28, 71.00, 70.79, 70.57, 69.81, 68.74, 64.77, 63.83, 63.62, 49.52, 35.08, 34.95, 33.52, 23.48, 16.48, 11.59, 11.21, 8.53, 7.95, 7.17, 3.05, −6.23.

### Example 14: Synthesis of 3′,4′-diacetyl CMC & 3′,4′,9-triacetyl CMC (Compounds A17 and A18)

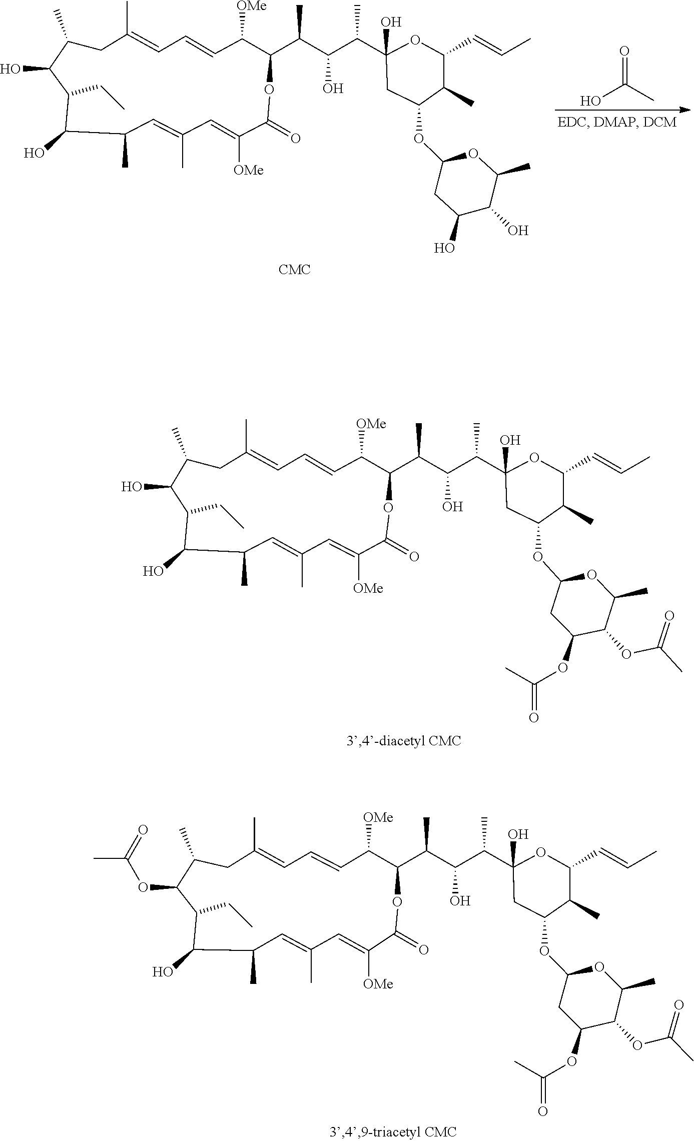

CMC (30 mg, 36 μmol), acetic acid (5.2 μL, 91, μmol, 2.5 eq), DMAP (14 mg, 113 μmol, 3.1 eq), EDC (22.4 mg, 117 μmol, 3.2 eq), DCM (4.5 mL). Diacetyl CMC ¹H NMR (600 MHz, CDCl₃) δ 6.56 (dt, J=20.2, 10.1 Hz, 1H), 6.38 (d, J=21.0 Hz, 1H), 5.88 (s, 1H), 5.83-5.75 (m, 1H), 5.68 (d, J=9.8 Hz, 1H), 5.55 (dq, J=13.3, 6.4 Hz, 1H), 5.28 (dd, J=15.3, 8.0 Hz, 1H), 5.22 (dd, J=15.1, 9.0 Hz, 1H), 5.10-4.98 (m, 1H), 4.93 (ddd, J=11.9, 9.5, 5.3 Hz, 1H), 4.75 (t, J=9.5 Hz, 1H), 4.70 (d, J=4.3 Hz, 1H), 4.62 (dd, J=9.8, 2.1 Hz, 1H), 4.05-3.99 (m, 1H), 3.97 (t, J=9.2 Hz, 1H), 3.86 (t, J=9.3 Hz, 1H), 3.83 (d, J=9.9 Hz, 1H), 3.77 (td, J=10.7, 4.5 Hz, 1H), 3.56 (s, 3H), 3.47-3.39 (m, 1H), 3.26 (s, 3H), 3.22 (d, J=10.3 Hz, 1H), 2.82-2.68 (m, 1H), 2.32 (td, J=12.3, 4.2 Hz, 2H), 2.26-2.20 (m, 1H), 2.19 (dd, J=10.7, 6.6 Hz, 1H), 2.14-2.09 (m, 1H), 2.07 (s, 3H), 2.03 (s, 3H), 1.97 (s, 3H), 1.96 (d, J=5.3 Hz, 1H), 1.85 (d, J=23.5 Hz, 3H), 1.76 (d, J=8.2 Hz, 1H), 1.68 (d, J=34.6 Hz, 1H), 1.58 (d, J=6.4 Hz, 3H), 1.51 (dt, J=11.1, 5.5 Hz, 1H), 1.27 (d, J=7.5 Hz, 1H), 1.19 (d, J=6.2 Hz, 3H), 1.15 (d, J=6.8 Hz, 2H), 1.13 (d, J=10.6 Hz, 1H), 1.07 (td, J=15.5, 13.3, 6.9 Hz, 9H), 0.90-0.80 (m, 9H). ¹³C NMR (151 MHz, CDCl₃) δ 170.61, 170.29, 166.63, 142.12, 141.81, 139.57, 133.29, 132.21, 130.79, 130.66, 127.86, 127.10, 122.97, 99.54, 95.88, 81.28, 79.63, 76.04, 75.54, 75.45, 74.28, 73.99, 71.03, 70.07, 69.62, 59.07, 55.75, 44.70, 43.30, 41.30, 41.17, 39.71, 36.87, 36.32, 34.55, 22.74, 21.67, 21.09, 20.96, 17.81, 17.47, 16.77, 16.38, 14.17, 13.39, 11.66, 9.31, 7.13. ¹³C NMR (151 MHz, CDCl₃) δ 170.61, 170.29, 166.63, 142.12, 141.81, 139.57, 133.29, 132.21, 130.79, 130.66, 127.86, 127.10, 122.97, 99.54, 95.88, 81.28, 79.63, 76.04, 75.54, 75.45, 74.28, 73.99, 71.03, 70.07, 69.62, 59.07, 55.75, 44.70, 43.30, 41.30, 41.17, 39.71, 36.87, 36.32, 34.55, 22.74, 21.67, 21.09, 20.96, 17.81, 17.47, 16.77, 16.38, 14.17, 13.39, 11.66, 9.31, 7.13. Triacetyl CMC ¹H NMR (600 MHz, CDCl₃) δ 6.58 (dd, J=15.0, 10.7 Hz, 1H), 6.40 (s, 1H), 5.89 (d, J=2.1 Hz, 1H), 5.79 (d, J=10.7 Hz, 1H), 5.63 (d, J=9.8 Hz, 1H), 5.56 (dq, J=13.2, 6.5 Hz, 1H), 5.28 (ddd, J=15.2, 8.0, 1.8 Hz, 1H), 5.22 (dd, J=15.0, 9.2 Hz, 1H), 5.00 (dd, J=9.3, 1.6 Hz, 1H), 4.93 (ddd, J=11.9, 9.5, 5.3 Hz, 1H), 4.85 (d, J=11.0 Hz, 1H), 4.75 (t, J=9.5 Hz, 1H), 4.72 (d, J=4.2 Hz, 1H), 4.62 (dd, J=9.8, 1.9 Hz, 1H), 4.02 (ddd, J=10.9, 4.4, 2.2 Hz, 1H), 3.98 (dd, J=10.3, 8.0 Hz, 1H), 3.85 (t, J=9.3 Hz, 1H), 3.78 (td, J=10.7, 4.7 Hz, 1H), 3.68 (ddt, J=7.3, 4.5, 2.6 Hz, 1H), 3.56 (s, 3H), 3.42 (dq, J=9.9, 6.1 Hz, 1H), 3.25 (s, 3H), 2.73-2.62 (m, 2H), 2.33 (dd, J=12.1, 4.8 Hz, 1H), 2.22 (tdd, J=15.5, 6.8, 3.5 Hz, 2H), 2.12 (s, 3H), 2.10 (s, 1H), 2.07 (s, 3H), 2.03 (s, 3H), 2.02-1.97 (m, 1H), 1.96-1.94 (m, 3H), 1.88 (s, 3H), 1.79-1.71 (m, 1H), 1.71-1.65 (m, 1H), 1.58 (dd, J=6.6, 1.6 Hz, 4H), 1.31-1.25 (m, 1H), 1.19 (d, J=6.2 Hz, 3H), 1.15-1.09 (m, 2H), 1.07 (d, J=7.2 Hz, 3H), 1.04 (d, J=7.0 Hz, 3H), 0.88 (td, J=10.8, 10.0, 5.1 Hz, 10H), 0.82 (d, J=6.9 Hz, 3H). ¹³C NMR (151 MHz, CDCl₃) δ 170.98, 170.61, 170.29, 166.61, 141.80, 141.78, 139.12, 133.54, 132.30, 130.79, 130.66, 127.86, 127.39, 123.51, 99.55, 95.88, 81.22, 79.54, 76.05, 75.44, 74.60, 73.99, 71.03, 70.02, 69.62, 59.03, 55.72, 45.27, 43.64, 41.32, 41.18, 39.73, 36.88, 36.62, 34.69, 33.54, 21.30, 21.23, 21.16, 21.09, 20.96, 17.82, 17.47, 16.76, 16.15, 14.14, 13.40, 11.97, 9.25, 7.13.

### Example 15: Synthesis of 23-acetyl CMF & 3′,9-diacteyl CMF (Compounds A19 and A20)

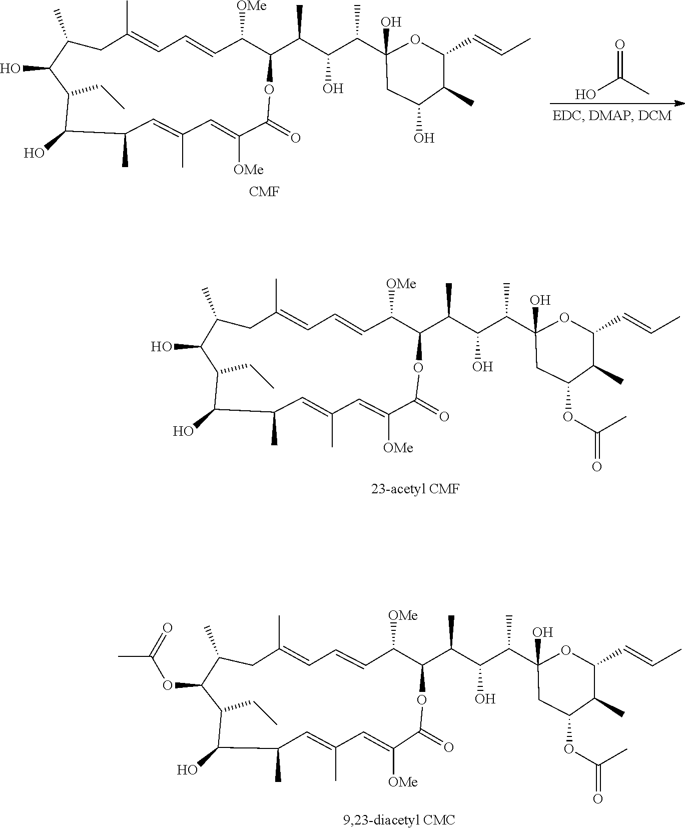

CMF (20 mg, 29 μmol), acetic acid (2.5 μL, 43, μmol, 1.5 eq), DMAP (7.4 mg, 61 μmol, 2.1 eq), EDC (12 mg, 64 μmol, 2.2 eq), DCM (4.1 mL). Isolated 23-acetyl CMF and 9,23-diacetyl CMF. Acetyl CMF ¹H NMR (600 MHz, CDCl₃) δ 6.56 (dd, J=15.2, 10.9 Hz, 1H), 6.37 (d, J=20.5 Hz, 1H), 5.81 (s, 1H), 5.80-5.72 (m, 1H), 5.68 (d, J=9.7 Hz, 1H), 5.58 (dd, J=14.3, 7.0 Hz, 1H), 5.28 (dd, J=15.4, 8.2 Hz, 1H), 5.22 (dd, J=15.1, 9.1 Hz, 1H), 5.04-4.95 (m, 2H), 4.64 (d, J=4.4 Hz, 1H), 4.07 (t, J=9.1 Hz, 1H), 4.00 (d, J=11.5 Hz, 1H), 3.84 (q, J=10.2, 9.7 Hz, 2H), 3.56 (d, J=2.2 Hz, 3H), 3.25 (d, J=2.2 Hz, 3H), 3.22 (d, J=10.6 Hz, 1H), 2.72 (t, J=8.2 Hz, 1H), 2.37 (dd, J=12.0, 4.8 Hz, 1H), 2.30 (s, 1H), 2.20-2.14 (m, 1H), 2.05 (d, J=2.2 Hz, 3H), 2.04-1.93 (m, 5H), 1.87 (s, 3H), 1.74 (q, J=7.0, 5.4 Hz, 1H), 1.59 (d, J=6.4 Hz, 3H), 1.53-1.48 (m, 1H), 1.40 (d, J=8.0 Hz, 1H), 1.23-1.20 (m, 1H), 1.15 (td, J=15.7, 7.7 Hz, 2H), 1.09 (d, J=6.6 Hz, 3H), 1.04 (t, J=7.9 Hz, 6H), 0.86 (q, J=7.9, 7.4 Hz, 3H), 0.80 (t, J=8.4 Hz, 6H). ¹³C NMR (151 MHz, CDCl₃) δ 170.53, 166.55, 142.07, 141.86, 139.50, 133.25, 132.23, 130.52, 130.46, 128.29, 127.14, 122.99, 99.42, 81.28, 79.62, 75.54, 75.01, 74.30, 73.21, 70.00, 59.04, 55.73, 44.68, 43.31, 41.23, 40.66, 39.81, 36.83, 36.35, 34.54, 22.75, 21.65, 21.33, 17.81, 16.79, 16.40, 14.19, 13.30, 11.66, 9.33, 7.07. Diacetyl CMF ¹H NMR (600 MHz, CDCl₃) δ 6.58 (dd, J=15.1, 10.7 Hz, 1H), 6.39 (s, 1H), 5.82 (d, J=2.0 Hz, 1H), 5.79 (d, J=10.7 Hz, 1H), 5.63 (d, J=9.9 Hz, 1H), 5.63-5.54 (m, 1H), 5.29 (ddd, J=15.4, 8.2, 1.8 Hz, 1H), 5.22 (dd, J=15.0, 9.2 Hz, 1H), 4.99 (td, J=10.6, 3.3 Hz, 2H), 4.85 (d, J=10.9 Hz, 1H), 4.67 (d, J=4.3 Hz, 1H), 4.08 (dd, J=10.3, 8.1 Hz, 1H), 4.01 (ddd, J=10.9, 4.4, 2.2 Hz, 1H), 3.84 (t, J=9.3 Hz, 1H), 3.70-3.63 (m, 1H), 3.56 (s, 3H), 3.24 (s, 3H), 2.72-2.61 (m, 2H), 2.37 (dd, J=11.8, 4.8 Hz, 1H), 2.23-2.14 (m, 1H), 2.12 (s, 3H), 2.09 (d, J=15.3 Hz, 1H), 2.06 (s, 3H), 2.01 (dd, J=15.5, 6.7 Hz, 1H), 1.95 (s, 3H), 1.88 (s, 3H), 1.77-1.71 (m, 1H), 1.60 (dd, J=6.5, 1.6 Hz, 3H), 1.59-1.54 (m, 1H), 1.40 (tdd, J=13.0, 8.4, 5.3 Hz, 1H), 1.24-1.20 (m, 1H), 1.10-1.07 (m, 1H), 1.04 (dd, J=7.1, 5.5 Hz, 6H), 0.90 (d, J=6.9 Hz, 3H), 0.87 (d, J=3.5 Hz, 4H), 0.80 (t, J=6.6 Hz, 6H). ¹³C NMR (151 MHz, CDCl₃) δ 170.98, 170.54, 166.54, 141.86, 141.74, 139.06, 133.52, 132.32, 130.54, 130.46, 128.30, 127.42, 123.52, 99.44, 81.23, 79.54, 75.44, 75.00, 74.62, 73.22, 69.96, 59.01, 55.70, 45.25, 43.65, 41.25, 40.68, 39.83, 36.61, 34.69, 33.57, 21.33, 21.30, 21.25, 21.16, 17.82, 16.77, 16.16, 14.15, 13.31, 11.97, 9.27, 7.08.

### Example 16: Synthesis of DLP CMA & DLP, DLP CMA

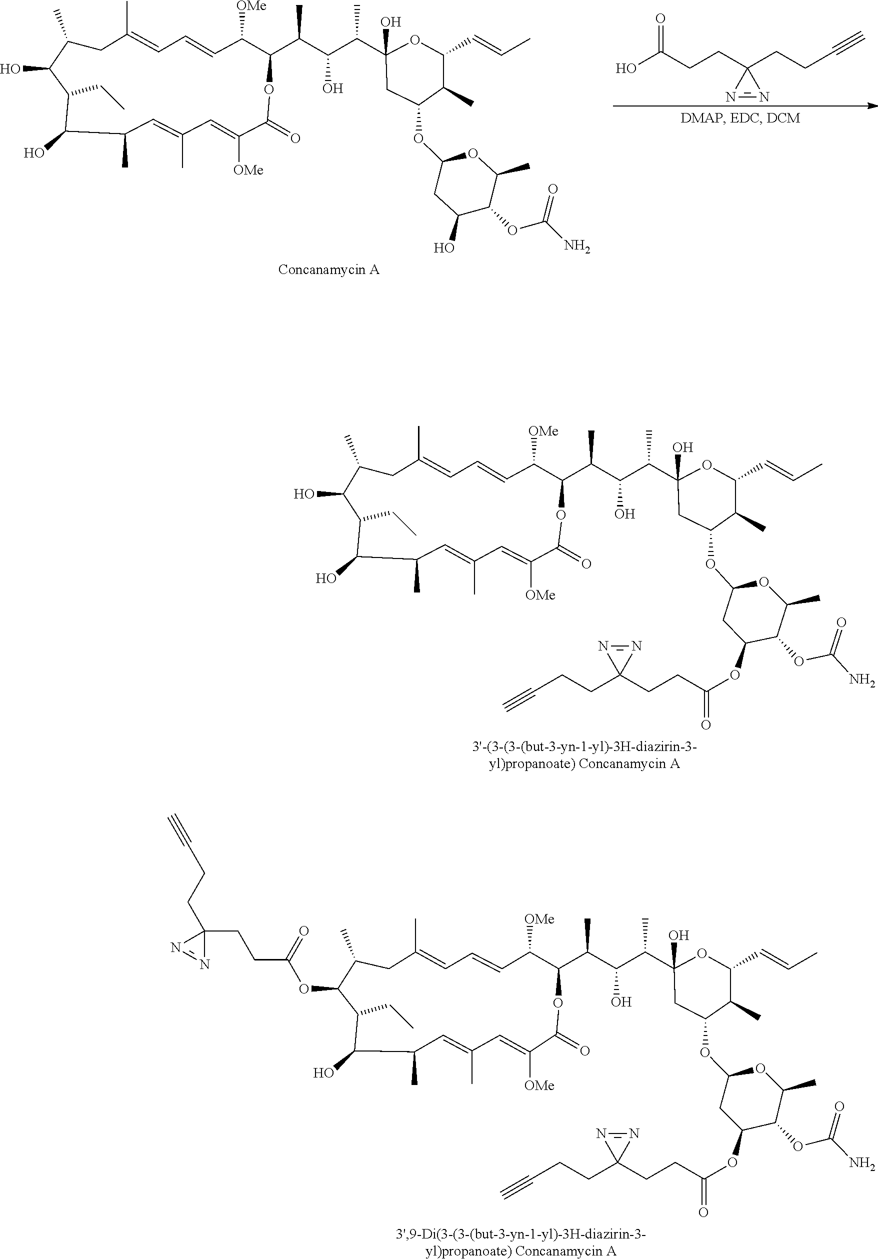

CMA (30 mg, 34 μmol), 3-(3-(but-3-yn-1-yl)-3H-diazirin-3-yl)propanoic acid (8.5 μL, 51 μmol, 1.5 eq), DMAP (8.7 mg, 72 μmol, 2.1 eq), EDC (14 mg, 75 μmol, 2.2 eq), DCM (4.8 mL). DLP CMA ¹H NMR (600 MHz, CDCl₃) δ 6.55 (td, J=22.1, 18.1, 10.9 Hz, 1H), 6.43-6.35 (m, 1H), 5.86 (d, J=17.9 Hz, 1H), 5.83-5.75 (m, 1H), 5.68 (d, J=9.8 Hz, 1H), 5.55 (dd, J=14.5, 6.8 Hz, 1H), 5.28 (dd, J=15.3, 8.0 Hz, 1H), 5.22 (dd, J=15.3, 9.2 Hz, 1H), 5.01 (d, J=9.1 Hz, 1H), 4.95 (tq, J=9.4, 3.7, 2.2 Hz, 1H), 4.70 (d, J=4.3 Hz, 1H), 4.64-4.55 (m, 2H), 4.05-3.98 (m, 1H), 3.97 (d, J=9.3 Hz, 1H), 3.86 (dt, J=9.3, 4.6 Hz, 1H), 3.84-3.81 (m, 1H), 3.81-3.73 (m, 1H), 3.56 (d, J=2.2 Hz, 3H), 3.43-3.34 (m, 1H), 3.26 (d, J=2.2 Hz, 3H), 3.22 (d, J=10.3 Hz, 1H), 2.82-2.69 (m, 1H), 2.32 (ddd, J=12.5, 5.2, 2.6 Hz, 2H), 2.26-2.19 (m, 1H), 2.19 (t, J=8.8 Hz, 1H), 2.13 (td, J=7.0, 6.2, 4.0 Hz, 2H), 2.06-1.99 (m, 4H), 1.97 (s, 4H), 1.88 (s, 3H), 1.86-1.75 (m, 2H), 1.78-1.73 (m, 1H), 1.73-1.65 (m, 1H), 1.68-1.63 (m, 2H), 1.59 (d, J=6.5 Hz, 3H), 1.54-1.48 (m, 1H), 1.24 (td, J=7.7, 6.4, 3.7 Hz, 4H), 1.19-1.11 (m, 3H), 1.12-1.02 (m, 9H), 0.91-0.80 (m, 9H). ¹³C NMR (151 MHz, CDCl₃) δ 171.75, 166.63, 155.72, 142.14, 141.79, 139.62, 133.31, 132.19, 130.78, 130.68, 127.85, 127.08, 122.96, 99.53, 95.87, 82.61, 81.27, 79.62, 76.08, 75.53, 75.47, 74.88, 74.25, 71.31, 70.07, 69.80, 69.33, 59.06, 55.75, 44.72, 43.30, 41.29, 41.17, 39.73, 36.88, 36.84, 36.30, 34.56, 31.93, 28.54, 27.72, 27.59, 22.73, 21.69, 17.81, 17.40, 16.78, 16.38, 14.17, 13.39, 13.23, 11.67, 9.31, 7.12.

DLP, DLP CMA ¹H NMR (600 MHz, CDCl₃) δ 6.59 (dd, J=15.0, 10.8 Hz, 1H), 6.39 (s, 1H), 5.89 (d, J=2.1 Hz, 1H), 5.78 (d, J=10.7 Hz, 1H), 5.62 (d, J=9.8 Hz, 1H), 5.56 (dq, J=13.2, 6.5 Hz, 1H), 5.28 (ddd, J=15.3, 8.1, 1.8 Hz, 1H), 5.22 (dd, J=15.0, 9.2 Hz, 1H), 5.00 (dd, J=9.3, 1.6 Hz, 1H), 4.95 (ddd, J=11.9, 9.5, 5.2 Hz, 1H), 4.85 (d, J=11.0 Hz, 1H), 4.72 (d, J=4.3 Hz, 1H), 4.64-4.56 (m, 2H), 4.02 (ddd, J=10.8, 4.4, 2.2 Hz, 1H), 3.98 (dd, J=10.2, 8.0 Hz, 1H), 3.85 (t, J=9.3 Hz, 1H), 3.77 (td, J=10.7, 4.7 Hz, 1H), 3.64 (ddd, J=10.3, 4.5, 2.8 Hz, 1H), 3.56 (s, 3H), 3.40 (dq, J=9.5, 6.2 Hz, 1H), 3.25 (s, 3H), 2.67 (ddd, J=16.7, 8.0, 4.6 Hz, 2H), 2.32 (dd, J=12.1, 4.7 Hz, 1H), 2.26-2.18 (m, 2H), 2.13 (dd, J=8.0, 6.6 Hz, 3H), 2.11-2.07 (m, 1H), 2.04 (dd, J=7.4, 2.7 Hz, 1H), 2.02 (d, J=4.9 Hz, 6H), 2.01-1.97 (m, 1H), 1.95 (d, J=1.3 Hz, 3H), 1.88 (s, 3H), 1.86 (dd, J=7.6, 1.5 Hz, 1H), 1.77-1.73 (m, 1H), 1.71-1.68 (m, 4H), 1.58 (dd, J=6.4, 1.6 Hz, 3H), 1.56 (d, J=7.8 Hz, 1H), 1.27 (s, 1H), 1.24 (d, J=6.2 Hz, 3H), 1.12 (td, J=10.5, 9.3, 2.6 Hz, 1H), 1.11-1.07 (m, 1H), 1.07 (d, J=7.2 Hz, 3H), 1.04 (d, J=6.9 Hz, 3H), 0.93-0.84 (m, 10H), 0.82 (d, J=6.9 Hz, 3H). ¹³C NMR (151 MHz, CDCl₃) δ 172.06, 171.75, 166.62, 155.67, 141.80, 141.75, 139.08, 133.58, 132.30, 130.79, 130.66, 127.86, 127.42, 123.54, 99.55, 95.87, 82.61, 82.57, 81.21, 79.98, 76.08, 75.45, 75.43, 74.88, 74.49, 71.32, 70.01, 69.80, 69.33, 69.28, 59.02, 55.72, 45.34, 43.70, 41.32, 41.19, 39.75, 36.88, 36.58, 34.73, 33.38, 32.20, 31.94, 28.54, 28.26, 27.73, 27.70, 27.64, 27.59, 21.38, 21.11, 17.82, 17.40, 16.75, 16.12, 14.14, 13.39, 13.28, 13.23, 12.02, 9.24, 7.12.

### Example 17: Synthesis of DLP CMB (Compound A21)

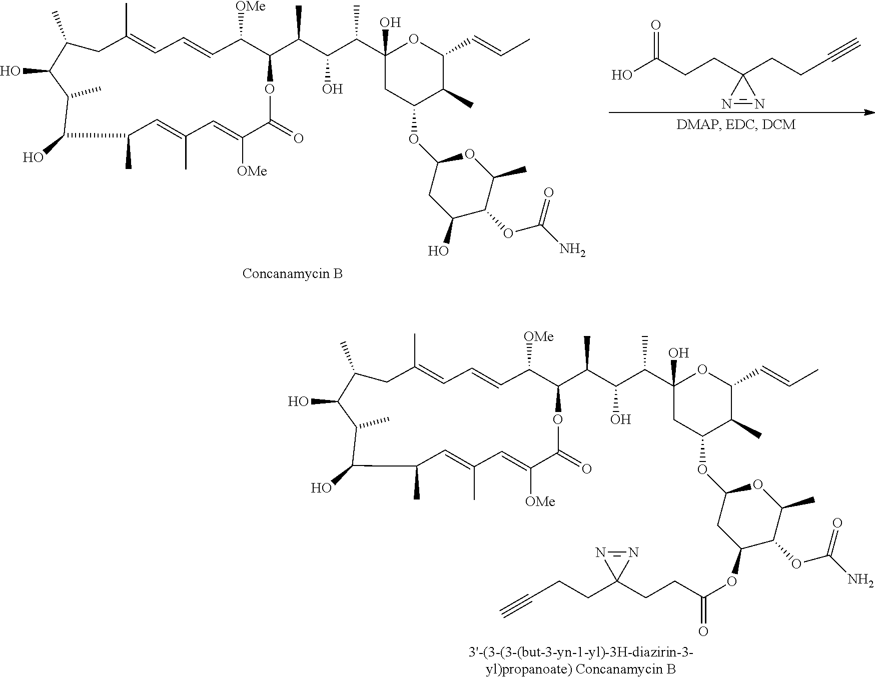

¹H NMR (599 MHz, CDCl₃, 323 K) δ 6.53 (dd, J=15.1, 10.6 Hz, 1H), 6.37 (s, 1H), 5.80 (d, J=10.6 Hz, 1H), 5.65 (s, 1H), 5.55 (dq, J=13.1, 6.4 Hz, 1H), 5.34-5.27 (m, 1H), 5.23 (dd, J=15.2, 8.9 Hz, 1H), 5.03 (d, J=9.1 Hz, 1H), 4.96 (ddd, J=12.1, 9.3, 5.2 Hz, 1H), 4.63-4.53 (m, 4H), 4.07-4.01 (m, 1H), 3.97 (dd, J=10.3, 7.6 Hz, 1H), 3.85 (q, J=9.4 Hz, 2H), 3.77 (td, J=10.7, 4.6 Hz, 1H), 3.57 (s, 2H), 3.40 (dq, J=12.4, 6.3 Hz, 1H), 3.28 (s, 1H), 3.25 (s, 3H), 2.76-2.69 (m, 1H), 2.30 (dd, J=12.0, 4.8 Hz, 1H), 2.20 (dt, J=15.4, 7.3 Hz, 2H), 2.12 (t, J=7.6 Hz, 2H), 2.00 (dt, J=17.1, 4.8 Hz, 8H), 1.82 (d, J=10.8 Hz, 3H), 1.82-1.73 (m, 2H), 1.76-1.71 (m, 1H), 1.73-1.65 (m, 1H), 1.63 (t, J=7.4 Hz, 2H), 1.59 (d, J=6.5 Hz, 3H), 1.38-1.16 (m, 5H), 1.09 (s, OH), 1.06 (t, J=7.1 Hz, 7H), 0.88 (t, J=7.9 Hz, 6H), 0.82 (d, J=6.9 Hz, 3H).

### Example 18: Synthesis of DLP CMC (Compound A22)

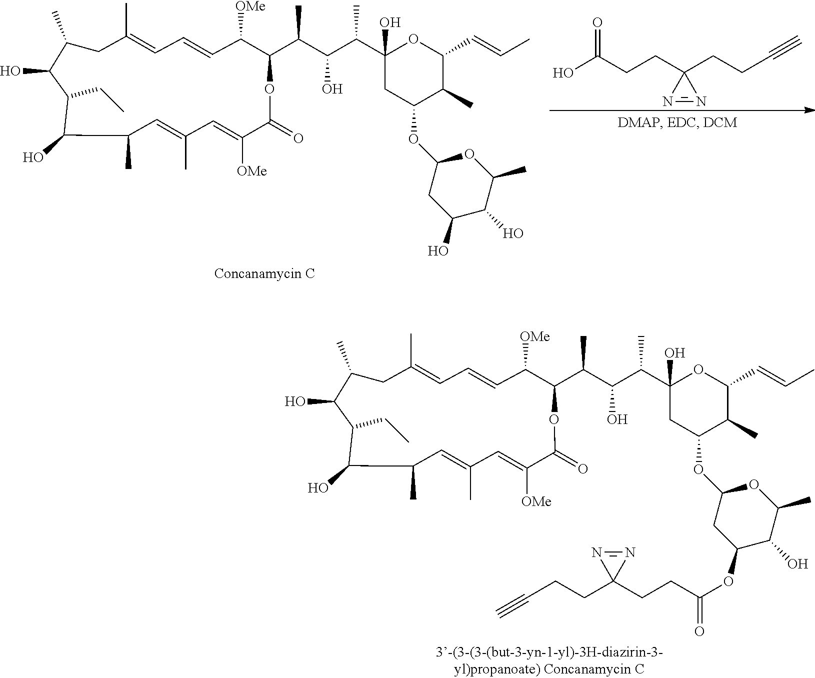 [SMILES: C/C=C/[C@H]1O[C@@](O)([C@@H](C)[C@H](O)[C@H](C)[C@H]2OC(=O)/C(OC)=C/C(C)=C/[C@@H](C)[C@@H](O)[C@@H](CC)[C@@H](O)[C@H](C)C/C(C)=C/C=C/[C@@H]2OC)C[C@@H](O[C@@H]2C[C@H](O)[C@@H](O)[C@H](C)O2)[C@@H]1C ; C#CCCC1(CCC(=O)O)N=N1 ; C#CCCC1(CCC(=O)O[C@H]2C[C@@H](O[C@@H]3C[C@](O)([C@@H](C)[C@H](O)[C@H](C)[C@H]4OC(=O)/C(OC)=C/C(C)=C/[C@@H](C)[C@@H](O)[C@@H](CC)[C@@H](O)[C@H](C)C/C(C)=C/C=C/[C@@H]4OC)O[C@H](/C=C/C)[C@H]3C)O[C@@H](C)[C@@H]2O)N=N1]

¹H NMR (599 MHz, CDCl₃) δ 6.58-6.51 (m, 1H), 6.38 (s, 1H), 5.76 (s, 1H), 5.68 (s, 1H), 5.56 (tt, J=13.0, 7.2 Hz, 1H), 5.33-5.26 (m, 1H), 5.22 (dd, J=15.4, 9.2 Hz, 1H), 5.02 (d, J=8.9 Hz, 1H), 4.81 (ddd, J=12.7, 8.1, 5.2 Hz, 1H), 4.64-4.59 (m, 2H), 4.03 (s, 2H), 4.01-3.95 (m, 1H), 3.91-3.83 (m, 1H), 3.82 (s, 2H), 3.78 (td, J=10.7, 4.8 Hz, 1H), 3.56 (d, J=1.1 Hz, 3H), 3.50-3.45 (m, 1H), 3.31 (ddt, J=11.0, 8.2, 3.9 Hz, 2H), 3.29-3.24 (m, 3H), 2.73 (s, 1H), 2.63-2.46 (m, 4H), 2.35-2.29 (m, 1H), 2.31 (s, 2H), 2.24-2.15 (m, 2H), 2.00 (td, J=2.5, 1.0 Hz, 1H), 1.98 (s, 5H), 1.96-1.91 (m, 3H), 1.86 (s, 2H), 1.78-1.59 (m, 5H), 1.40-1.32 (m, 3H), 1.34-1.30 (m, 3H), 1.25 (h, J=6.5, 5.9 Hz, 3H), 1.18 (dt, J=12.1, 4.8 Hz, 1H), 1.17-1.09 (m, 2H), 1.10-1.01 (m, 7H), 0.96-0.84 (m, 3H), 0.86 (s, 5H), 0.82 (d, J=6.9 Hz, 2H).

### Example 19: Cell Based Assay

### Preparation of Primary CD4 T Lymphocytes

Anonymized leukocytes isolated by apheresis were obtained from New York Blood Center and peripheral blood mononuclear cells (PBMCs) were isolated by Ficoll-Paque Plus (17144002; GE Healthcare) centrifugation using SepMate tubes (85450; StemCell Technologies) according to the manufacturers protocol. CD8⁺ lymphocytes were depleted with Dynabeads according to the manufacturer's protocol (11147D; Invitrogen) and the remaining cells were incubated at a density of 1×10⁶ cells per milliliter in 20 milliliters of R10 medium and stimulated with 10 ug/mL phytohemagglutinin-L (PHA-L) (431784; Millipore Sigma) in 70 mL T25 cell culture flasks. Then, 20-24 hours post-PHA activation, 15 mL of R10/PHA medium is removed and replaced with 5 mL of R10+100u/mL Recombinant Human IL-2 (202|L010; R&D Systems). 48 hours after IL-2 stimulation, primary CD4⁺ T cells were infected via spinoculation with previously described HIV ΔGPE construct.

### Viral Construct & Infections

Infectious supernatants for the HIV ΔGPE construct were prepared by cotransfection of HEK-293T cells using pelyethyenimine (PEI) as previously described [2] with the ΔGPE construct, the HIV packaging plasmid pCMV-HIV and the broad tropism envelope glycoprotein G of Vesicular Stomatitis Virus (VSV-G) at a mass ratio of 1:1:1. Infections were performed by spinoculation. For spinoculation, cells are plated at a density of 1×10⁶ cells/well in a 24 well plate and spun for 2 hours at 1050×g in 1 mL of viral supernatant+4 ug/mL hexadimethrine bromide (polybrene, H9268, Sigma-Aldrich). After spinoculation, cells are resuspended in 2 mL R10+50u/mL IL-2 per well.

### Compound Preparation

All compounds were transferred to pre-weighed vials (weighed in triplicate) for dilution calculations prior to being dissolved in sterile DMSO (Sigma-Aldrich Cat #: 276885). 5 mM or 10 mM solutions were made depending on the overall mass of the material and serial dilutions (in DMSO) were completed to achieve a 100 μM solution. Serial dilutions were vortexed after each step. 50 μL of the 100 μM solution was transferred to a glass vial insert (will add brand later) and stored at −20° C., if necessary, before testing. Thermo Scientific™ E1-ClipTip Bluetooth electronic single channel pipettes were used to throughout the entire sample preparation to avoid carry over of errors due to miscalibration or pipette tips variation.

### Compound Treatments of Primary CD4+ T Cells

48 hours after spinoculation, cells were pooled and plated in flat bottom 96-well plate. Mock cells were plated at 1×10⁵ cells/well in 100 μL R10+100 u/mL IL-2 while HIV ΔGPE infected cells were plated at 1×10⁵ cells/well in 50 μL R10+100 u/mL IL-2. Compounds were solubilized in sterile DMSO (Sigma-Aldrich Cat #: 276885) to a concentration of 100 μM and frozen at −20° C. for storage. Compounds were thawed at room temperature and diluted in R10 medium to 2× starting concentration. Compounds were then serially diluted in a 96-well round bottom plate in R10. Dilutions of compounds were then added to respective ΔGPE and Mock plates to bring final concentration of compounds to 1× and IL-2 concentration to 50u/mL.

### Flow Cytometry Staining

All ΔGPE infected cells were collected 24 hours after treatment. Cells were suspended in Lysotracker Red DND-99 (L7528; Invitrogen, 1:5000) and HLA-A2 (BB7.2 from HB-82 hybridoma as previously described [3], 0.5 μg/mL) in phosphate buffered saline (PBS) for 1 hr at 37°. The cells were washed once with FACS buffer (2% fetal bovine serum (26140079, Gibco), 1% human antibody serum (BP2525, Fisher), 2 mM HEPES (151630080, Gibco) 0.025% sodium azide (S8032, Sigma-Aldrich) in PBS). After washing, the cells were resuspended in 4 ng/mL DAPI (4′,6-diamidino-2-phenylindole, 62248, Thermo Scientific) for viability and 1:1000 goat anti-mouse IgG2b-Alexa Fluor 647 (A21242, Invitrogen) in FACS buffer for 5 minutes at room temperature and after, washed with FACS buffer, then finally fixed in 2% paraformaldehyde for 30 minutes at room temperature.

All Mock cells were collected 72 hours after treatment. Cells were suspended in 4 ng/mL DAPI in FACS buffer for 20 minutes on ice, washed once with FACS buffer and fixed in 2% paraformaldehyde for 30 minutes at room temperature.

In all experiments, flow cytometry data was collected with a BioRad Ze5 cytometer and data was analyzed using FlowJo software. Cells were gated sequentially by forward scatter vs. side scatter for cells, doublet exclusion (forward scatter area vs. height), and exclusion of DAPI for viable cells.

### Calculation and Statistical Analysis:

$$MFI = median fluorescence Intensity of MHC - I \\ Fold downmodulation = {MFI}_{uninfected} / {MFI}_{infected} \\ Normalized Nef activity = Fold downmodulation MHC - {I}_{sample} / Fold downmodulation MHC - {I}_{solvent}$$

All statistical analyses were performed using GraphPad Prism software. Curves were generated using GraphPad Prism software using [Inhibitor] vs. response with variable slope (four parameters).

The results of the anti-Nef activity, toxicity, and lysosomal acidification assays are summarized in Table B, below.

TABLE B

| Name | No. | Nef | Lysotracker | Viability | Viability:Nef | Lys:Nef |
|---|---|---|---|---|---|---|
| CMA | 1 | 0.18 | 1.7 | 1.3 | 6.6 | 9.4 |
| Bafilomycin A₁ | 2 | 12 | 35 | 16 | 1.4 | 2.3 |
| Archazolid A | 3 | 0.47 | 1.6 | .74 | 1.6 | 3.6 |
| Ring Open | S8 | 310 | >1000 | 520 | 1.7 | — |
| Bafilomycin A₁ | | | | | | |
| 21-acetyl Bafilomycin | S9 | 4.5 | 23 | 10. | 2.4 | 5.1 |
| A₁ | | | | | | |
| 21-pentanoate | S10 | 37 | 57 | 51 | 1.4 | 1.5 |
| Bafilomycin A₁ | | | | | | |
| 21-nonanoate | S11 | 130 | 150 | 140 | 1.1 | 1.2 |
| Bafilomycin A₁ | | | | | | |
| 21-penta-2,4-dienoate | S12 | 17 | 24 | 22 | 1.4 | 1.5 |
| Bafilomycin A₁ | | | | | | |
| 21- | S13 | 92 | 140 | 130 | 1.4 | 1.5 |
| benzoateBafilomycin | | | | | | |
| A₁ | | | | | | |
| Leucanicidin | S14 | 1.2 | 2.5 | 1.4 | 1.3 | 2.1 |
| PC-766B | S15 | 1.2 | 2.5 | 1.3 | 1.1 | 2.1 |
| Elaiophylin | S16 | 250 | ND | 160 | N/A | 0.55 |
| 7-one Bafilomycin A₁ | S17 | >1000 | >1000 | >1000 | — | — |
| 7,21-one Bafilomycin | S18 | >1000 | >1000 | >1000 | — | — |
| A₁ | | | | | | |
| CMF | 6 | 1.6 | 5.8 | 2.9 | 1.8 | 4.0 |
| CMX | 7 | 9.1 | 38 | 14 | 1.6 | 4.1 |
| CMC | 5 | 0.71 | 3.7 | 1.3 | 2.3 | 7.1 |
| 21-Deoxy-16-Hydroxy | S25 | 400 | >1000 | >1000 | — | — |
| CMF | | | | | | |
| CMB | 4 | 1.2 | 26 | 7.0 | 5.5 | 24 |
| 3′-Pentanoate CMA | 8 | 0.75 | 11 | 3.5 | 4.8 | 15 |
| 3′-Pentanoate CMB | 9 | 3.4 | 110 | 27 | 7.9 | 32 |
| 3′,9-dipentanoate CMB | 10 | 6.3 | 130 | 35 | 5.8 | 21 |
| 3′-nonanoate CMB | 11 | 9.1 | 170 | 36 | 4.3 | 20. |
| 3′-Acetyl CMB | 12 | 0.66 | 20 | 5.3 | 8.0 | 21 |
| 3′,9-diacetyl CMB | 13 | 0.99 | 38 | 8.0 | 8.8 | 42 |
| 3′-Acetyl CMA | 14 | 0.20 | 3.7 | 1.6 | 8.5 | 19 |
| 3′,9-diacetyl CMA | 15 | 0.39 | 7.9 | 3.9 | 11 | 21 |
| 10-deoxymethynolide | S29 | 350.00 | 1720.00 | 140.00 | 0.39 | 4.8 |
| YC-17 | S30 | >1000 | >1000 | >1000 | N/A | N/A |
| 6-deoxyerythronolide | S31 | >1000 | >1000 | >1000 | N/A | N/A |
| B | | | | | | |
| Erythromycin B | S32 | >1000 | >1000 | >1000 | N/A | N/A |
| Narbomycin | S33 | >1000 | >1000 | >1000 | N/A | N/A |
| Borrelidin | S2 | 85 | 165 | 91 | 2.0 | 1.5 |
| Rifampicin | S26 | >1000 | >1000 | >1000 | N/A | N/A |
| Tirandamycin C | S8 | >1000 | >1000 | >1000 | N/A | N/A |
| Diphyllin | S1 | >1000 | >1000 | >1000 | N/A | N/A |
| Bafilomycin C | S60 | 1.6 | 4.1 | 3.2 | 2.0 | 2.6 |
| Bafilomycin B | S61 | 312 | 45 | 40 | 3.5 | 3.9 |
| 21-Formyl Baf | A1 | 3.3 | 12 | 7.3 | 2.6 | 3.6 |
| | S61 | | | | | |
| 21-Penta-3-enoate Baf | S29 | 24 | 47 | 39 | 1.6 | 2.0 |
| A1 | | | | | | |
| Bafilomycin A₂ | S4 | 12 | 24 | 13 | 1.2 | 2.3 |
| 19-Deoxy Bafilomycin | S5 | 34 | 130 | 46 | 1.4 | 4.1 |
| A₁ | | | | | | |
| 19-Deoxy-20-ene | S7 | 140 | 360 | 230 | 1.6 | 2.6 |
| Bafilomycin A₁ | | | | | | |
| 19-Deoxy-21-Methoxy | S6 | 20 | 52 | 28 | 1.4 | 2.7 |
| B Bafilomycin A₁ | | | | | | |
| 19-Deoxy-21-allylester | S27 | 59 | 140 | 80 | 1.5 | 2.5 |
| Bafilomycin A₁ | | | | | | |
| 19-Deoxy-21(4- | S28 | 21 | 61 | 36 | 1.7 | 2.8 |
| Pentynoate) | | | | | | |
| Bafilomycin A₁ | | | | | | |
| 7,21-diacetyl | S19 | 250 | 530 | 440 | 1.8 | 2.2 |
| Bafilomycin A₁ | | | | | | |
| 21-Deoxy CMC | S62 | 1.5 | 6.0 | 2.0 | 1.8 | 4.2 |
| 21-Deoxy CMF | S24 | 4.5 | 32 | 9.4 | 2.2 | 8.7 |
| 23-OH-tylactone | S46 | >1000 | >1000 | >1000 | N/A | N/A |
| Des-tylactone | S47 | >1000 | >1000 | >1000 | N/A | N/A |
| 3′,4-diacetyl | S48 | 0.72 | 3.1 | 1.3 | 1.8 | 4.2 |
| CMC | | (0.11) | (1.3) | (0.2) | (0.2) | (1.5) |
| | | 3 | 3 | 3 | 3 | 3 |
| 3′,4′,9-triaceyl | S49 | 2.8 | 11 | 4.5 | 1.8 | 3.6 |
| CMC | | (0.9) | (2.3) | (1.3) | (0.9) | (0.6) |
| | | 3 | 2 | 3 | 3 | 2 |
| 23-Acetyl CMF | S50 | 1.2 | 6.1 | 6.1 | 5.8 | 5.6 |
| | | (0.6) | (0.8) | (0.6) | (2.4) | (1.7) |
| | | 3 | 3 | 3 | 3 | 3 |

### REFERENCES

Bender, T., Huss, M., Wieczorek, H., Grond, S., and von Zezschwitz, P. (2007). Convenient Synthesis of a [1-14C]Diazirinylbenzoic Acid as a Photoaffinity Label for Binding Studies of V-ATPase Inhibitors. European Journal of Organic Chemistry 2007, 3870-3878. https://doi.org/10.1002/ejoc.200700194https://doi.org/10.1002/ejoc.200700194.
Bindseil, K. U., and Zeeck, A. (1993). Chemistry of unusual macrolides. 1. Preparation of the aglycons of concanamycin A and elaiophylin. The Journal of Organic Chemistry 58, 5487-5492. 10.1021/jo00072a036.
Bowman, E. J., Siebers, A., and Altendorf, K. (1988). Bafilomycins: a class of inhibitors of membrane ATPases from microorganisms, animal cells, and plant cells. Proc Natl Acad Sci USA 85, 7972-7976. 10.1073/pnas.85.21.7972.
Collins, K. L., Chen, B. K., Kalams, S. A., Walker, B. D., and Baltimore, D. (1998). HIV-1 Nef protein protects infected primary cells against killing by cytotoxic T lymphocytes. Nature 391, 397-401. 10.1038/34929.
Dröse, S., Boddien, C., Gassel, M., Ingenhorst, G., Zeeck, A., and Altendorf, K. (2001). Semisynthetic Derivatives of Concanamycin A and C, as Inhibitors of V- and P-Type ATPases: Structure-Activity Investigations and Developments of Photoaffinity Probes. Biochemistry 40, 2816-2825. 10.1021/bi001759q.
Gatti, P. A., Gagliardi, S., Cerri, A., Visconti, M., and Farina, C. (1996). Selective Oxidation of Hydroxy Groups of Bafilomycin A1. The Journal of Organic Chemistry 61, 7185-7188. 10.1021/jo9607222.
Ingenhorst, G., Bindseil, Kai U., Boddien, C., Dröse, S., Gaßel, M., Altendorf, K., and Zeeck, A. (2001). Synthesis of a Doubly Labelled Concanamycin Derivative for ATPase Binding Studies. European Journal of Organic Chemistry 2001, 4525-4532. https://doi.org/10.1002/1099-0690(200112)2001:23https://doi.org/10.1002/1099-0690(200112)2001:23<4525::AID-EJOC4525>3.0.CO;2-S.
Keon, K. A., Benlekbir, S., Kirsch, S. H., Müller, R., and Rubinstein, J. L. (2022). Cryo-EM of the Yeast VO Complex Reveals Distinct Binding Sites for Macrolide V-ATPase Inhibitors. ACS Chemical Biology 17, 619-628. 10.1021/acschembio.1c00894.
Kinashi, H., Someno, K., and Sakaguchi, K. (1984). Isolation and characterization of concanamycins A, B and C. J Antibiot (Tokyo) 37, 1333-1343. 10.7164/antibiotics.37.1333.
Li, Z., Du, L., Zhang, W., Zhang, X., Jiang, Y., Liu, K., Men, P., Xu, H., Fortman, J. L., Sherman, D. H., et al. (2017). Complete elucidation of the late steps of bafilomycin biosynthesis in <em>Streptomyces lohii</em>. Journal of Biological Chemistry 292, 7095-7104. 10.1074/jbc.M116.751255.
Osteresch, C., Bender, T., Grond, S., Von Zezschwitz, P., Kunze, B., Jansen, R., Huss, M., and Wieczorek, H. (2012). The Binding Site of the V-ATPase Inhibitor Apicularen Is in the Vicinity of Those for Bafilomycin and Archazolid. Journal of Biological Chemistry 287, 31866-31876. 10.1074/jbc.m112.372169.
Painter, M. M., Zimmerman, G. E., Merlino, M. S., Robertson, A. W., Terry, V. H., Ren, X., McLeod, M. R., Gomez-Rodriguez, L., Garcia, K. A., Leonard, J. A., et al. (2020). Concanamycin A counteracts HIV-1 Nef to enhance immune clearance of infected primary cells by cytotoxic T lymphocytes. Proceedings of the National Academy of Sciences 117, 23835-23846. 10.1073/pnas.2008615117.
Robert-Guroff, M., Popovic, M., Gartner, S., Markham, P., Gallo, R. C., and Reitz, M. S. (1990). Structure and expression of tat-, rev-, and nef-specific transcripts of human immunodeficiency virus type 1 in infected lymphocytes and macrophages. J Virol 64, 3391-3398. 10.1128/jvi.64.7.3391-3398.1990.
Roeth, J. F., Williams, M., Kasper, M. R., Filzen, T. M., and Collins, K. L. (2004). HIV-1 Nef disrupts MHC-I trafficking by recruiting AP-1 to the MHC-I cytoplasmic tail. Journal of Cell Biology 167, 903-913. 10.1083/jcb.200407031.
Siliciano, J. D., Kajdas, J., Finzi, D., Quinn, T. C., Chadwick, K., Margolick, J. B., Kovacs, C., Gange, S. J., and Siliciano, R. F. (2003). Long-term follow-up studies confirm the stability of the latent reservoir for HIV-1 in resting CD4+ T cells. Nature Medicine 9, 727-728. 10.1038/nm880.
Sørensen, M. G., Henriksen, K., Neutzsky-Wulff, A. V., Dziegiel, M. H., and Karsdal, M. A. (2007). Diphyllin, a novel and naturally potent V-ATPase inhibitor, abrogates acidification of the osteoclastic resorption lacunae and bone resorption. J Bone Miner Res 22, 1640-1648. 10.1359/jbmr.070613.
Wang, R., Wang, J., Hassan, A., Lee, C.-H., Xie, X.-S., and Li, X. (2021). Molecular basis of V-ATPase inhibition by bafilomycin A1. Nature Communications 12. 10.1038/s41467-021-22111-5.
Yoshimori, T., Yamamoto, A., Moriyama, Y., Futai, M., and Tashiro, Y. (1991). Bafilomycin A1, a specific inhibitor of vacuolar-type H(+)-ATPase, inhibits acidification and protein degradation in lysosomes of cultured cells. J Biol Chem 266, 17707-17712.

What is claimed is:

1. A compound having a structure of Formula (I), or a pharmaceutically acceptable salt thereof: 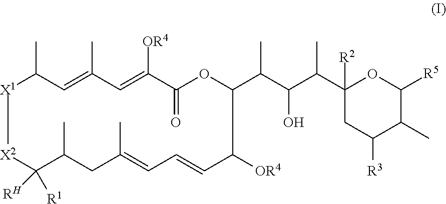
wherein 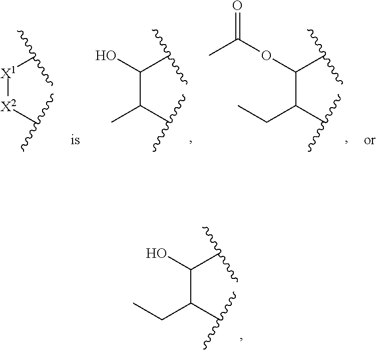
or represents a direct bond;
R^{H} is H;
R¹ is OH, C(O)C₁₋₈ alkyl, or OC(O)C₁₋₈ alkyl,
or R^{H} and R¹ together with the carbon to which they are attached form an oxo (═O) group;
R² is H, OH, or OCH₃;
R³ is CHO, C(O)C₁₋₈ alkyl, OC(O)R⁶, or a modified sugar;
each R⁴ is independently H or C₁₋₆alkyl;
R⁵ is C₁₋₆alkyl or C₂₋₆alkenyl; and
R⁶ is H, C₁₋₆haloalkyl, or C₂₋₆ alkynyl.

2. The compound or salt of claim 1, wherein 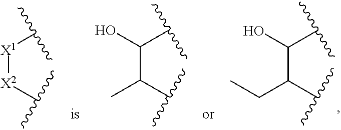
or represents a direct bond.

3. The compound or salt of claim 1 or 2, having a structure of Formula (Ia), (Ib), or (Ic): 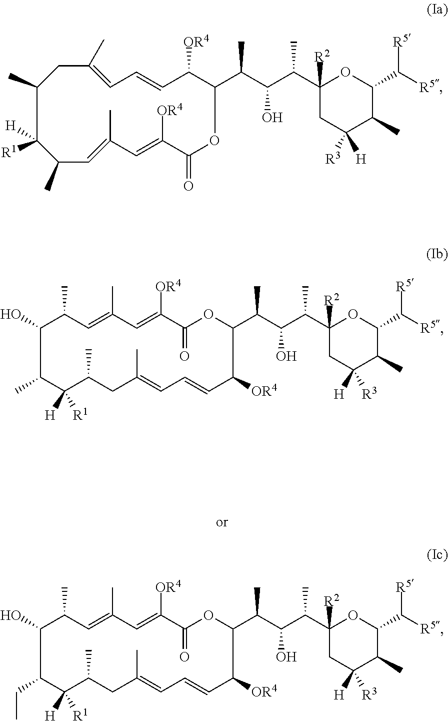
wherein (i) R^{5′} is C₂₋₅alkenyl and R^{5″} is H, or (ii) both R^{5′} and R^{5″} are C₁₋₂alkyl.

4. The compound or salt of any one of claims 1 to 3, wherein R¹ is OH or OC(O)C₁₋₈ alkyl.

5. The compound or salt of any one of claims 1 to 3, wherein R¹ is OH.

6. The compound or salt of any one of claims 1 to 3, wherein R¹ is OC(O)C₁₋₃ alkyl.

7. The compound or salt of claim 6, wherein R¹ is

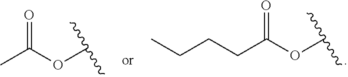

8. The compound or salt of claim 1, wherein R^{H} and R¹ together with the carbon to which they are attached form an oxo (═O) group.

9. The compound or salt of any one of claims 1 to 8, wherein R² is H.

10. The compound or salt of any one of claims 1 to 8, wherein R² is OH.

11. The compound or salt of any one of claims 1 to 8, wherein R² is OCH₃.

12. The compound or salt of any one of claims 1 to 11, wherein R³ is OC(O)R⁶ or a modified sugar.

13. The compound or salt of any one of claims 1 to 12, wherein R³ is OC(O)R⁶.

14. The compound or salt of claim 13, wherein R⁶ is H.

15. The compound or salt of claim 13, wherein R⁶ is C₁₋₆haloalkyl.

16. The compound or salt of claim 15, wherein R³ is 2-fluoroacetyl, 2-chloroacetyl, 2-bromoacetyl, or 2-bromopropanoate.

17. The compound or salt of claim 13, wherein R⁶ is C₂₋₆ alkynyl.

18. The compound or salt of claim 17, wherein R³ is

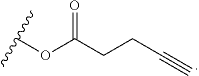 [SMILES: *OC(=O)CCC#C]

19. The compound or salt of any one of claims 1 to 12, wherein R³ is 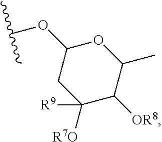
wherein
R⁷ is H, C(O)C₁₋₈alkyl, C(O)C₂₋₈alkenyl, or C(O)C₂₋₈alkynyl,
or OR⁷ and R⁹ together with the carbon to which they are attached form an oxo (═O) group;
R⁸ is H, C(O)C₁₋₈alkyl, C(O)C₂₋₈alkenyl, C(O)C₂₋₈alkynyl, or C(O)NH₂; and
R⁹ is H;
wherein C₂₋₈alkynyl is optionally substituted with a fused 3-membered heterocycloalkyl ring comprising two nitrogen atoms.

20. The compound or salt of claim 19, wherein R³ is

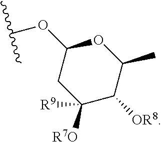

21. The compound or salt of claim 20, wherein R³ is

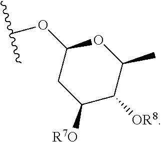

22. The compound or salt of any one of claims 19 to 21, wherein R⁷ is H.

23. The compound or salt of any one of claims 19 to 21, wherein R⁷ is C(O)C₁₋₈alkyl, C(O)C₂₋₈alkenyl, or C(O)C₂₋₈alkynyl.

24. The compound or salt of claim 23, wherein R⁷ is

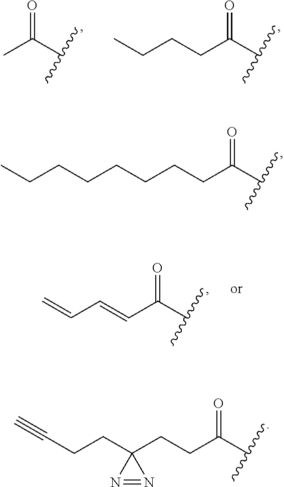

25. The compound or salt of claim 19 or 20, wherein OR⁷ and R⁹ together with the carbon to which they are attached form an oxo (═O) group.

26. The compound or salt of any one of claims 19 to 25, wherein R⁸ is H.

27. The compound or salt of any one of claims 19 to 25, wherein R⁸ is C(O)C₁₋₈alkyl.

28. The compound or salt of claim 27, wherein R⁸ is

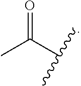 [SMILES: *C(C)=O]

29. The compound or salt of any one of claims 19 to 25, wherein R⁸ is C(O)NH₂.

30. The compound or salt of any one of claims 1 to 29, wherein at least one R⁴ is C₁₋₆alkyl.

31. The compound or salt of any one of claims 1 to 30, wherein each R⁴ is C₁₋₆alkyl.

32. The compound or salt for use of claim 30 or 31, wherein at least one R⁴ is methyl.

33. The compound or salt of claim 32, wherein each R⁴ is methyl.

34. The compound or salt of any one of claims 1 to 33, wherein R⁵ is C₁₋₆alkyl.

35. The compound or salt of any one of claims 1 to 33, wherein R⁵ is C₂₋₆alkenyl.

36. The compound or salt of any one of claims 3 to 35, wherein R^{5′} is C₂₋₅alkenyl and R^{5″} is H.

37. The compound or salt of claim 36, wherein R⁵ is

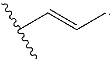 [SMILES: */C=C/C]

38. The compound or salt of any one of claims 3 to 35, wherein both R^{5′} and R^{5′} are C₁₋₂alkyl.

39. The compound or salt of claim 38, wherein R⁵ is

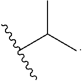 [SMILES: *C(C)C]

40. A compound, or pharmaceutically acceptable salt thereof, having a structure as shown in Table A.

41. The compound or salt any one of claims 1 to 40 in the form of a salt.

42. A pharmaceutical composition comprising the compound of salt of any one of claims 1 to 40 and a pharmaceutically acceptable excipient.

43. A method of modulating human immunodeficiency virus (HIV) Nef and its allotypes comprising administering to a patient in need thereof a pharmaceutically-effective amount of the compound or salt of any one of claims 1 to 41 or the pharmaceutical composition of claim 42.

44. The method of claim 43, wherein modulating HIV Nef and its allotypes comprises inhibiting HIV Nef and its allotypes.

45. A method of treating human immunodeficiency virus (HIV) infection comprising administering to a patient in need thereof a pharmaceutically-effective amount of the compound or salt of any one of claims 1 to 41 or the pharmaceutical composition of claim 42.

46. The method of claim 45, wherein the HIV infection is HIV-1 infection.

47. The method of claim 46, wherein the HIV-1 infection is infection with HIV subtype A, B, C, D, E, F, G, H, I, J, K, L, or a recombination thereof.

48. The method of any one of claims 45 to 47, wherein treating HIV infection comprises reducing an HIV reservoir in a host.

49. The method of any one of claims 45 to 48, wherein treating HIV infection comprises eliminating an HIV reservoir in a host.

50. The compound or salt of any one of claims 1 to 41 or the composition of claim 42 for use as a medicament for modulating human immunodeficiency virus (HIV) Nef and its allotypes in a patient.

51. The compound or composition of claim 50, wherein modulating HIV Nef and its allotypes comprises inhibiting HIV Nef and its allotypes.

52. The compound or salt of any one of claims 1 to 41 or the composition of claim 42 for use as a medicament for treating human immunodeficiency virus (HIV) infection in a patient.

53. The compound or composition of claim 52, wherein the HIV infection is HIV-1 infection.

54. The compound or composition of claim 53, wherein the HIV-1 infection is infection with HIV subtype A, B, C, D, E, F, G, H, I, J, K, L, or a recombination thereof.

55. The compound or composition of any one of claims 52 to 54, wherein treating HIV infection comprises reducing an HIV reservoir in a host.

56. The compound or composition of any one of claims 52 to 55, wherein treating HIV infection comprises eliminating an HIV reservoir in a host.
